(19) United States
(12) Patent Application Publication
(10) Pub. No.: US 2026/0306543 A1
(43) Pub. Date: Oct. 8, 2026

(54) INTRA-STRIATAL CO-TRANSPLANTATION OF AUTOLOGOUS TREG AND MDA CELLS IN PARKINSON'S DISEASE CELL THERAPY

(71) Applicant: THE MCLEAN HOSPITAL CORPORATION, Belmont, MA (US)

(72) Inventors: Kwang S. Kim, Woburn, MA (US); Tae-Yoon Park, Cambridge, MA (US)

(21) Appl. No.: 19/482,362

(22) PCT Filed: May 9, 2024
(86) PCT No.: PCT/US2024/028596

§ 371 Date: Nov. 7, 2025

Related U.S. Application Data
(60) Provisional application No. 63/500,941, filed on May 9, 2023.

(51) Int. Cl. A61K 35/17; A61K 9/00; A61K 35/30; A61K 38/00; A61K 40/11; A61K 40/50; A61P 25/16; C07K 16/22; C07K 16/24; C12N 5/079

(52) U.S. Cl. CPC A61K 35/17; A61K 9/0085; A61K 35/30; A61K 38/00; A61K 40/11; A61K 40/50; A61P 25/16; C07K 16/22; C07K 16/241; C07K 16/245; C07K 16/249; C12N 5/0618; C12N 2506/45

(57) ABSTRACT

Described herein, inter alia, are compositions and methods of use (e.g., treating Parkinson's Disease and/or reducing the immune response due to needle trauma during cell transplantation) for administering a population of regulatory T (T_{REG}) cells and/or a population of midbrain dopamine (mDA) cells to the brain of a subject.

### CLAIM OF PRIORITY

This application claims the benefit of U.S. Provisional Application Ser. No. 63/500,941, filed on May 9, 2023. The entire contents of the foregoing are incorporated herein by reference.

### BACKGROUND

The cardinal motor symptoms of Parkinson's disease (PD), including tremor, rigidity, bradykinesia and postural instability, are caused by selective and progressive degeneration of midbrain dopamine (mDA) neurons (mDANs) in the substantia nigra, rendering cell replacement therapy a promising therapeutic strategy, a concept supported by the results of previous fetal ventral mesencephalic (VM) cell transplantation studies¹⁻⁶. The clinical use of human fetal VM cells is limited by ethical and logistical considerations. mDA cells derived from human pluripotent stem cells such as human induced pluripotent stem cells (hiPSC) or embryonic stem cells (hESC) are emerging as potential alternative cell sources¹⁻⁶. Regardless of the source of transplantable cells, a fundamental requirement of cell replacement therapy is survival of grafted cells through the transplantation process Therefore, numerous investigators studied the survival of transplanted cells as a crucial requirement for successful PD cell therapy, mostly using human and rodent embryonic VM cells⁷⁻¹¹ These early studies revealed that a great majority (80-99%) of transplanted mDANs undergo apoptotic cell death within 1-2 weeks post-transplantation. Notably, these studies also revealed that the number of surviving mDA neurons subsequently remained the same at later time points (e.g., at 4 days, 2 and 6 weeks post-transplantation), further supporting the view that most transplanted mDA neurons suffered an early death and that no new mDA neurons were generated from transplanted VM cells. Many potential mechanisms were proposed to explain the poor survival and early death of grafted mDA neurons, mostly focusing on interactions between grafted cells and the host brain such as lack of proper supply with oxygen, glucose, or growth factors from the host brain in the immediate vicinity of the fresh graft. Accordingly, numerous efforts have been made to improve graft survival using diverse protective factors including calcium channel antagonists, lazaroids, caspase inhibitors, and trophic factors⁴⁰⁻⁴⁴.

The use of autologously derived cells for transplantation may reduce the host immune response. Autologous mDA cell transplantation has recently been reported in nonhuman primate PD models¹²⁻¹⁴. In another investigation, neural grafting of major histocompatibility complex (MHC)-matched primate iPSC reduced the host immune response and increased the survival of grafted mDA neurons¹⁵. Moreover, autologous hiPSC-derived mDA cells were successfully transplanted into the striatum of a sporadic PD patient without the use of immunosuppressants, and also showed that mice with humanized immune systems constructed from the peripheral blood mononuclear cells (PBMC) of that patient did not reject such grafts¹⁶. Although these data showed that using autologous mDA cells can avoid graft rejection, cell survival and functional recovery in the first patient as assessed by ¹⁸F-DOPA PET scan analysis and clinical motor assessments was modest¹⁶, suggesting that survival of mDA neurons may have been limited even with autologous transplantation. To compensate for this poor survival, some investigators have proposed transplanting enormous numbers of cells, such as a recent clinical trial in Australia, which proposed to transplant as many as 70 million cells (neuroscience.org.au/research-news/parkinsons-stem-cell-trial). In addition, some investigators have suggested using earlier differentiation stage cells (e.g., Day 16-17) for transplantation^{17,18}. Although these approaches are interesting, the risk of undesirable side effects including overgrowth of grafts, disruption of host brain structure, and/or tumor formation need to be carefully examined.

### SUMMARY

This application is based, at least in part, on the surprising discovery that co-administering a population of regulatory T (T_{REG}) cells with a population of midbrain dopamine cells (mDA cells) into the brain of a subject (e.g., a subject suffering from Parkinson's disease) led to better outcomes for the subject (e.g., decreased immune response in the subject). Useful mDA cells can comprise midbrain dopamine neurons (mDANs), midbrain dopamine progenitor cells (mDAPs), and/or combinations thereof. This application is also based, at least in part, on the surprising discovery that administering a population of T_{REG} cells with a population of desired cells (e.g., neurons) in a cell therapy reduced needle trauma (e.g., reduced the host's immune response), reduced graft overgrowth, and increased cell survival of the desired cell population.

The role of the immediate host brain tissue reaction via the innate immune response to the surgical injury of transplantation, and how this affects, in particular, the survival of the desired tyrosine hydroxylase (TH)⁺ mDA neurons within the graft, has not been specifically investigated. Herein it is shown that a great majority (~90%) of hiPSC-derived mDA neurons present within the initial population of grafted cells died within 1-2 weeks post-transplantation while the remaining TH cells in the graft mostly survived whether they are transplanted into either immunodeficient or immunocompetent host animals, including humanized mice. We showed that an important factor in this early death of mDA neurons is the surgical trauma itself (herein referred to as “needle trauma”), which triggers an acute host neuroinflammatory response that has a preferential adverse effect on TH⁺ mDA neurons compared to TH cells within the graft. In addition, we found that this needle trauma damages the host brain and significantly kills host neuronal cells (FIG. 7g).

Regulatory T cells (T_{REG}) are indispensable to the maintenance of normal immunological tolerance and homeostasis and are so named due to their modulation of other immune cells to contain inflammatory and immune rejection responses^{19,20} Thus, co-transplantation of autologous T_{REG} during surgical implantation of the graft might effectively and safely help suppress the host inflammatory response to the needle trauma. We found that intra-striatal co-transplantation of autologous T_{REG} significantly protects both host neurons and grafted mDA neurons from this needle trauma-induced death, further improves behavioral recovery in 6-OHDA lesioned PD rodent models and reduces proliferation of TH⁻ cells within the graft. Because T_{REG} are known to suppress inflammatory response, they are being used to control inflammation in clinical settings. However, T_{REG} are administered to patients systemically through intravenous injection and their effects on the survival of mDA neurons following intra-striatal injection are unknown. Thus, we tested the effects of conventional T_{REG} therapy using adoptive transfer^{27,28} by intravenously injecting about 1,000,000 ex vivo expanded autologous CD4⁺CD25⁺ T_{REG} (Foxp3⁺, 68.8±4.07%) (FIG. 9b) and compared it to direct intra-striatal transplantation. Although adoptive transfer reduced infiltration of MHCII⁺ cell by approximately 50%, it was significantly less efficient than direct intra-striatal transplantation of 20,000 T_{REG} (FIG. 1n). In short, although only 2% of cells was used, direct intra-striatal injection of T_{REG} is much more effective than the conventional adoptive transfer.

Accordingly, described herein, inter alia, are methods of treating Parkinson's Disease (PD) in a patient by co-transplanting T_{REG} cells and mDA neurons into the brain of the patient. The T_{REG} cells and mDA neurons can be transplanted simultaneous or one directly after the other. The T_{REG} cells and mDA neurons can be autologous T_{REG} cells and autologous mDA neurons. Alternatively, the T_{REG} cells and mDA neurons can be allogeneic, or xenogeneic. The T_{REG} cells and mDA neurons can be transplanted intra-striatally. The T_{REG} cells and mDA cells can be transplanted in the amounts of between 5,000 and 100,000 cells and between 2 million and 20 million mDA cells, respectively.

In various embodiments of methods of treating PD: the T_{REG} cells and the mDA cells are transplanted simultaneously; the T_{REG} cells and the mDA cells are transplanted one directly after the other; the T_{REG} cells are autologous T_{REG} cells, allogeneic T_{REG} cells, or xenogeneic T_{REG} cells; the mDA cells are autologous mDA cells, allogeneic mDA cells, or xenogeneic mDA cells; the T_{REG} cells and mDA cells are transplanted intra-striatally.

Also described herein are methods of decreasing and/or suppressing a host immune response triggered by needle trauma in a patient undergoing cell transplantation comprising co-transplantating T_{REG} cells with the cells being transplanted. In some embodiments, the cells being transplanted can be mDA cells (e.g., mDA neurons, mDA progenitors, and mixtures thereof). In some embodiments, the T_{REG} cells and mDA cells can be transplanted simultaneous or one directly after the other. In some embodiments, the T_{REG} cells and mDA cells can be autologous T_{REG} cells (that is, from the patient) and autologous mDA cells. In some embodiments, the, the patient-derived T_{REG} cells can be used with mDA cells that can be allogeneic, or xenogeneic. In some embodiments, the T_{REG} cells and mDA cells can be transplanted intra-striatally. In some embodiments, the T_{REG} cells and mDA cells can be transplanted in the amounts between 5,000 and 100,000 cells and between 2 million and 20 million mDA cells respectively.

In various embodiments: the T_{REG} cells and the mDA cells are transplanted simultaneously; the T_{REG} cells and the mDA cells are transplanted one directly after the other; the T_{REG} cells are autologous T_{REG} cells, allogeneic T_{REG} cells, or xenogeneic T_{REG} cells; the mDA cells are autologous mDA cells, allogeneic mDA cells, or xenogeneic mDA cells; the T_{REG} cells and mDA cells are transplanted intra-striatally.

In addition, an unexpected finding is that co-transplantation of T_{REG} significantly rescues the death of host brain cells that are caused by needle trauma compared to control groups (FIGS. 7e,7f,7g; FIGS. 1h-1k), demonstrating that co-transplantation of T_{REG} will protect patient's (host) neurons from death.

Also described herein, inter alia, are methods of improving outgrowths from grafts of mDA cells upon transplantation into a patient comprising co-transplanting T_{REG} cells with the transplanted mDA cells, wherein the co-transplantation results in a high proportionate TH+ cell content in the graft in contrast to a graft of mDA cells alone. In some embodiments, the T_{REG} cells and mDA neurons can be transplanted simultaneous or one directly after the other. In some embodiments, the T_{REG} cells and mDA neurons can be autologous T_{REG} cells and autologous mDA neurons. In some embodiments, the, the T_{REG} cells and mDA cells can be allogeneic or xenogeneic. In some embodiments, the T_{REG} cells and mDA neurons can be transplanted intra-striatally. In some embodiments, the T_{REG} cells and mDA neurons can be transplanted in the amounts of between 5,000 and 100,000 cells and between 2 million and 20 million mDA cells respectively.

In various embodiments: the T_{REG} cells and the mDA cells are transplanted simultaneously; the T_{REG} cells and the mDA cells are transplanted one directly after the other; the T_{REG} cells are autologous T_{REG} cells, allogeneic T_{REG} cells, or xenogeneic T_{REG} cells; the mDA cells are autologous mDA cells, allogeneic mDA cells, or xenogeneic mDA cells; the T_{REG} cells and mDA cells are transplanted intra-striatally.

In addition, abnormal outgrowth of TH-cells may disrupt the brain structure of the patient and may lead to adverse effects and/or side effects. In this regard, the co-transplantation of the T_{REG} cells and mDA cells has the advantage by reducing the overall size of the graft and the number of Ki67+ proliferating cells and enhance the safety of cell therapy.

Furthermore, we found that needle trauma preferentially kills not only mDA neurons but also other types of neurons such as GABAergic neurons, compared to non-neuronal proliferating cells. Thus, co-transplantation of T_{REG} can benefit the survival of not only mDA neurons but also other types of neurons and can be applied to benefit both efficacy and safety of cell replacement therapy for Parkinson's disease and other neurodegenerative disease.

Last but not least, we found that co-transplantation of specific antibodies against pro-inflammatory cytokines (such as FDA approved: Infliximab (anti-TNF-α), Canakinumab (anti-IL-1β), Emapalumab (anti-IFN-γ)) or co-transplantation with an antibody against a damage-associated molecular patterns (DAMPs) molecule (such as, an anti-HMGB1 Ab, an anti-IL-1α Ab) also exhibit protective effects against needle trauma-induced death of host brain cells and grafted mDA neurons. Moreover, we found that administration of anti-inflammatory drugs (such as cyclosporin A, dexamethozone, and FK506) show protective effects. Thus, these methods/reagents can be also used instead of T_{REG} either alone or in combinations.

In some embodiments, described herein are methods of treating a subject having Parkinson's Disease (PD), the methods comprising administering a population of regulatory T (T_{REG}) cells and a population of midbrain dopamine (mDA) cells to the brain of the subject. In some embodiments, described herein are methods of treating a subject having Parkinson's Disease (PD), the methods comprising administering to the subject a population of midbrain dopamine (mDA) cells and an antibody (Ab) (optionally, an antibody that binds a pro-inflammatory cytokine or a damage-associated molecular patterns (DAMPs) molecule; optionally an anti-TNF-α Ab (optionally Infliximab), an anti-IL-1β Ab (optionally Canakinumab), an anti-IFN-γ Ab (optionally Emapalumab), an anti-HMGB1 Ab, an anti-IL-1α Ab).

In some embodiments, described herein are methods of decreasing an immune response in a subject (e.g., an immune response triggered by needle trauma) during cell transplantation, the methods comprising administering to the subject an effective amount of a population of T_{REG} cells with a second population of cells (“transplanted cells”; optionally mDA cells). In some embodiments, described herein are methods of decreasing an immune response in a subject (e.g., an immune response triggered by needle trauma) during a cell transplantation comprising administering to the subject an effective amount of an antibody (Ab) (optionally an antibody that binds a pro-inflammatory cytokine or a damage-associated molecular patterns (DAMPs) molecule; optionally an anti-TNF-α Ab (optionally Infliximab), an anti-IL-1β Ab (optionally Canakinumab), an anti-IFN-γ Ab (optionally Emapalumab), an anti-HMGB1 Ab, or an anti-IL-1α Ab) with a population of cells (optionally mDA cells).

In some embodiments, described herein are methods of increasing cell survival of transplanted cells (optionally mDANs) in cell transplantation and/or reducing overgrowth of cells (optionally of TH-cells, Ki67+ cells, and/or cells other than mDANs) during a cell transplantation, the methods comprising co-transplanting an effective amount of a population of T_{REG} cells with a second population of cells (“transplanted cells”; optionally mDA cells). In some embodiments, described herein are methods of increasing cell survival of transplanted cells (optionally, mDANs) in cell transplantation and/or reducing overgrowth of cells (optionally, TH− cells, Ki67⁺ cells, and/or cells other than mDANs) during a cell transplantation, the methods comprising administering to the subject an effective amount of an antibody (Ab) (optionally, an antibody that binds a pro-inflammatory cytokine or a damage-associated molecular patterns (DAMPs) molecule; optionally an anti-TNF-α Ab (optionally Infliximab), an anti-IL-1β Ab (optionally Canakinumab), an anti-IFN-γ Ab (optionally Emapalumab), an anti-HMGB1 Ab, or an anti-IL-1α Ab) with a second population of cells (optionally mDA cells).

In some embodiments of any of the methods described herein, the transplanted cells are mDA cells. In some embodiments of any of the methods described herein, the second population of cells is a population of mDA cells. In some embodiments of any of the methods described herein, the population of mDA cells comprises midbrain dopamine neurons (mDANs) and midbrain dopamine progenitor cells (mDAPs). In some embodiments of any of the methods described herein, a population of mDA cells comprises at least about 50, 55, 60, 65, 70, 75, 80, 85, 90, or 95% mDAPs and at least about 5, 10, 15, 20, or 25% mDANs.

In some embodiments of any of the methods described herein, the ratio of mDA cells: Treg cells is at least about 1:1, 1:2, 1:3, 1:4, or 1:5. In some embodiments of any of the methods described herein, the T_{REG} cells and the mDA cells are administered to the subject at the same time, optionally mixed together in a single composition. In some embodiments of any of the methods described herein, the T_{REG} cells are first administered to the subject and subsequently the mDA cells are administered to the subject. In some embodiments of any of the methods described herein, the mDA cells are first administered to the subject and subsequently the T_{REG} cells are administered to the subject. In some embodiments of any of the methods described herein, the T_{REG} cells and the mDA cells are transplanted as a single composition in a single dose. In some embodiments of any of the methods described herein, the T_{REG} cells and the mDA cells are transplanted as a single composition in repeat doses.

In some embodiments of any of the methods described herein, the T_{REG} cells and the mDA cells are autologous T_{REG} cells and autologous mDA cells. In some embodiments of any of the methods described herein, the T_{REG} cells and the mDA cells are xenogeneic T_{REG} cells and xenogeneic mDA cells. In some embodiments of any of the methods described herein, the T_{REG} cells are autologous T_{REG} cells, allogenic T_{REG} cells, or xenogeneic T_{REG} cells. In some embodiments of any of the methods described herein, the mDA cells are autologous mDA cells, allogenic mDA cells, or xenogeneic mDA cells. In some embodiments of any of the methods described herein, the transplanted cells (optionally mDA cells) are autologous transplanted cells, allogenic transplanted cells (optionally mDA cells), or xenogeneic transplanted cells (optionally mDA cells).

In some embodiments of any of the methods described herein, the autologous mDA are obtained by in vitro differentiation of human induced pluripotent stem cells (hiPSCs) into mDA cells, wherein the hiPSC are derived from cells from the subject. In some embodiments of any of the methods described herein, the T_{REG} cells and the mDA cells are allogeneic or T_{REG} cells and allogeneic mDA cells, optionally wherein the autologous mDA are obtained by in vitro differentiation of human pluripotent stem cells (hPSCs), optionally human embryonic stem cells (hESCs) and induced pluripotent stem cells (hiPSCs), into mDA cells.

In some embodiments of any of the methods described herein, the T_{REG} cells and mDA cells are administered intrastriatally, optionally by intra-striatal injection.

In some embodiments of any of the methods described herein, the T_{REG} cells are transplanted intrastriatally. In some embodiments of any of the methods described herein, the mDA cells are transplanted intrastriatally. In some embodiments of any of the methods described herein, the transplanted cells (optionally mDA cells) are transplanted intrastriatally.

In some embodiments of any of the methods described herein, the co-transplanting results in a graft, and wherein there is an increase in TH⁺ cells in the graft compared to a graft without the co-transplanting. In some embodiments of any of the methods described herein, the co-transplanting results in a graft, and wherein there is an increase in the TH⁺ cells: TH⁻ cells ratio in the graft and/or an increase in the TH⁺ cells: Ki67⁺ cells ratio in the graft compared to a graft without the co-transplanting. In some embodiments of any of the methods described herein, the co-transplanting results in a graft, and wherein there is a decrease in Ki67⁺ cells in the graft and/or a decrease in TH cells in the graft compared to a graft without the co-transplanting.

In some embodiments of any of the methods described herein, the T_{REG} cells and the transplanted cells (optionally mDA cells) are transplanted simultaneously. In some embodiments of any of the methods described herein, the T_{REG} cells and the transplanted cells (optionally mDA cells) are transplanted separately. In some embodiments of any of the methods described herein, the T_{REG} cells are first administered to the subject and subsequently the transplanted cells (optionally mDA cells) are administered to the subject. In some embodiments of any of the methods described herein, the transplanted cells (optionally mDA cells) are first administered to the subject and subsequently the T_{REG} cells administered to the subject. In some embodiments of any of the methods described herein, the ratio of transplanted cells: Treg cells is at least about 1:1, 1:2, 1:3, 1:4, or 1:5. In some embodiments of any of the methods described herein, the T_{REG} cells and the transplanted cells are administered to the subject at the same time, optionally mixed together in a single composition. In some embodiments of any of the methods described herein, the T_{REG} cells and the transplanted cells are administered to the subject in a single composition as a single dose. In some embodiments of any of the methods described herein, the T_{REG} cells and the transplanted cells are administered to the subject in a single composition as repeat doses.

In some embodiments of any of the methods described herein, an effective amount of T_{REG} cells and an effective amount mDA cells are administered to the subject. In some embodiments of any of the methods described herein, the amount T_{REG} cells and mDA cells administered to the subject are sufficient to improve one or more symptoms of PD in the subject, thereby treating PD. In some embodiments of any of the methods described herein, the amount T_{REG} cells administered to the subject is sufficient to reduce or eliminate the amount (and/or percentage) of at least one pro-inflammatory cytokine (e.g., TNF-α, IL-1β, and/or IFN-γ) and/or, and/or at least one damage-associated molecular patterns (DAMPs; e.g., HMGB1) at and/or near the injection site.

As used herein, a cell transplantation includes any therapy in which a population of cells is administered (e.g., via injection, infusion, graft/transplantation) to a subject, such as a cell therapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1a-1n. Influence of needle trauma-induced neuroinflammation on transplanted cells. 1a-1g, NSG or C4-hu mice were sacrificed 14 days after intra-striatal transplantation of C4-mDAPs. The total number and percentage of TH⁺ mDA neurons were determined by immunofluorescence staining with anti-TH/Hoechst 33342 before transplantation (1a, 1c, 1d) or immunohistochemistry staining with anti-TH/hNUCLEI antibodies after transplantation (1b, 1c, 1d). (One-way ANOVA, Tukey's post-hoc test; n=4 per group). The level of inflammatory cell infiltration was confirmed by immunofluorescence staining with anti-hIbal (1e), -hCD45 (1f), and hCD4 (1g) antibodies. 1h-1k, Inhibition of needle trauma-induced host inflammatory response using T_{REG}. Fischer 344 rats were sacrificed 7 days after intra-striatal co-transplantation of TP medium with or without autologous T_{REG} from each rat (20 k cells/rat). The level of inflammatory cell infiltration into needle track was determined by immunohistochemical staining with anti-Ibal (1h) and -MHCII (i) antibodies. 1j, The level of needle trauma-induced neuroinflammation was determined by immunofluorescence staining with anti-IFN-γ antibody. 1k, The level of neuroinflammation-induced cell death by needle trauma was analyzed by TUNEL assay. (Student's t-test; n=5 per group). 1l-1n, Advantages of co-transplantation of autologous T_{REG}. The level of inflammatory cell infiltration into needle track was determined by immunohistochemical staining with anti-MHCII antibody. 1l, The inhibitory effect of neuroinflammation by needle trauma was compared 7 days after co-transplantation of syngeneic and autologous T_{REG} (20 k cells/rat). 1m, Inhibition of needle trauma-induced neuroinflammation was measured 7 days after co-transplantation with 2 k, 20 k or 100 k autologous T_{REG} per rat. 1n, Adoptive transfer of ex vivo expanded autologous T_{REG} (1,000 k cells/rat) or intra-striatal transplantation of ex vivo expanded autologous T_{REG} (20 k cells/rat) obtained from each rat was compared after 7 days. (One-way ANOVA, Tukey's post-hoc test; n=5 per group). Each error bar represents means±s.e.m. ns, not significant; **, P<0.01; ***, P<0.001.

FIGS. 2a-2f. Pro-inflammatory cytokine-induced immunogenicity in transplanted C4-mDAPs. a-c, C4-mDAPs were incubated in vitro with 100 ng/ml IFN-γ for 2 days. The level of IFN-γ-induced immunogenicity was determined by flow cytometry analysis with anti-HLA-ABC/HLA-DR (2a), anti-CD80/CD86/CD40/PD-L1/PD-L2/CD47 antibodies (2b), and by western blot analysis with anti-Zg16/Hormad1 antibodies (2c). 2d-2f, MLR-like co-culture assays using C4-mDAPs and C4/K2-PBMC. 2d, The level of T cell activation measured by flow cytometry analysis with anti-CD3/CD69 antibodies. 2e, The level of immunogenicity-induced cell death determined by flow cytometry analysis with anti-hNCAM/Annexin-V staining. 2f, The level of IFN-γ secretion measured by ELISA. †; Activated with Dynabeads™ Human T-Activator. (One-way ANOVA, Tukey's post-hoc test; n=3). Each error bar represents means±s.e.m. **, P<0.01; ***, P<0.001.

FIGS. 3a-3l. Pro-inflammatory cytokine-induced cell death of C4-mDAPs. 3a-3i, C4-mDAPs were incubated in vitro with 100 ng/ml IFN-γ for 7 days. The level of IFN-γ-induced cell death was determined by western blot with anti-cleaved caspase-3 antibody (3a), flow cytometry analysis of Annexin-V/7-AAD staining (3b), and TUNEL assay (3c). (Student's t-test; n=3 biologically independent experiments). TH levels were assayed by immunofluorescence staining (3d), and the number of total cells (3e) or TH⁺ cells (3f), TH⁺ mean fluorescence intensity (3g), and TH⁺ neurite length (3h) were measured. (Student's t-test; n>4). 3i, Apoptotic cell death (Annexin-V⁺) was analyzed by gating TH⁺ and TH⁻ cells using flow cytometry. (One-way ANOVA, Tukey's post-hoc test; n=6). 3j-3l, C4-mDAPs with or without T_{REG} were co-cultured for 7 days under in vitro inflammatory conditions (with or without 100 ng/ml IFN-γ). 3j, Immunofluorescence staining of TH and Ki67. Percentages of TH″ (3k) and Ki67⁺ (3l) cells among total cells. (One-way ANOVA, Tukey's post-hoc test; n=5). Scale bars: 100 μm. Each error bar represents means±s.e.m. ns, not significant; *, P<0.05; **, P<0.01; ***, P<0.001.

FIGS. 4a-4h. Effects of T_{REG} co-transplantation with C4-mDAPs in xenogeneic PD model. 4a-4h, Fischer 344 rats were sacrificed 2 or 20 weeks after intra-striatal co-transplantation of C4-mDAPs with or without autologous T_{REG} (20 k cells/rat) and CsA (control). 4a, 4b, Amphetamine-induced rotation scores were determined every 4 weeks for 20 weeks after transplantation. (Two, One-way ANOVA, Tukey's post-hoc test; n=5 per group). 4c, At 2 weeks, the level of inflammatory cell infiltration was determined by immunohistochemical staining with anti-MHCII antibody. 4d, The level of neuroinflammation-induced cell death was analyzed by TUNEL assay after 2 weeks. 4e, 4g, The total number of C4-mDAPs was confirmed through hNUCLEI staining after 2 weeks (4e) and 20 weeks (4g). 4f, 4h, The total number of TH⁺ cells was determined after 2 weeks (4f) and 20 weeks (4h), respectively, through immunofluorescence staining. (One-way ANOVA, Tukey's post-hoc test; n=5 per group). Each error bar represents means±s.e.m. ns, not significant; *, P<0.05; **, P<0.01; ***, P<0.001.

FIGS. 5a-5m. In vivo effects of T_{REG} CO-transplantation with C4-mDAPs. 5a-5m, NSG mice were sacrificed 2 or 20 weeks after intra-striatal co-transplantation of C4-mDAPs with or without autologous C4-T_{REG} (20 k cells/mouse). 5a, The amphetamine-induced rotation test was performed every 4 weeks after transplantation for 24 weeks. (Two-way ANOVA, Bonferroni's post-hoc test). 5b-5d, Behavioral assessments using amphetamine-induced rotation test (5b), apomorphine-induced rotation test (5c), and cylinder test (5d) at 20 weeks post-transplantation. (One-way ANOVA, Tukey's post-hoc test; n=5 per group). 5e, 5f, The total number of transplanted C4-mDAPs (hNUCLEI⁺) (e) and graft volume (hNCAM⁺) (5f) were analyzed after 2 and 20 weeks, respectively. (Student's t-test; n=5 per group). 5g, 5h, Percentages of Ki67⁺ and SOX1⁺PAX6⁺Ki67⁺ cells (5g) and correlation between Ki67⁺ cell percentage and graft volume (5h). (Student's t-test; n=5 per group). 5i, The total number of TH⁺ cells were examined after 2 and 20 weeks, respectively. (Student's t-test; n=5 per group). 5j, Images of A9-like or A10-like cells. 5k, Quantification of TH⁺ neurons co-expressing GIRK2⁺ ALDHIA1⁺ (A9) or Calbindin⁺ (A10). (Student's t-test; n=5 per group). 5l, Percentage of NeuN⁺/hNUCLEI⁺ cells. (Student's t-test; n=5 per group). 5m, Images of DA synaptic connections within the host striatum identified by TH⁺hSYP⁻DARPP32⁺ staining. Each error bar represents means±s.e.m. ns, not significant; *, P<0.05; **, P<0.01; ***, P<0.001.

FIGS. 6a-6i: Influence of needle trauma-induced neuroinflammation on transplanted post-mitotic neurons. 6a-6e, NSG mice were sacrificed 14 days after intra-striatal transplantation of B1/H9-mDAPs. The total number and percentage of TH⁺ mDA neurons were determined by immunofluorescence staining with anti-TH/Hoechst 33342 before transplantation (6a, 6c, 6e) or immunohistochemistry staining with anti-TH/hNUCLEI antibodies after transplantation (6b, 6d, 6e). (n=4 per group). 6f-6i, NSG mice were sacrificed 14 days after intra-striatal transplantation of C4-iPSC/B1-iPSC/H9-ESC derived GABAergic progenitors. The total number and percentage of C4-(6f), B1-(6g), or H9-GABAergic neurons (6h) were determined by immunofluorescence staining with anti-VGAT/Hoechst 33342 before transplantation and anti-VGAT/Nkx2.1/hNUCLEI antibodies after transplantation (6i). (n=4 per group). Each error bar represents means±s.e.m.

FIGS. 7a-7g: Early time-dependent analysis of inflammatory responses to the needle trauma. 7a-7d, TP media was injected into the striatum of Fischer 344 rats that were sacrificed on Day 1, 2, 3, 5, 7 after surgery. The level of needle trauma-induced neuroinflammation was determined by immunofluorescence staining with anti-TNF-α (7a) and IL-1β (7b) antibodies, and staining is quantified in 7a′ and 7b′ (bar graphs in left panel, respectively). 7c, Astrocytes were stained with anti-GFAP antibody, and staining is quantified in 7c′ (bar graph in left panel). Images and quantitative analysis of Iba-1⁺ cells (yellow box: brain-resident microglia, red box: infiltrated Iba-1⁻ cells) (7d, 7d′) after injection. (n=5 per group). 7e-7g, Inhibition of needle trauma-induced host inflammatory response and cell death using T_{REG}. Fischer 344 rats were sacrificed 2 or 7 days after intra-striatal co-transplantation of TP medium with or without autologous T_{REG} from each rat (20 k cells/rat). The level of needle trauma-induced neuroinflammation was determined by immunofluorescence staining with anti-TNF-α (7e, 7e′) and IL-1β (7f, 7f′) antibodies. (Student's t-test; n=3 per group). 7g, 7g′, The level of needle trauma-induced neuronal cell death was determined by immunohistochemical staining with anti-NeuN antibody. (Student's t-test; n=5 per group). Each error bar represents means±s.e.m. *, P<0.05; **, P<0.01; ***, P<0.001.

FIGS. 8a-8d: Long-term time-dependent analysis of cellular responses to the needle trauma. 8a-8d, TP media was injected into striatum of Fischer 344 rats and rats were sacrificed on Day 4, 7, 14 and 1, 3, 6 months after surgery. The level of inflammatory cell infiltration into the needle track was determined by immunohistochemical staining with anti-Ibal (8a) and -MHCII (8b) antibodies. 8c, The level of needle trauma-induced neuroinflammation was determined by immunofluorescence staining with anti-IFN-γ antibody. 8d, The level of cell death by needle trauma was analyzed by TUNEL assay. (n=5 per group). The mean value was connected by a red line.

FIGS. 9a-9g: T_{REG} function after ex vivo expansion. 9a, 9b, The level of sorted CD4⁺CD25⁺Foxp3⁺ T cells (nT_{REG}) (9a) and ex vivo expanded rat CD4⁺CD25⁺Foxp3⁺ T cells (Expanded T_{REG}) (9b) were analyzed by flow cytometry staining. 9c, T_{REG} suppression assay of T_{conv} cells proliferation based on CFSE dilution in the presence of rat nT_{REG} or expanded T_{REG} at 1:1 and 1:2 ratio (T_{REG}: T_{conv}) and analyzed by flow cytometry. (Student's t-test; n=3). 9d-9f, Interaction between rat T_{REG} (rT_{REG}) and C4-mDAPs. C4-mDAPs and rT_{REG} were co-incubated for 72 hours at a ratio of 5:1 and 1:1. The level of TH (9d), Foxa2 (9e), or Lmx1a (9f) mRNA expression in C4-mDAPs were determined by quantitative real-time PCR and normalized to Actin. (One-way ANOVA, Tukey's post-hoc test; n=3 biologically independent experiments). (9g) The level of isolated human CD4⁺CD127^{low}CD25⁺Foxp3⁺ T_{REG} were analyzed by flow cytometry. Each error bar represents means±s.e.m. ns, not significant.

FIGS. 10a-10b: Localization and function of autologous T_{REG} after co-transplantation. 10a, 10b, After intra-striatal co-transplantation of Fischer 344 rats with TP medium and autologous T_{REG} (20 k cells/rat), (10a) the localization of T_{REG} was determined by immunofluorescence staining with anti-Foxp3 antibody at 1, 2, 3, 5, and 7 days after surgery. (One-way ANOVA, Tukey's post-hoc test; n=5 per group). 10b, The level of inflammatory cell infiltration into needle track was determined by immunohistochemical staining with anti-MHCII antibody. (Two-way ANOVA, Bonferroni's post-hoc test; n=5 per group). Each error bar represents means±s.e.m. **, P<0.01; ***, P<0.001.

FIGS. 11a-11e: Inhibition of needle trauma-induced neuroinflammation by CsA treatment. 11a-11e, Fischer 344 rats were sacrificed 7 days after intra-striatal co-transplantation of TP media with T_{REG} (20 k cells/rat) or CsA treatment (i.p.). 11a, Schematic overview of experimental method. The level of inflammatory cell infiltration into the needle track was determined by immunohistochemical staining with anti-Ibal (11b) and MHCII (11c) antibodies. 11d, The level of needle trauma-induced neuroinflammation was determined by immunofluorescence staining with anti-IFN-γ antibody. 11e, The level of neuroinflammation-induced cell death by needle trauma was analyzed by TUNEL assay. (One-way ANOVA, Tukey's post-hoc test; n=5 per group). Each error bar represents means±s.e.m. ns, not significant; *, P<0.05; **, P<0.01; ***, P<0.001.

FIGS. 12a-12p: In vitro effects of pro-inflammatory cytokines treatment on C4-mDAPs. 12a-12g, C4-mDAPs were incubated in vitro with or without 20 ng/ml TNF-α, 10 ng/ml IL-1β or 100 ng/ml IFN-γ for 2 days. The level of TNF-α/IL-1β-induced immunogenicity was determined by flow cytometry analysis with anti-HLA-ABC/HLA-DR/CD80/CD86/CD40/PD-L1/PD-L2/CD47 antibodies (12a) and by western blot analysis with anti-Zg16/Hormad1 antibodies (12b). TH levels were assessed by immunofluorescence staining (12c), the number of total cells (12d) or TH⁺ cells (12e), TH⁺ mean fluorescence intensity (12f), and TH⁺ neurite length (12g). (Student's t-test; n≥4). 12h, 12i, C4-mDAPs were incubated in vitro with 20 ng/ml TNF-α or 10 ng/ml IL-1β for 7 days. The level of TNF-α/IL-1β-induced cell death was determined by flow cytometry analysis of Annexin-V/7-AAD staining (12h) and TUNEL assay (12i). (One-way ANOVA, Tukey's post-hoc test; n=3 biologically independent experiments). 12j-12m, C4-mDAPs with or without T_{REG} were co-cultured for 7 days under in vitro inflammatory conditions (with or without 20 ng/ml TNF-α or 10 ng/ml IL-1β). 12j, Immunofluorescence staining of TH and FoxA2. Percentages of TH⁺ (12k) and FoxA2+ (12m) cells among total cells, and TH⁺neurite length (1) were measured. (One-way ANOVA, Tukey's post-hoc test; n≥3). 12n, 12n′, NSG mice were sacrificed 14 days after intra-striatal co-transplantation of C4-mDAPs with or without anti-IFN-γ mAb. The percentage of TH⁺mDA neurons was determined by immunohistochemistry staining with anti-TH/hNUCLEI antibodies after transplantation. (Student's t-test; n=5 per group). 12o, 12p, C4-mDAPs with or without T_{REG} and anti-TGF-β1 or mouse IgG1 isotype control antibody were co-cultured for 7 days. The level of proliferation was determined by immunofluorescence staining with anti-Ki67 antibody (12o) and staining is quantified in 12p. (One-way ANOVA, Tukey's post-hoc test; n=5). Each error bar represents means±s.e.m. ns, not significant; *, P<0.05; **, P<0.01; ***, P<0.001.

FIGS. 13a-13e: T_{REG} CO-transplantation suppresses inflammatory cell infiltration and C4-mDAPs immunogenicity in xenogeneic PD model. 13a-13e, Fischer 344 rats were sacrificed 2 weeks after intra-striatal co-transplantation of C4-mDAPs with or without T_{REG} and CsA. The levels of CD11b⁺/CD11c⁺ (13a), NKp46⁺ (13b), CD19⁺ (13c), and CD4⁺/CD8⁺ (13d, 13d′ (bottom panel)) cells were assessed through immunofluorescence staining. 13d′, Most of the yellow dots seen in the +T_{REG}, +CsA, and +T_{REG}+CsA groups in 13d are non-specific signals, not real cells (yellow arrows). (13e) Expression of HLA class I/II was examined through immunofluorescence staining. (n=5 per group).

FIGS. 14a-14k: T_{REG} co-transplantation suppresses C4-mDAPs proliferation in xenogeneic PD model. 14a-14k, Fischer 344 rats were sacrificed 20 weeks after intra-striatal co-transplantation of C4-mDAPs with or without T_{REG} and CsA. 14a, 14b, Stereological estimation of graft volume through hNCAM⁺ staining. Number of Ki67⁺ cells (14c, 14d) among hNUCLEI⁺ cells. (Student's t-test; n=5 per group). e, Correlation between Ki67⁺ cell number and graft volume. 14f, The number of TH⁺ and FoxA2⁺ cells were analyzed by immunofluorescence staining. (Student's t-test; n=20). 14g-14k, Images of neuronal (14g, NeuN⁺), astrocyte (14h, hGFAP⁺), VLMC (i, hCOL1A1⁺), oligodendrocyte (14j, OLIG2⁺), and microglia (14k, hIba-1⁺). Scale bars: 100 μm. Each error bar represents means±s.e.m. *, P<0.05; **, P<0.01; ***, P<0.001.

FIGS. 15a-15h: Effects of T_{REG} co-transplantation with C4-mDAPs in autologous C4-humanized PD model. 15a-15h, C4-humanized mice were sacrificed 8 weeks after intra-striatal co-transplantation of C4-mDAPs with or without autologous C4-T_{REG} (20 k cells/mouse). 15a, Schematic overview of experimental design. 15b, Survival of C4-humanized mice. Total number of transplanted C4-mDAPs (c), graft volume (15d), and TH⁺ cells (15g) were assayed. Number of Ki67″ (15e) (Student's t-test; n=5) and correlation between Ki67⁺ and hNUCLEI⁺ cell number (15f). 15h. The levels of hCD4⁺ T cells were confirmed through immunofluorescence staining. (Student's t-test; all data passed the Shapiro-Wilk normality test; n=2 (−T_{REG}) and n=3 (+T_{REG}) per group). Each error bar represents means±s.e.m. *, P<0.05.

FIGS. 16a-16d: In vivo effects of T_{REG} co-transplantation with C4-mDAPs. 16a-16d, NSG mice were sacrificed 20 weeks after intra-striatal co-transplantation of C4-mDAPs with or without autologous C4-T_{REG} (20 k cells/mouse). 16a, 16b, Images (16a) and quantitative assessment (16b) of hSYP⁺ within the DL STR. (Student's t-test; n=5 per group). 16c, 16d, Images (16c) and quantitative assessment (16d) of graft-derived TH⁺ fiber density in the (i) cingulate cortex (CTX), (ii) perirhinal CTX, (iii) DL STR, and (iv) ventrolateral (VL) STR. T, transplant. (Student's t-test; n=3 per group). Each error bar represents means±s.e.m. *, P<0.05; **, P<0.01; ***, P<0.00.

FIGS. 17a-17e: Functions of T_{REG} and related mechanisms. 17a-17c, The expression level of TGF-β receptor (17a), SIRPα (17b), and Galectin-1 (17c) in C4-mDAPs was analyzed by flow cytometry. 17d, 17e, NSG mice were sacrificed 8 weeks after intra-striatal co-transplantation of C4-mDAPs with mitomycin C-treated C4-T_{naïve} (20 k cells/mouse) or C4-T_{REG} (20 k cells/mouse). The total number of transplanted C4-mDAPs (hNUCLEI*) (17d) and graft volume (hNCAM⁺) (17e) were analyzed after 8 weeks, respectively. (Student's t-test; n=4 per group). Each error bar represents means±s.e.m. **, P<0.01.

FIG. 18: Dynamic profiles of molecular and cellular changes following needle trauma. Upon needle injection, the brain experiences physical damage, leading to the rupture of resident cells such as neurons, astrocytes, microglia, and oligodendrocytes. This rupture results from the impact of the needle, causing these cells to burst. Subsequently, the ruptured cells release damage-associated molecular patterns (DAMPs) rapidly, which affect neighboring cells and trigger the production and secretion of cytokines and chemokines due to activation. Among the cells sensing this response, neutrophils are quickly recruited to the damaged site, playing a pivotal role in promptly eliminating the debris. Concurrently, astrocytes and microglia become increasingly activated and migrate toward the damaged area over time. Approximately by the third day, peripheral monocytes infiltrate, and depending on the severity of the brain damage, T and B cells may also infiltrate, engaging in reparative functions. This sequence of inflammatory processes is crucial for the removal of cell debris resulting from needle trauma, facilitating essential steps for the repair and homeostasis of the damaged area. At the same time, however, this event appears to cause substantial damage and death to engrafted mDANs.

FIG. 19. Three different phases of hPSC-based CRT when mDANs may die. Schematic representation of the three phases in which mDANs can die. Phase 1 involves the in vitro differentiation of hPSCs into mDA cells, predominantly comprising mDAPs and mDANs, using optimized procedures. Phase 2 encompasses the harvesting and cryopreservation of in vitro differentiated mDA cells, emphasizing the critical steps of cryopreservation, storage, and thawing. In Phase 3, the final in vivo transplantation occurs, consisting of surgical transplantation, the early stage (<2 weeks), and the late stage (>2 weeks) of graft establishment. The potential challenges and considerations at each phase, including cell viability, immune responses, and environmental factors, are discussed for a comprehensive understanding of the optimization process in hPSC-based CRT for PD.

FIG. 20: Strategies targeting adaptive immunity. In general, allogeneic transplantation using mDA cells derived from hESCs still necessitates immunosuppression, whereas autologous transplantation with mDA cells from hiPSCs is characterized by immune tolerance, eliminating the need for immunosuppressants. An alternative strategy, distinct from utilizing autologous cells, involves the application of HLA-matched hiPSCs to reduce the risk of graft rejection, with ongoing efforts to establish an HLA-matched iPSC bank encompassing a diverse array of donors. Another approach focuses on the development of “universal donor stem cells,” incorporating genetic modifications, such as CRISPR/Cas9-mediated knockout of HLA class I and II components, along with lentiviral overexpression of the immune receptor CD47 or HLA-E/G transgene.

FIG. 21: Strategies targeting innate immunity. In CRT, the standard procedure involves the injection of cells into the brain using a needle, which induces needle trauma and subsequent secretion of various innate immune response factors, including DAMPs, pro-/anti-inflammatory cytokines, and chemokines. These factors activate surrounding glial cells and lead to the infiltration of peripheral immune cells into the brain, contributing positively to damage repair. However, they also exert a detrimental effect on grafted cells, resulting in severe cell death of mDANs. A potential strategy to enhance the survival of grafted cells involves obtaining autologous T_{REG} cells from the patient, increasing their quantity and functionality, and confirming improved therapeutic effects through co-transplantation into the brain. Additionally, exploring the efficacy of inhibitors, neutralizing antibodies, and encapsulation methods to enhance the survival of grafted cells within the inflammatory microenvironment, combined with the application of T_{REG} technology, represents a promising direction for future advancements in CRT.

FIG. 22: Antibodies targeting inflammatory cytokines decrease MHC-II⁺ cell infiltration. The level of inflammatory cell infiltration into needle track was determined by immunohistochemical staining with anti-MHCII antibodies.

### DETAILED DESCRIPTION

Parkinson's disease (PD) stands as the second most common neurodegenerative disorder after Alzheimer's disease, and its prevalence continues to rise with the aging global population. Central to the pathophysiology of PD is the specific degeneration of midbrain dopamine neurons (mDANs; mDA cells) in the substantia nigra. Consequently, cell replacement therapy (CRT) has emerged as a promising treatment approach, initially supported by various open-label clinical studies employing fetal ventral mesencephalic (fVM) cells. Despite the initial favorable results, fVM cell therapy has intrinsic and logistical limitations that hinder its transition to a standard treatment for PD. Recent efforts in the field of cell therapy have shifted its focus towards the utilization of human pluripotent stem cells, including human embryonic stem cells and induced pluripotent stem cells, to surmount existing challenges. However, regardless of the transplantable cell sources (e.g., xenogeneic, allogeneic, or autologous), the poor and variable survival of implanted dopamine cells remains a major obstacle. Emerging evidence highlights the pivotal role of host immune responses following transplantation in influencing the survival of implanted mDANs, underscoring an important area for further research. The present inventors investigated the functional ramifications of host immune responses on the survival and efficacy of grafted dopamine cells, and explored potential strategic approaches to modulate the host immune response, aiming for optimal outcomes in future clinical applications of CRT for PD.

Lessons from Fetal Ventral Mesencephalon (Fmv) Transplantation Studies

Clinical outcomes of fVM-based CRT varied widely, occasionally accompanied by graft-induced dyskinesia (GID)

Starting from the late 1980s, stem cell scientists attempted CRT using fVM tissues dissected from aborted embryos (typically 6 to 9 weeks old) because they showed the most promising outcomes among various dopamine-producing cells.⁶⁻¹³ In these early open-label trials using fVM tissues, some patients showed prominent and long-term improvement including enhanced movement, reduced rigidity and tremor; decreased ‘off’ periods; reduction of medication, or in a few cases, a complete cessation; and enhanced dopamine uptake, as evidenced by ¹⁸F-DOPA PET scan analyses (Table 1).¹⁴⁻³⁵ Subsequent postmortem studies of deceased transplanted patients further supported these positive clinical outcomes, revealing successful engraftment of implanted mDANs with robust outgrowth and innervation to the host striatum, demonstrating that engrafted mDANs were functionally integrated into neural circuits in the patient's brain. In summary, these successful clinical data provided the “proof of concept” of CRT in PD. Despite these positive results, subsequent double-blind, sham controlled studies showed clinical benefits were statistically insignificant and was not recommended as a treatment for PD.³⁶⁻³⁸ Furthermore, approximately 30% of transplanted patients (18 out of 56) developed a serious side effect: GID,³⁶⁻³⁸ significantly dampening the initial enthusiasm for the fetal cell-based approach. These inconsistent and disappointing outcomes, along with ethical, medical, and practical limitations, rendered this approach unfeasible as a standard treatment for PD.⁶⁻¹¹

The poor and variable survival rates of implanted mDANs may underlie the inconsistent clinical outcomes observed in fVM-based CRT

Why were clinical outcomes of fVM transplantation inefficient and variable? Over the last several decades, more than 400 PD patients have undergone fVM transplantation and have been thoroughly analyzed.⁶⁻¹³ Additionally, stem cell scientists conducted extensive pre-clinical studies using rat and mouse embryonic VM tissues with three objectives: (1) to understand inefficient and variable clinical outcomes of fVM transplantation, (2) to elucidate the biological factors influencing the survival of transplanted cells, and (3) to enhance the survival of implanted mDANs for future successful CRTs.³⁹⁻⁴¹ These clinical and pre-clinical studies revealed important insights and diverse potential factors contributing to inconsistent clinical outcomes. These factors encompass a range of variables, including the differing ages and clinical statuses of patients; varied fetal cell preparation methods; the heterogenous and variable status of fetuses (typically requiring 6 to 8 fetuses per patient); variable immune suppression regimens; and target sites of transplantation (putamen, caudate, and/or the substantia nigra) (Table 1).^{6-14,18-31,35,37,38,42} While all these donor- and/or host-specific factors likely play important roles in clinical outcomes, we speculate that the poor and variable survival of transplanted mDANs in the graft directly cause inefficient clinical outcomes. Indeed, this survival issue of grafted mDANs has been a focal point of CRT research since the conception of fVM transplantation due to the limited supply of aborted fetuses. As outlined in an insightful review by Brundin and colleagues,⁴³ the survival of grafted mDANs in 31 independent studies using rat embryonic VM transplantation ranged between 0.7 and 23.3% with an average of 6.86%. Similarly, a consistent survival range between 5 and 10% was reported in transplantation studies of human VM grafts in athymic rats^{44,45} as well as in postmortem studies of human fVM transplantation.^{9,12,13,22,23,27,37,38,42} In conclusion, the pivotal lesson from previous clinical and pre-clinical studies is the highly inefficient and variable survival of grafted mDANs, which likely constitute the root cause of the poor and inconsistent clinical outcomes observed.

TABLE 1
List of Clinical Trials Using Human Fetal Tissue

| | | | Number | | | | | | | | |
|---|---|---|---|---|---|---|---|---|---|---|---|
| | Average | | of | | | | | | | | |
| Number | age of | Disease | fVM/ | | Immuno- | Follow- | | | | | |
| of | cohort | duration | side | | suppression | up | | TH⁺ cell | | Adverse | |
| patients | (range) | (years) | (age) | Grafted site | (period) | period | | survival | Outcome | effect | Ref. |
| 2 | 51.5 | 7 | 3-4 | Unilateral | CsA, AZA, | 2 | yrs | N/A | Minor | None | 14, |
| | (48-55) | | (8-10 | (C + P) | PSL | | | | improvement of | | 24 |
| | | | wks) | | | | | | motor speed for | | |
| | | | | | | | | | pronation- | | |
| | | | | | | | | | supination | | |
| 2 | 55 | 8-11 | 4 | Unilateral (P) | CsA, AZA, | 10 | yrs | n = 1 | Improved rigidity | None | 25, |
| | (50-60) | | (6-7 | | PSL (64 mo) | | | 6,246 (rostral); | and | | 26 |
| | | | wks) | | | | | 10,728 (middle); | bradykinesia | | |
| | | | | | | | | 25,854 (caudal) | | | |
| 7 | 56.3 | 7-20 | 1 | 2 pts- | CsA, PSL | 1-4 | yrs | N/A | Motor | None | 18 |
| | (39-66) | | (7-8 | unilateral(C + P); | in 4 pts | | | | improvement; | | |
| | | | wks) | 5 pts-bilateral (P) | | | | | Shorter and less | | |
| | | | | | | | | | severe | | |
| | | | | | | | | | dyskinesia | | |
| 2 | 36.5 | 6 | 3-4 | Bilateral (C + P) | CsA (12 mo), | 2 | yrs | N/A | Improved motor | None | 31 |
| | (30-43) | | (6-8 | | AZA (18 mo), | | | | functions | | |
| | | | wks) | | PSL (N/A) | | | | | | |
| 4 | 52 | 10-21 | 1 | Unilateral (C) | CsA (6 mo) | 1.5 | yrs | N/A | Improved motor | Transient panic | 29 |
| | (47-63) | | (7-11 | | | | | | functions (3/4) | disorder (1/4) | |
| | | | wks) | | | | | | | | |
| 2 | 56 | 10-17 | 2-3 | 1 pt-unilateral | CsA (7 mo), | 1.5 | yrs | N/A | Improved motor | Transient | 17 |
| | (49-63) | | (6-9 | (P); | AZA, PSL | | | | functions | obsessive | |
| | | | wks) | 1 pt-bilateral | | | | | | compulsive | |
| | | | | (C + P) | | | | | | disorder (1/2) | |
| 4 | 52.3 | 8-22 | 3-4 | Bilateral (P) | CsA (6 mo) | 1.5 | yrs | Patient 1 | Improved motor | Cortical | 19, |
| | (39-61) | | (6.5-9 | | | | | (18 mo): | functions; | hemorrhage | 22, |
| | | | wks) | | | | | 128,162 (right), | Resolution of | (1/4); | 23 |
| | | | | | | | | 81,905 (left); | dyskinesias | Transient | |
| | | | | | | | | Patient 2 | | confusion and | |
| | | | | | | | | (19 mo): | | hallucinations | |
| | | | | | | | | 79,207 (right), | | (1/4) | |
| | | | | | | | | 39,151 (left) | | | |
| 5 | 57.4 | 13-22 | 1-3 | 1 pt-unilateral | CsA (7 mo), | 3 | yrs | N/A | Modest | None | 15 |
| | (48-67) | | (6-9 | (P); | AZA, PSL | | | | improvement in | | |
| | | | wks) | 4 pts-unilateral | | | | | daily living | | |
| | | | | (C + P) | | | | | activities (1/5) | | |
| 6 | 48.8 | 7-16 | 4-8 | 4 pts- | CsA, AZA, | ≤18 | yrs | N/A | Major | Atypical | 20, |
| | (43-58) | | (6-8 | unilateral (P); | PSL | | | | improvement | parkinsonism | 21, |
| | | | wks) | 1 pt- | | | | | (4/6); | (1/6); | 30 |
| | | | | unilateral (C + P) | | | | | Modest changes | Dementia | |
| | | | | | | | | | (2/6) | (1/6) | |
| 20 | ≤60 yrs: | ≤60: | 2 | Bilateral (P) | None | 3 | yrs | Patient 1 (7 mo): | Improved | Dystonia and | 37 |
| | 50 ± 8; | 13 ± 3; | (7-8 | | | | | 24,115 (right), | UPDRS score in | dyskinesia | |
| | >60 yrs: | >60: | wks) | | | | | 38,392 (left); | younger | (5/20) | |
| | 65 ± 4 | 15 ± 6 | | | | | | Patient 2 | pts(≤60 yrs), | | |
| | | | | | | | | (36 mo): | but not in older | | |
| | | | | | | | | 36,796 (right), | pts(>60 yrs) | | |
| | | | | | | | | 6,840 (left) | | | |
| 3 | 53 | 10-14 | 6 | Bilateral | CsA (6 mo) | 1.1 | yrs | N/A | Improved | None | 16 |
| | (48-59) | | (6-9 | (P + SN) | | | | | UPDRS score; | | |
| | | | wks) | | | | | | Decreased L- | | |
| | | | | | | | | | DOPA dose | | |
| | | | | | | | | | requirement | | |
| | | | | | | | | | (1/3) | | |
| 23 | 58.5 | 11 | 1-4 | Bilateral (P) | CsA (6 mo) | 2 | yrs | 1 fVM (n = 2): | No improvement | Dyskinesia | 38 |
| | (30-75) | | (6-9 | | | | | 30k/side; | | (13/23) | |
| | | | wks) | | | | | 4 fVM (n = 2): | | | |
| | | | | | | | | 70k-120k/side | | | |
| 2 | 64 | 11-15 | 2-3 | Bilateral (P) | CsA (6 mo) | 3 | yrs | Patient 1 | Improved | None | 27 |
| | (59-69) | | (6-9 | | | | | (44 mo): | UPDRS and | | |
| | | | wks) | | | | | 127,189 (right), | Dyskinesia | | |
| | | | | | | | | 98,913 (left); | scores | | |
| | | | | | | | | Patient 2 | | | |
| | | | | | | | | (52 mo): | | | |
| | | | | | | | | 202,933 (right), | | | |
| | | | | | | | | 4,289 (left) | | | |

Abbreviations: fVM, fetal ventral mesencephalic; yrs, years; wks, weeks; mo, months; C, Caudate; P, Putamen; SN, Substantia Nigra, CsA, Cyclosporin A; AZA, Azathioprine; PSL, Prednisolone; pts, patients; N/A, not available; UPDRS, Unified Parkinson’s Disease Rating Scale; Ref., Reference.

When and why do grafted mDANs die during fVM transplantation?

As described above, the survival of transplanted mDANs was extensively investigated in preclinical studies using rodent embryonic VM cells to model human fVM transplantation.^{43,46-52} A salient feature of these studies is that a great majority of mDANs die shortly after transplantation, typically within one week, and the number of surviving mDANs in the graft does not increase or change at later time points. This observation is rather surprising considering that embryonic VM cells contain a significant number of early progenitor cells in addition to already differentiated mDANs. These data suggest two possibilities: (1) progenitor cells in fVM have very limited capacity for proliferation and differentiation and/or (2) the host environment does not support their proliferation and differentiation into mDANs. Based on these pre-clinical studies, Brundin and colleagues proposed four distinct phases during which mDANs may die.⁴³ In Phase 1 (removal of embryos), mDANs may die of hypoxic and hypoglycemic insults that occur during embryo removal from maternal blood supply. In Phase 2 (cell preparation), mDANs may die of axotomy and other traumatic damages caused by mechanical dissociation. In Phase 3 (intrastriatal injection), mDANs may die during the implantation procedure and the immediate period following graft injection, and in Phase 4 (graft maturation), mDANs may die during maturation and innervation in the host brain.

Extensive studies have been conducted to understand the poor survival of grafted mDANs and the reasons for their early death post-transplantation, revealing diverse possibilities. When mDANs, dissected from embryos, are transplanted into the striatum under PD conditions, they become deprived of nutrients and growth factors crucial for their sustenance. Aligning with this concept, pre-treatment of cells with growth factors like basic fibroblast growth factor (bFGF) and glial cell line-derived neurotrophic factor (GDNF) has significantly enhanced the survival of grafted mDANs. Notably, while bFGF pre-treatment resulted in an approximately 2-fold increase in survival rate,^{53,54} continuous delivery through co-transplantation of bFGF-overexpressing fibroblasts led to a 10-fold increase, fostering more robust and rapid behavioral recovery.⁵⁵ In addition, since neuronal injury is often associated with excitotoxicity, oxidative stress, and calcium imbalance, researchers have explored pathways and molecules associated with these phenomena. Among these, calcium channel blockers (such as flunarizine) and lipid peroxidation inhibitors (like lazaroids) have shown notable effects on mDAN survival.⁵⁶⁻⁵⁸ Additionally, inhibition of cell death pathways, such as caspase inhibitors (e.g., Ac-YVAD-cmk) significantly enhanced mDAN survival.⁵¹

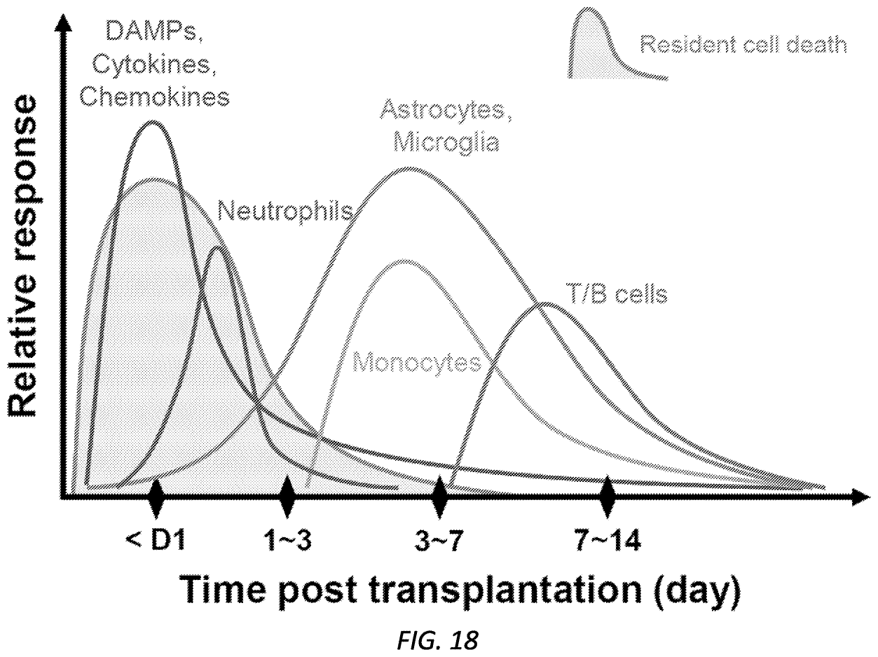

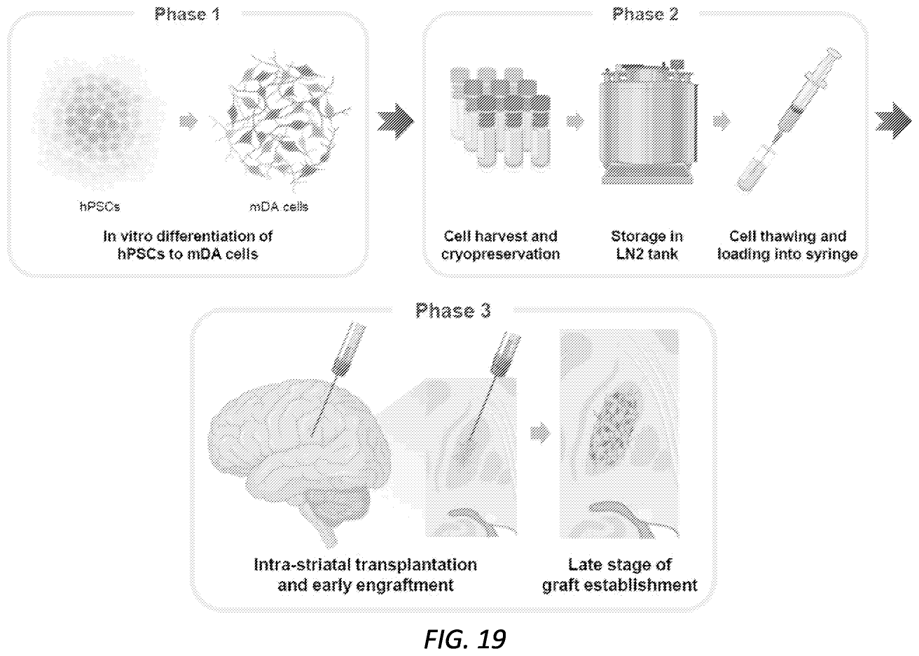

When do hPSC-derived mDANs die during the transplantation process? A major advantage of hPSCs lies in their potential to produce unlimited amounts of transplantable cell sources, such as mDANs. Possibly due to this advantage, cell survival in hPSC-based CRT, unlike fVM-based CRT, has not garnered sufficient attention and remains less investigated. Given the significant differences in cellular, developmental, and proliferation properties between fVM and hPSCs, both similar and distinct factors must be considered for successful CRT in PD. To address these issues, we investigated the cell survival issue of hPSC-based CRT using several rodent models such as wild type (with immunosuppression) and athymic rats as well as immunocompromised NOD SCID gamma (NSG) mice and humanized NSG mice.⁸⁴ Interestingly, the majority (approximately 90%) of mDANs (derived from both hESCs and hiPSCs) died within the first 1-2 weeks after transplantation, which is very similar to fVM transplantation.^{43,46-52} In addition, this study revealed that transplantation procedure itself triggered acute host inflammatory response. Remarkably, the host immune response was triggered even when only media was injected without any cell, indicating that this is induced by the host innate immune response. Because the immune response pattern was very similar to that of traumatic brain injury (TBI) (FIG. 18),^{87,88} we termed this phenomenon “needle trauma”0.84 This needle trauma appears to physically damage the host brain, leading to acute cell death of host neuronal cells around the injection path, which is thought to trigger subsequent immune responses such as immediate secretion of proinflammatory cytokines (e.g., TNF and IL-1β), activation of astrocytes/microglia, and robust infiltration of Iba-1+ and major histocompatibility complex (MHC)II⁺ inflammatory cells near the needle track, which peaked at day 7 before declining at 1 month and disappearing at 6 months. Importantly, this needle trauma preferentially led to the death of most mDANs, rather than midbrain dopamine progenitors (mDAPs), within the graft. Furthermore, unlike fVM-based CRT, the total number of mDANs (and grafted cells) significantly increased during the later stage, suggesting that some mDAPs proliferated and differentiated into new mDANs in the host brain post-transplantation.⁸⁴ In light of these new findings, coupled with insights from previous fVM transplantation studies,^{43,46-52} it appears that hPSC-derived mDANs died during one of three phases in the hPSC-based CRT procedure (FIG. 19).

In Phase 1, hESCs/hiPSCs are cultured and subsequently differentiated in vitro over a specific period using optimized procedures (FIG. 19). While various laboratories employ diverse optimized protocols,^{65,89-96} these methods typically use dual inhibition of SMADs, targeting BMP and TGFb signaling, and dual activation of WNT and SHH signaling, based on previous developmental studies. This in vitro differentiation process leads to the generation of mDA cells, predominantly comprising mDAPs and mDANs, in the range of 60-95% and 5-25%, respectively, depending on individual protocols.^{65,89-96} These protocols vary in detailed methods and media components. For instance, some protocols utilize a 2D monolayer method, while others employ a combination of 2D and 3D cultures, such as embryoid body and floating neurosphere cultures. There is a possibility that mDA cells differentiated in vitro may lose viability during the differentiation process. For example, we observed that a significant portion of cells die and/or become apoptotic on evenly distributed monolayer culture and that dividing the monolayer into smaller isolated portions, known as “spotting method” as described previously (e.g., WO 2020/237104), significantly reduced the percentage of these unhealthy or apoptotic cells during in vitro differentiation process.^{65,97} Phase 1 is analogous to the initial step of fVM-based CRT, where donor embryos are dissected and prepared as either cell suspension or tissue blocks for transplantation.⁴³ Previous studies demonstrated that the cell viability was substantially affected by different conditions and types of the grafting medium used for preparation of fVM-derived cells.⁹⁸-100 Therefore, in vitro differentiation conditions and culture media should be rigorously tested and optimized to maximize the viability. This optimization is crucial as the non-viable/apoptotic component of these final cell product will persist until transplantation and could negatively impact on clinical outcomes. For instance, in conjunction with needle trauma-induced neuroinflammation, this dead or apoptotic component of transplanted cell product may trigger an additional host immune response in Phase 3. Although one can design a FACS procedure to exclude dead/dying cells, it may impose additional harmful effects on viable cells.

In Phase 2, in vitro differentiated mDA cells are harvested and cryopreserved in liquid nitrogen until transplantation (FIG. 19). Recent studies indicate that hPSC-derived mDA cells can be cryopreserved without losing their viability, cellular phenotypes, and in vivo function.^{65,93,101-103} Consequently, most groups are planning to incorporate this cryopreservation step in hPSC-based CRT. Phase 2 comprises three key steps: (i) harvesting and cryopreserving the in vitro differentiated mDA cells using an optimal cryopreservation medium in cryovials, (ii) storing multiple cryovials in liquid nitrogen, and (iii) thawing the cryovials and loading mDA cells into a surgical syringe before transplantation. In theory, mDA cells can undergo cell death at any of these steps. For example, although hPSC-derived mDA cells have been reported to maintain their viability and function, the duration for which they can be stored in liquid nitrogen without compromising their viability and function remains an area of uncertainty. Given that needle trauma has been observed to preferentially affect mDANs more than mDAPs, it is plausible that the impact of cryopreservation and subsequent handling varies significantly depending on cell types.⁸⁴ Therefore, revisiting this issue and systematically analyzing the viability of different cell types at each step could be crucial for the final optimization of the process.

Phase 3 constitutes the final in vivo transplantation step and encompasses (i) surgical transplantation, (ii) the early stage (<2 weeks), and (iii) the late stage (>2 weeks) of graft establishment (FIG. 19). Host immune responses operate at these steps and may lead to the death of mDA cells. During the initial step of graft injection, mDA cells may retain viability in the syringe for a limited period (e.g., 10-20 min). However, prolonged delays during the injection process, exceeding the norm in clinical procedures, may result in the death of a portion of mDA cells (especially mDANs) even before they are injected into the host brain. Thus, minimizing delay in the surgical procedure is advisable. Notably, the surgical procedure itself appears to trigger acute innate immune responses, culminating in a hostile neuroinflammation environment. Injected mDANs are consequently subjected to early and preferential cell death. Furthermore, adaptive immune response between grafted cells and the host immune system may occur, potentially leading to graft rejection in absence of immunosuppression. In the later stage of engraftment in Phase 3, surviving mDA cells undergo maturation, differentiation, and integration into the host brain to establish new functional circuits. However, it is plausible that a portion of new and/or maturing mDANs may succumb even at this late stage due to various factors such as continued host immune responses, insufficient essential factors (e.g., blood supply, growth factors, and oxygen), and an unfavorable PD environment characterized by neuroinflammation, oxidative stress, and a-synucleinopathy.

When human induced pluripotent stem cell (hiPSC)-derived mDA cells were transplanted into the striatum of immunodeficient NOD SCID gamma or humanized mice, only a small percentage (<10%) of implanted tyrosine hydroxylase (TH)⁺ mDA neurons survived 2 weeks post-transplantation. In contrast, most of the remaining, TH grafted cells survived. Remarkably, transplantation of autologous regulatory T cells (T_{REG}) greatly modified the response to needle trauma caused by the transplantation of the mDA cells, suppressing acute neuroinflammation and immune cell infiltration. Furthermore, intra-striatal co-transplantation of T_{REG} and hiPSC-derived mDA cells significantly protected grafted mDA neurons from needle trauma-associated death and substantially improved therapeutic outcomes in 6-OHDA lesioned PD rodent models. Co-transplantation with T_{REG} also suppressed undesirable proliferation of TH grafted cells, resulting in more compact grafts with a higher proportion and higher absolute numbers of TH⁺neurons. Taken together, these data emphasize the importance of the initial inflammatory response to surgical injury in differential survival of cellular components of the graft and suggest that co-transplantation of autologous T_{REG} with iPSC-derived mDA grafts effectively reduces needle trauma-induced death of mDA neurons.

### Methods of Treatment

Thus, the present methods provide clinically applicable personalized cell therapy for PD that increases the viability of the grafted cells. In some embodiments, described herein are methods of treating PD, methods of increasing cell survival in a cell transplantation, methods of increasing outgrowths of transplanted cells, and/or methods of reducing overgrowth during a cell transplantation.

As needle trauma is likely to be a significant factor in cell therapy success for all cell types, described herein also are methods of increasing graft viability of a cell therapy and/or decreasing the immune reaction in a subject receiving a cell therapy.

In some embodiments, the methods described herein comprise administering to a subject in need thereof (e.g., a subject having a cell therapy and/or a subject having (or at risk of developing) PD) an effective amount of a population of T_{REG} cells with a second population of cells (e.g., mDA cells).

In some embodiments, the methods described herein comprise administering to a subject in need thereof (e.g., a subject having a cell therapy and/or a subject having (or at risk of developing) PD) an effective amount of an antibody (Ab) (e.g., an antibody that binds a pro-inflammatory cytokine or a damage-associated molecular patterns (DAMPs) molecule; optionally an anti-TNF-α Ab (e.g., Infliximab), an anti-IL-1β Ab (e.g., Canakinumab), an anti-IFN-γ Ab (e.g., Emapalumab), an anti-HMGB1 Ab, and an anti-IL-1α Ab) with a second population of cells (e.g., mDA cells).

In some embodiments, the methods described herein comprise:
the administration of a population of T_{REG} cells;
the administration of a population of mDA cells;
the administration of an Ab that binds a pro-inflammatory cytokine (e.g., an anti-TNF-α Ab, an anti-IL-1β Ab, an anti-IFN-γ Ab);
the administration of an Ab that binds a damage-associated molecular pattern (DAMPs; e.g., an anti-HMGB1 Ab, an anti-IL-1α Ab);
the administration of a composition comprising a population of T_{REG} cells and a second population of cells (e.g., mDA cells); and/or
the administration of a composition comprising an Ab that binds a pro-inflammatory cytokine (e.g., an anti-TNF-α Ab, an anti-IL-1β Ab, an anti-IFN-γ Ab) or an Ab that binds a DAMPs molecule (e.g., an anti-HMGB1 Ab, an anti-IL-1α Ab), and a population of cells (e.g., mDA cells); to a subject in need thereof.

Such subjects (e.g., a subject receiving a cell therapy, as subject identified as benefiting from a cell therapy, a subject having PD, or a subject at risk of developing PD) can be identified by skilled healthcare providers using methods known in the art.

Populations of T_{REG} cells useful in the present methods are described herein and known in the art. Useful T_{REG} cells can express one or more of FoxP3, CD25, CD47, CD45, CD4, CD8, and CD127. See also US 2023/0323299, US 2023/0047159, US 2020/0330515, and references 163-167. Thus, the methods herein can include obtaining a sample (e.g., blood or PBMC cells) comprising T cells from a subject, isolating Treg cells, expanding the Treg cells, and administering the cells. In some embodiments, the primary somatic cells are obtained from the subject to be treated (e.g., a subject who has (or is at risk of developing) PD), but in some embodiments the cells are obtained from a different subject, in some embodiments, a subject of the same species as the subject who is to be treated, preferably an immunologically matched subject.

The methods described herein can include the use of induced pluripotent stem cells (hiPSCs), e.g., similar to neurogenic floor plate cells, which can be generated using methods known in the art or described herein. In some embodiments, the methods for generating hiPSC can include obtaining a population of primary somatic cells from a subject, e.g., a subject undergoing a cell therapy, a subject who is afflicted with PD, a subject who is at risk of developing PD, and/or a subject in need of treatment for PD. Preferably the subject is a mammal, e.g., a human.

The methods can include obtaining primary somatic cells; generating a population of cells comprising mDA cells, and administering the cells. In some embodiments, the primary somatic cells are obtained from the subject to be treated (e.g., a subject who has (or is at risk of developing) PD), but in some embodiments the cells are obtained from a different subject, in some embodiments, a subject of the same species as the subject who is to be treated, preferably an immunologically matched subject. Preferably the mDA cells are generated by a method as described herein sufficient to generate a population comprising cells that express one, two, or more mDAP markers (e.g., FOXA2, OTX2, LMX1A, and EN1, e.g., FOXA2 and LMX1A; optionally TH⁺ cells that co-express FOXA2, LMX1A and NURR1), and optionally comprising cells that express one, two, or more mDAN markers (e.g., TH, DAT, and PITX3), but not comprising cells that express SOX1, PAX6, and KI67.

Populations of mDA cells useful in the present methods are described herein and known in the art. A population of mDA cells useful for the methods described herein can be derived from hESCs/hiPSCs and subsequently differentiated in vitro over a specific period using optimized procedures (FIG. 19).^{65,89-96} In some embodiments, a population of mDA cells comprises mDAPs and mDANs. In some embodiments, a population of mDA cells comprises at least about 50, 55, 60, 65, 70, 75, 80, 85, 90, or 95% mDAPs and at least about 5, 10, 15, 20, or 25% mDANs. See also US 2022/0243174; US 2018/0371422; US 2012/0128655; US 2013/0052268; US 2016/0002604; US 2014/0199274; and US 2009/0226401; as well as U.S. Pat. Nos. 11,898,169; 11,001,809; 9,657,273; and 9,750,768; and references 61-65 and 89-96.

In some embodiments, the methods described herein decrease an immune response in a subject triggered by needle trauma during a cell transplantation and or treat PD. In some embodiments, the methods described herein increase the number and/or percentage of TH⁺ cells in a graft (e.g., as compared to a graft without co-transplanting a population of T_{REG} cells or the Ab targeted to a pro-inflammatory cytokine). In some embodiments, the methods described herein increase in the ratio of TH⁺ cells:TH⁻ cells in the graft and/or an increase in the TH⁺ cells:Ki67+ cells ratio in a graft (e.g., as compared to a graft without co-transplanting a population of T_{REG} cells or the Ab targeted to a pro-inflammatory cytokine). In some embodiments, the methods described herein decrease the number and/or percentage of Ki67⁺ cells in the graft and/or a decrease the number and/or percentage of TH⁻ cells in a graft (e.g., as compared to a graft without co-transplanting a population of T_{REG} cells or the Ab targeted to a pro-inflammatory cytokine).

In some embodiments, the cells, antibodies, and compositions described herein can be administered to a subject described herein. In some embodiments, the administering step of any of the methods described herein comprises systemic, parenteral, intravenous, cerebral, cerebrospinal, intrathecal, intracisternal, intraputaminal, intrahippocampal, intra-striatal, or intra-cerebroventricular administration. In some embodiments, the administering step comprises intravenous, cerebral, cerebrospinal, intrathecal, intracisternal, intraputaminal, intrahippocampal, intrastriatal, or intracerebroventricular injection. In some embodiments, the administering step comprises direct injection into the pars compacta of the substantia nigra of the brain. In some embodiments, the administering step comprises introducing the cell therapy and/or antibody directly into the subject's brain or cerebrospinal fluid (CSF).

In some embodiments, the cells, antibodies, and compositions can be administered using methods known in the art. In some embodiments, the cells are administered by being implanted directly into or near the affected area of the subject's brain, e.g., bilaterally or unilaterally into one or more of the caudate nucleus, putamen, and substantia nigra, e.g., using magnetic resonance imaging-guided stereotactic surgery. See, e.g., Garitaonandia et al., Stem Cells Dev. 2018 Jul. 15; 27(14):951-957; Kikuchi et al., Nature 548:592-596 (31 Aug. 2017); MOrizane et al., Nature Communications 8:385 (2017); Sonntag et al., Prog Neurobiol. 2018 September; 168:1-20.

In some embodiments, the cells, antibodies, and compositions described herein can be administered in a single dose or repeat doses.

In some embodiments, the T_{REG} cells and the transplanted cells (e.g., mDA cells) are administered to the subject at the same time. In some embodiments, the T_{REG} cells and the transplanted cells (e.g., mDA cells) are administered separately. In some embodiments, the T_{REG} cells are first administered to the subject and subsequently the transplanted cells (e.g., mDA cells) are administered to the subject. In some embodiments, the transplanted cells (e.g., mDA cells) are first administered to the subject and subsequently the T_{REG} cells administered to the subject.

In some embodiments, the antibody (Ab) (e.g., an antibody that binds a pro-inflammatory cytokine or a damage-associated molecular patterns (DAMPs) molecule; optionally an anti-TNF-α Ab (e.g., Infliximab), an anti-IL-1β Ab (e.g., Canakinumab), an anti-IFN-γ Ab (e.g., Emapalumab), an anti-HMGB1 Ab, an anti-IL-1α Ab) and the transplanted cells (e.g., mDA cells) are administered to the subject at the same time. In some embodiments, the antibody (Ab) (e.g., an antibody that binds a pro-inflammatory cytokine or a damage-associated molecular patterns

(DAMPs) molecule; optionally an anti-TNF-α Ab (e.g., Infliximab), an anti-IL-1β Ab (e.g., Canakinumab), an anti-IFN-γ Ab (e.g., Emapalumab), an anti-HMGB1 Ab, an anti-IL-1α Ab) and the transplanted cells (e.g., mDA cells) are administered separately. In some embodiments, the antibody (Ab) (e.g., an antibody that binds a pro-inflammatory cytokine or a damage-associated molecular patterns (DAMPs) molecule; optionally an anti-TNF-α Ab (e.g., Infliximab), an anti-IL-1β Ab (e.g., Canakinumab), an anti-IFN-γ Ab (e.g., Emapalumab), an anti-HMGB1 Ab, an anti-IL-1α Ab) is first administered to the subject and subsequently the transplanted cells (e.g., mDA cells) are administered to the subject. In some embodiments, the transplanted cells (e.g., mDA cells) are first administered to the subject and subsequently the antibody (Ab) (e.g., an antibody that binds a pro-inflammatory cytokine or a damage-associated molecular patterns (DAMPs) molecule; optionally an anti-TNF-α Ab (e.g., Infliximab), an anti-IL-1β Ab (e.g., Canakinumab), an anti-IFN-y Ab (e.g., Emapalumab), an anti-HMGB1 Ab, an anti-IL-1α Ab) is administered to the subject. In some embodiments, the antibody and cells are administered to the patient intrastriatally. In some embodiments, the cells are administered to the subject using one administration method (optionally, intrastriatally) and the antibody is administered to the subject using a different administration method (optionally, intravenously).

In some embodiments, the T_{REG} cells are autologous T_{REG} cells, allogenic T_{REG} cells, or xenogeneic T_{REG} cells. In some embodiments, the transplanted cells (e.g., mDA cells) are autologous mDA cells, allogenic transplanted cells (e.g., mDA cells), or xenogeneic transplanted cells (e.g., mDA cells). In some embodiments, the T_{REG} cells are transplanted intrastriatally. In some embodiments, the transplanted cells (e.g., mDA cells) are transplanted intrastriatally.

Compositions comprising an antibody (Ab) (e.g., an antibody that binds a pro-inflammatory cytokine or a damage-associated molecular patterns (DAMPs) molecule; optionally, an anti-TNF-α Ab (e.g., Infliximab), an anti-IL-1β Ab (e.g., Canakinumab), an anti-IFN-γ Ab (e.g., Emapalumab), an anti-HMGB1 Ab, an anti-IL-1α Ab) and methods of administering said compositions are well known in the art.

In some embodiments, the administering step comprises systemic, parenteral, intravenous, cerebral, cerebrospinal, intrathecal, intracisternal, intraputaminal, intrahippocampal, intra-striatal, or intra-cerebroventricular administration. In some embodiments, the administering step comprises intravenous, cerebral, cerebrospinal, intrathecal, intracisternal, intraputaminal, intrahippocampal, intra-striatal, or intra-cerebroventricular injection. In some embodiments, the administering step comprises direct injection into the pars compacta of the substantia nigra of the brain. In some embodiments, the administering step comprises introducing the cell therapy and/or antibody directly into the subject's brain or cerebrospinal fluid (CSF).

See also US 2022/0243174; US 2018/0371422; US 2012/0128655; US 2013/0052268; US 2016/0002604; US 2014/0199274; and US 2009/0226401; as well as U.S. Pat. Nos. 11,898,169; 11,001,809; 9,657,273; and 9,750,768.

Needle trauma is known in the art, but it is particularly concerning for cell therapies, which require a needle with a larger diameter to administer without clogging the needle or damaging the cellular therapy. Needle characteristics include inner and outer diameters, length, stiffness and bevel design. Deep subcortical target structures, such as the caudate nucleus or corpus striatum, require a long, thin needle/cannula (typically 19 cm or more) of sufficient rigidity to penetrate to the target site without injuring the overlying structure. Shorter needles (8-10 cm) would require direct brain exposure, which is more invasive (Amer, M. H., et al. (2017) Translational considerations in injectable cell-based therapeutics for neurological applications: concepts, progress and challenges. npj Regen Med 2, 23; Kondziolka, D., et. al. (2011) Injection parameters affect cell viability and implant volumes in automated cell delivery for the brain. Cell Transplant. 20, 1901-1906).

The needles used to administer a cell transplant can be at least 30G, 28G, 26G, 24G, 22G, 20G, 18G or larger and/or have outer dimensions (O.D.) of at least about 0.3, 0.4, 0.5, 0.6, 0.7, 0.8, 0.9, 1.0, 1.1, 1.2, 1.3, 1.4, and 1.5 mm, including graduated outer diameter between any of these parameters (e.g., 1.5 to 0.5 mm).

Needle parameters are known in the art and can be determined by a skilled healthcare professional using methods known in the art. See also,
Song et al., JCI 2020, Park et al., Nature 2023: 26G, Outer Dimension (O.D.), 0.464 mm;
Doi et al., Nature Communication 2020: 22G, O.D., 0.718 mm and 24G, O.D., 0.566 mm;
Kriks et al., Nature 2011: 28G, 0.362 mm;
Schweitzer et al., NEJM 2020: 22G, O.D.; 0.72 mm;
Freed et al., NEJM 2001: The tissue implants were placed with the use of a stainless-steel guide cannula with a graduated outer diameter of 1.5 to 0.6 mm. A round-ed stylet was contained in the bore of the cannula during its passage to the posterior tip of the putamen;
Spencer et al., NEJM 1992:1 mm; and Freed et al., NEJM 1992: A 1.5-mm cannula extended from its support in the stereotactic apparatus to the surface of the brain, where an inner stylet with an outer diameter of 0.46 or 0.64 mm penetrated the putamen.
   Upon needle injection, the brain experiences physical damage, leading to the rupture of resident cells such as neurons, astrocytes, microglia, and oligodendrocytes. This rupture results from the impact of the needle, causing these cells to release damage-associated molecular patterns (DAMPs) rapidly, which affect neighboring cells and trigger the production and secretion of cytokines and chemokines due to activation.

The data presented herein show for the first time that this needle trauma is directly responsible for the death of host brain cells near the injection site and a significant increase in inflammation, which is a substantial hurdle to the success in a cell therapy (e.g., graft success in a cell transplantation).

### Host Immune Responses in Hpsc-Based Crt

The survival of engrafted mDA cells is significantly influenced by various levels of host immune responses. A fundamental requirement for the survival of transplanted cells is to evade graft rejection, a process regulated by the host's adaptive immune system. Traditionally, certain specialized organs such as the brain and the eye have been considered immune-privileged,¹⁰⁴ which might explain why an earlier fVM transplantation study did not use immunosuppressants (Table 1). Conversely, an opposing viewpoint suggests that even autologous cells can elicit an immune response if differentiated cell products express immunogenic antigens.^{83,105} To investigate this contentious issue of immunogenicity in autologous vs. allogeneic intracerebral grafts, we conducted transplantation experiments involving patient-derived and allogeneic mDA cells into the striatum of NSG mice, patient humanized NSG mice, and allogeneic humanized NSG mice. Our findings revealed that while autologous mDA cells were rejected in allogeneic humanized mice, they were accepted in autologous humanized mice,⁶¹ indicating that allogeneic mDA cells would likely be rejected without immunosuppression when transplanted into the brain. It is noteworthy that the brain's immune privilege may require an intact blood-brain barrier (BBB), which could be compromised in PD brains¹⁰⁶ as well as by the surgical procedure. Additionally, our recent study suggests that the host immune system interacts not only with grafted cells but also with the surgical instrument, namely the needle, underscoring the critical role of the host innate immune response in the process,⁸⁴ as described below.

### Adaptive Immune Response and Immunosuppression

The adaptive immune response plays a pivotal role in the rejection of allogeneic or xenogeneic grafts.¹⁰⁷ This adaptive immunity is characterized by the precise and sensitive reactions of T and B cells to MHCs, also known as HLA, expressed by all nucleated cells. Even slight differences in the expression of HLA molecule(s) by grafted cells can trigger active elimination by the body. Therefore, significant immunosuppression is required even for intracerebral transplantation of allogeneic mDA cells. Indeed, all current and future clinical trials involving allogeneic hPSC-derived mDA cells plan to use substantial levels of immunosuppression.^{90,93,95,108} It is crucial to acknowledge that long-term immunosuppression is associated with diverse side effects, including susceptibility to infections and malignancies, along with additional costs and inconvenience. Previous studies have demonstrated a correlation between infection risk and the dose and duration of immunosuppressant,^{109,110} with risks and severity of infection increasing when used in combination with other immunosuppressants.¹¹¹ Additionally, immunosuppression with cyclosporine is known to elevate the risk of malignancies such as lymphoma or skin cancer.^{112,113} Other side effects may include loss of appetite, vomiting, nausea, increased hair growth, and tremors, although these symptoms typically subside as the body adapts to immunosuppressants. Given these various side effects, researchers are actively exploring ways to optimize the administration route, dosage and duration of immunosuppression. For example, a promising strategy involves leveraging local immunosuppression to circumvent the systemic side effects associated with standard immunosuppression.¹¹⁴ Innate immune response

Innate immunity constitutes the body's initial line of defense, encompassing physical, chemical, and cellular mechanisms to promptly counteract or eliminate foreign antigens introduced from external sources.^{107,115} Unlike adaptive immunity, which is acquired through exposure to invaders, innate immunity is inherently present at birth and relies on antigen-nonspecific defense mechanisms. The primary function of innate immunity is to directly eliminate pathogens, and most importantly, to rapidly recruit immune cells to the site of infection and inflammation.^{116,117} This is achieved through sensing pathogens and producing cytokines (e.g., TNFα, IL1β, IFNγ) and chemokines (e.g., CXCL, CCL). Additionally, innate immunity responds to physical damage resulting from external mechanical forces. A representative example is neuroinflammation induced by TBI, where initial traumatic insults trigger disruption of both macro-barriers (e.g., the skin) and micro-barriers (e.g., cell membranes), leading to secretion of diverse immune molecules.¹¹⁸

Our recent study showed that the transplantation procedure using a needle prominently triggers host innate immune responses, which was termed “needle trauma' due to its similarity to TBI (FIG. 18).⁸⁴ Primary TBI occurs immediately after the impact, damaging physical structures of the brain, including meningeal and neuronal contusion, axonal shearing, and blood vessel damage. Secondary TBI develops gradually, involving various cellular processes, such as BBB disturbance, excitotoxicity, mitochondrial dysfunction, oxidative stress, inflammation, and cell loss.¹¹⁵ Since needle trauma similarly induces these downstream pathways, further elucidation may provide promising molecular targets to mitigate its effects on the survival of grafted mDA cells, thereby potentially enhancing the outcomes of hPSC-based CRT (see below). It's worth noting that PD patients typically exhibit elevated levels of both brain inflammation and systemic inflammation compared to healthy individuals.¹¹⁹⁻¹²¹ This heightened inflammatory state in PD patients may potentially exert a more pronounced adverse impact on the outcome of CRT. Furthermore, it's essential to recognize that the systemic immune system varies significantly among individuals, including those with PD, which may lead to differences in treatment outcomes due to these immunological differences. However, it's important to acknowledge that the primary factor influencing the survival of transplanted mDANs is the inflammation triggered by brain damage resulting from the transplantation procedure itself. Consequently, further research efforts are needed to fully understand the distinct roles of intrinsic inflammatory states and surgery-induced inflammation in determining the survival of grafted mDANs.

Potential Strategies to Enhance Cell Survival for Successful hPSC-Based CRT

Although hPSC-based CRT offers the advantage for generating an unlimited number of transplantable cells, it also faces the challenge of poor survival hPSC-derived mDANs, as evidenced by previous pre-clinical studies (Table 2).^{96,122-131} Therefore, developing novel strategies to enhance the survival of mDANs during CRT is imperative. One promising approach is modifying the host immune responses to create a more favorable environment for the transplanted cells.

### Strategies Targeting Adaptive Immunity

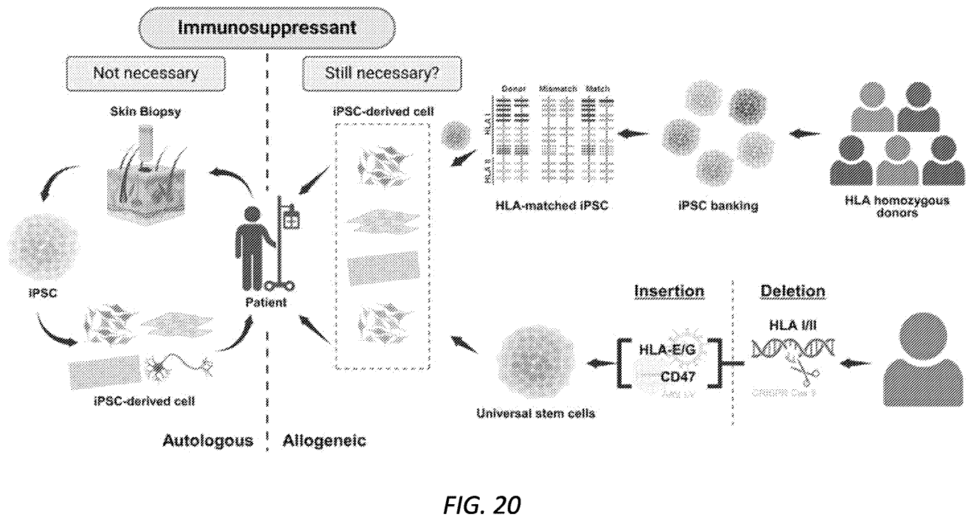

A prerequisite for the survival of grafted cells is to circumvent graft rejection. Consequently, all allogeneic transplantations require a significant level of immunosuppression, although the duration of immunosuppression required for each patient remains uncertain. Recent studies in nonhuman primates¹³²⁻¹³⁴ and human⁶¹ have demonstrated that autologous transplantations does not require immunosuppression. However, this autologous approach entails substantial time and expense. An alternative strategy to mitigate the need for immunosuppression involves using HLA-matched iPSCs. Supporting evidence for this approach comes from a recent primate study indicating that transplantation of mDA cells from HLA-matched primate iPSCs reduced host immune responses and increased the survival of mDANs.¹² Consequently, numerous groups are striving to establish HLA-matched hiPSCs as a bank from common HLA-homozygous donors, aiming to minimize graft rejection post-transplantation and reduce the time and effort compared to the autologous approach (FIG. 20).^{135,136} Professor Yamanaka's group estimated that hiPSC lines derived from approximately 140 unique HLA-homozygous donors would be sufficient to cover up to 90% of the Japanese population.¹³⁷ Due to genetic diversity, large-scale hPSC banks are currently being established in the United States to cover diverse ethnic groups such as European Americans, African Americans, Hispanics, and Asians.¹³⁸ Despite these efforts, the immune response may still occur even with HLA-matched cells due to indirect pathway caused by H-Y minor histocompatibility antigens or innate immunity resulting from natural killer (NK) cells.^{139,140}

Efforts to develop “universal donor stem cells” that evade immune rejection have garnered significant attention. 141,142 This approach primarily aims to eliminate HLA molecules in donor cells, which are pivotal for adaptive immunity and graft rejection. The initial endeavor in 2013 aimed to eliminate HLA molecules, specifically targeting HLA class I, which predominantly expresses β2-microtubulin (B2M), a structurally non-polymorphic heavy chain, across most cell types.^{143,144} However, cells lacking HLA class I, generated through genomic engineering approach, exhibited limitations as they were susceptible to lysis by NK cells via a ‘missing self’ response.¹⁴⁵ Notably, the lysis mechanism is averted when the CD94/NGK2A complex on NK cells engages with any HLA class I molecule, including HLA-E, recognized for its minimal polymorphism and expression solely in B2M knockout cells.^{145,146} Recently, through the process of technology development and optimization, Schrepfer and colleagues¹⁴⁷ devised a method to generate hypoimmunogenic donor iPSCs through three steps: (1) CRISPR/Cas9-mediated knockout of the B2M gene (a component of HLA class I) and (2) the CIITA gene (the master regulator of HLA class II), and (3) the lentiviral overexpression of the immune receptor CD47 transgene. They demonstrated that various cell types derived from these engineered donor iPSCs (e.g., endothelial cells, smooth muscle cells, and cardiomyocytes) effectively evade immune rejection in fully HLA-mismatched host animals and survive long-term without immunosuppression. Additionally, Akitsu Hotta and colleagues created pseudo-homozygous iPSC lines by disrupting HLA-A and HLA-B bi-allelically while retaining a single HLA-C allele using CRISPR-Cas9.¹⁴⁸ It is estimated that 12 HLA-C-retained iPSC lines with HLA-class II knockout could be immunologically compatible with >90% of the world's population. While these strategies hold promise for iPSC-based regenerative medicine applications, it remains uncertain whether these engineered cells would retain full functionality and hypoimmunogenicity post-transplantation into patients.¹⁴⁹ Extensive genetic manipulation (e.g., ectopic CD47 expression) may lead to unforeseen adverse effects like oncogenic transformation and compromised immune responses.^{150,151} Moreover, in instances where a cell becomes infected with a virus, prompt removal may not be feasible, prompting the need for the development of genetic integration of kill-switches. However, this strategy could potentially induce unintended adverse reactions, necessitating further research to refine and implement safety strategies.^{152,153}. Furthermore, even if these genetic alterations successfully evade immune rejection by adaptive immunity, the transplanted grafts may still be susceptible to innate immune responses by needle trauma.

### Strategies Targeting Innate Immunity

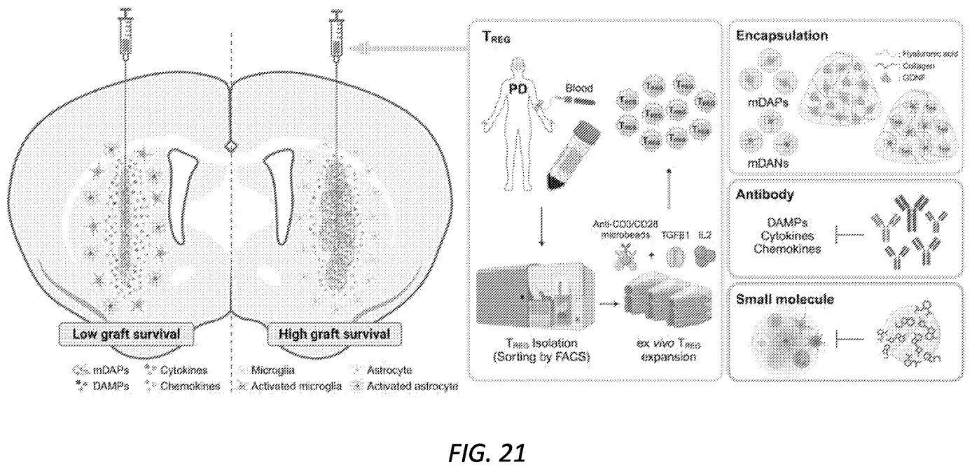

The host innate immune response triggered by TBI or needle trauma initiates inflammation within minutes after injury. This response is characterized by the secretion and upregulation of damage-associated molecular patterns (DAMPs), cytokines, chemokines, immune cell infiltration (e.g., neutrophils and myeloid cells), and subsequent activation of glial cells (astrocyte and microglia) and recruitment of leukocytes.^{107,115,154} Numerous studies have demonstrated that these molecules including DAMPs, cytokines, and chemokines, are acutely secreted after TBI within 6 hours¹⁵⁵. Consequently, various research endeavors have sought to target innate immunity for therapeutic development in TBI. These investigations have revealed that blocking those molecules using specific inhibitors resulted in prominent effects to treat TBI in animal models. 156-159 It will be intriguing to determine whether these inhibitors and/or neutralizing antibodies can produce similar effects in needle trauma and improve the survival of engrafted mDA cells. This avenue warrants future investigation (FIG. 21). Moreover, considering that needle trauma acutely triggers the secretion of various cytokines, chemokines, and DAMPs (within minutes to hours post-injury), an intriguing approach could involve delaying the grafting of cells post-needle insertion (without cells). Indeed, previous studies have shown that neuronal survival markedly increased when the injection of dopaminergic cell suspension was delayed for more than one hour following cannula insertion.^{160,161}

Regulatory T cells (T_{REG}) play a vital role in maintaining immunological tolerance and homeostasis.^{162,163} They are implicated in numerous autoimmune and inflammatory diseases and has been clinically utilized to improve survival rates in various organ transplantations through adoptive transfer after ex vivo expansion.¹⁶⁴ In TBI, T_{REG} infiltrate the damaged area following initial inflammatory cell infiltration to facilitate the repair process.^{165,166} Therefore, it was hypothesized that, autologous T_{REG} might mitigate the innate immune response triggered by needle trauma and enhance the survival of grafted mDANs. In testing the effect of T_{REG} via conventional adoptive transfer,^{164,167} approximately 1,000,000 ex vivo expanded autologous T_{REG}, were intravenously injected, leading to a modest but significant reduction in the innate immune response, as indicated by decreased infiltration of MHCII⁺ cells. However, due to the rapid onset of needle trauma-induced neuroinflammation in the brain, the adoptive transfer method may be inefficient as it requires time for the T_{REG} to infiltrate and function. Direct intra-striatal transplantation of only 2% (20,000) T_{REG} cells, on the other hand, led to robust suppression of needle trauma-induced inflammation and significantly increased the survival of grafted mDANs (Table 2), accompanied by faster and more pronounced behavioral improvement in rodent models of PD.⁸⁴ Nevertheless, the rescue of mDANs was incomplete, suggesting that further optimization of intra-striatal co-transplantation of T_{REG} is necessary. One potential approach could involve encapsulating T_{REG} and mDA cells using hydrogel systems before transplantation (FIG. 21).¹⁶⁸⁻¹⁷⁰ Additionally, considering that needle trauma may occur in CRT for other CNS and non-CNS diseases, it is of significant interest to test whether T_{REG} CO-transplantation can similarly reduce needle trauma-induced inflammation and enhance the survival of desired therapeutic cell products in general.¹⁷¹

### Strategies Targeting Long-Term Differentiation and Maturation at the Later Stage of Graft Establishment

Even if grafted mDA cells manage to evade the initial immune attacks post-transplantation and survive the early stage, they face a suboptimal environment unlike early brain development, where all necessary nutrients, oxygen, growth factors, relevant transcription factors, and developmental signals are provided or induced in a precise temporal and spatial manner. Instead, the new environment of grafted mDA cells is characterized by aged PD pathological conditions such as elevated neuroinflammation, limited/no supply of essential factors, and/or a-synucleinopathy.^{172,173} Indeed, recent single-cell RNA sequencing analyses have revealed that grafted cells often exhibit inadequate differentiation into mDANs, favoring instead the differentiation into alternative cell types such as astrocytes and vascular leptomeningeal cells.¹⁷⁴ These inadequate and heterogenous graft cell population may be non-functional and could potentially induce unwanted immunogenicity. This challenge is compounded by the lengthy process for these mDA cells to mature into mDANs with neuronal outgrowth, reinnervate to host brain, and eventually to establish functional new networks. This process can take considerable time ranging from 4-6 months in rodent brains to 1-3 years in human brains (Table 1). Therefore, it is crucial to develop and implement therapeutic strategies aimed at enhancing the survival, differentiation, and/or maturation of grafted mDA cells. Supporting this idea, pre-treatment of fVM cells with growth factors such as bFGF and GDNF increased the survival of grafted mDANs about 2-fold,^{53,54} while long-term supply of bFGF led to a 10-fold increase in the number of survived mDANs.⁵⁵ Furthermore, recent studies have demonstrated that pretreatment or viral delivery of GDNF enhanced the survival and differentiation of hPSCs (Table 2).^{123,175-177} Building upon these promising findings, a potential strategy involves facilitating the continued supply of relevant growth factor(s), which may not only improve the survival but also the differentiation of new mDANs from the graft.

TABLE 2
List of Pre-clinical Studies Using PSC-derived mDA cells

| | | | Number of | | | |
|---|---|---|---|---|---|---|
| Graft | Host | Application | grafted cells | Number of survived TH⁺ cells | | Ref. |
| hESC-mDA | NSG mouse | N/A | 150,000 | 4,500 ± 1,000 | | 94 |
| precursors | SD rat | | 250,000 | 14,500 ± 5,000 | | |
| hiPSC-NPC | NOD-SCID mouse | N/A | 1 × 10⁵ | 7,500 ± 3,500 (D 28 without Shh and FGF8) | | 131 |
| | | | | 2,000 ± 1,500 (D 28 with Shh and FGF8) | | |
| | | | | <500 (D 42 without Shh and FGF8) | | |
| | | | | <500 (D 42 with Shh and FGF8) | | |
| | Cynomolgus monkey | | 2.4 × 10⁶ | 3.07 × 10⁴ (right, d 28 spheres), | | |
| | | | (right) | 1.26 × 10⁵ (left, d 42 spheres) | | |
| | | | 2.4 × 10⁶ | | | |
| | | | (left) | | | |
| hESC-NPC | Cynomolgus monkey | N/A | 4.8 × 10⁶ | 1.3-18.6 × 10³ in each side | | 130 |
| PiPSC-mDANs | SD rat | N/A | 100,000-200,000 | 2,791-18,684 (sorted NCAM⁺/CD29^{low}) | | 129 |
| | | | | 4,835-22,666 (unsorted) | | |
| hESC-mDANs | Athymic rat | N/A | 150,000 | 986 ± 333 | | 128 |
| hiPSC-mDAPs | SD rat | Sorting: CORIN⁺ | 4 × 10⁵ | Unsorted: | CORIN⁺ sorted: | 127 |
| | | | | 3,436 ± 2,384 (D 28) | 6,747 ± 2,341 (D 28) | |
| | | | | N/A (D 42) | 1,900 ± 658 (D 42) | |
| CM-iPSC- | Cynomolgus monkey | N/A | MF25-04: 1 × 10⁷ | 13,029 (MF25-04) | | 134 |
| mDANS | | | MF27-04, MF66-02: | 8,551 (MF27-04), 7,938 (MF66-02) | | |
| | | | 4 × 10⁷ | | | |
| hESC-mDANs | SCID mouse | N/A | 2 × 10⁵ | 7,555 ± 913 (hM4Di-), 7,245 ± 517 (hM3Dq-), | | 126 |
| | | | | 6,110 ± 254 (EGFP-expressing cell) | | |
| hESC/hiPSC- | SD rat | Sorting: LRTM1⁺ | 1.3 × 10⁵ | Unsorted: | LRTM1⁺ sorted: | 125 |
| mDAPs | | | | 1,102 ± 349 | 11,702 ± 2,566 | |
| | Cynomolgus monkey | | 1 × 10⁶ | N/A | LRTM1⁺ sorted: | |
| | | | | | ~2.4 × 10⁶ | |
| hiPSC-mDAPs | Cynomolgus monkey | N/A | 4.8 × 10⁶ | Healthy: 5.4 ± 4.7 × 10⁴ | | 124 |
| | | | | PD: 7.3 ± 5.3 × 10⁴ | | |
| hESC-mDAPs | Fischer 344 rat | Encapsulation: | 89,500 (2D) | 2D, Suspension: [symbol] 1,200 | | 170 |
| | | HA-heparin- | 93,800 (3D) | 3D, HA hydrogel: [symbol] 6,400 | | |
| | | RGD hydrogel | | | | |
| hiPSC-mDAPs | Athymic rat | N/A | 100,000 | 5,621 ± 1,029 per 100,000 | | 65 |
| hESC- | Athymic rat | AAV-GDNF | 100,000 | PITX3-GFP⁺ cells: | +GDNF: | 123 |
| PITX3/LMX1A- | | | | 4,092 ± 602 | PITX3-GFP⁺ cells | |
| GFP VM mDAPs | | | | | 7,986 ± 1,375 | |
| | | | | LMX1A-GFP⁺ cells: | +GDNF: | |
| | | | | 19,810 ± 2,828 | LMX1A-GFP⁺ cells | |
| | | | | | 30,381 ± 6,513 | |
| hiPSC-mDAPs | F344-II2rgem2Kyo | Zonisamide | 5 × 10⁵ | 3.17 ± 1.64 × 10³ (vehicle) | | 122 |
| | X-SCID rat | | | 7.5 ± 1.95 × 10³ (+ZNS 30 mg/kg) | | |
| | | | | 8.16 ± 3.43 × 10³ (+ZNS 60 mg/kg) | | |
| hiPSC-mDAPs | F344 Njc1-rnu/rnu rat | N/A | 4 × 10⁵ | 2,835 ± 2,534 | | 90 |
| hESC-mDAPs | Athymic rat | N/A | 400,000 | 49,250 (male), 42,480 (female) | | 93 |
| hiPSC-mDA | F344 Njc1-rnu/rnu rat | N/A | 400,000 (fresh) | 4,601 ± 1,189 (fresh) | | 102 |
| neurospheres | | | 400,000 (cryopreserved × | 1,306 ± 480 (cryopreserved × 1) | | |
| | | | 1) | 3,988 ± 1,961 (cryopreserved × 2) | | |
| | | | 800,000 (cryopreserved × | | | |
| | | | 2) | | | |
| hiPSC-PITX3- | Athymic rat | AAV-GDNF | 100,000 | 18,910 ± 1,853 | +GDNF: | 177 |
| GFP mDAPs | | | | (Homotopic) | 28,088 ± 2,618 | |
| | | | | 20,550 ± 1,616 | (Homotopic) | |
| | | | | (Ectopic) | 24,396 ± 1,180 | |
| | | | | | (Ecotopic) | |
| hiPSC-mDAPs | NSG mouse | T_{REG} | 100,000 | +Saline: | +T_{REG}: | 84 |
| | | | | 1,825 ± 287 (2 weeks) | 4,427 ± 189 (2 weeks) | |
| | | | | 5,421 ± 534 (20 weeks) | 9,523 ± 835 (20 weeks) | |

| | | | | | |
|---|---|---|---|---|---|
| hESC-mDAPs | Athymic rat | N/A | 30,000 | [symbol] 1,900 | 95 |
| | | | 60,000 | [symbol] 3,700 | |
| | | | 120,000 | [symbol] 8,000 | |
| hESC-mDAPs | Athymic rat | N/A | 5,000 | 758.86 ± 286.00 | 96 |
| | | | 10,000 | 1,477.71 ± 529.79 | |
| | | | 25,000 | 2,820.00 ± 1,378.86 | |
| | | | 100,000 | 17,598.40 ± 5,451.54 | |

Abbreviations: hESC, human embryonic stem cell; mDA, midbrain dopaminergic; NSG, NOD SCID gamma; SD, Sprague Dawley; hiPSC, human induced pluripotent stem cell; NPC, neural progenitor cell; NOD, nonobese diabetic; SCID, severe combined immunodeficient; Shh, sonic hedgehog; FGF8, fibroblast growth factor 8; PiPSC, primate induced pluripotent stem cell; mDANs, midbrain dopaminergic neurons; NCAM, neural cell adhesion molecule; CM, Cynomolgus monkey; mDAPs, midbrain dopaminergic progenitor cells; PD, Parkinson's disease; ZNS, Zonisamide; GDNF, Glial cell line-derived neurotrophic factor; RGD, Arginylglycylaspartic acid; HA, Hyaluronic acid; T_{REG}, Regulatory T cells; Ref., Reference.

Since the hallmark pathological feature of PD is the selective loss of mDANs in the substantia nigra, CRT has been the focus of extensive study for over four decades. In particular, fVM-based CRT has provided not only proof-of-concept but also invaluable lessons. Among these, the most significant lesson is that the survival of implanted mDANs is very limited, potentially underlying the variable and often inefficient clinical outcomes. Despite significant research efforts, the molecular and cellular mechanisms underlying the acute and extensive death of mDANs post-transplantation remain only partially understood.

Recent advancements in stem cell technology offer promise for hPSC-based CRT, with various hPSC sources (e.g., autologous hiPSCs, HLA-matched hiPSCs, and allogeneic hESCs) being explored for scalable production of transplantable mDA cells in ongoing or upcoming clinical trials. However, several critical issues must be addressed for the successful implementation of hPSC-based CRT. Firstly, improving the survival of implanted mDANs is paramount, drawing from lessons learned from fVM-based CRT. Secondly, addressing the multiple phases and steps of CRT where mDANs are at risk of dying is essential. Thirdly, understanding and manipulating both host adaptive and innate immune responses are crucial, with recent evidence highlighting the role of the surgical procedure in triggering host innate immune responses. Future research efforts should focus on developing effective strategies to manipulate host immune responses to enhance the survival of implanted mDANs and improve clinical outcomes.

It is important to note that, although grafted mDANs may survive post-transplantation, they face the unfavorable PD host environment including neuroinflammation and limited supply of essential factors. These challenges make their prolonged survival and maturation very difficult, necessitating further research in this aspect. Additionally, PD patients often exhibit degeneration of other neurons beyond mDANs, such as noradrenergic and/or serotonergic neurons,¹⁷⁸⁻¹⁸¹ contributing to various non-motor deficits. Therefore, even the most successful hPSC-based CRT may not provide a ‘cure’ for PD. Instead, it is expected to be an integral component of a comprehensive treatment strategy, complementing other approaches such as novel drug treatment (e.g., anti-inflammatory and neuroprotective) and gene therapy.

### Compositions for Cell Therapy

As described above, the methods described herein include the use of compositions comprising any one or more of:
a population of T_{REG} cells;
a population of mDA cells;
an Ab that binds a pro-inflammatory cytokine (e.g., an anti-TNF-α Ab, an anti-IL-1β Ab, an anti-IFN-γ Ab), or an antibody that binds a DAMPs molecule (e.g., an anti-HMGB1 Ab, an anti-IL-1α Ab);
a composition comprising a population of T_{REG} cells and a second population of cells (e.g., mDA cells), wherein the cells can be mixed together into a single composition or administered separately; and/or
a composition comprising an Ab (e.g., an antibody that binds a pro-inflammatory cytokine or a damage-associated molecular patterns (DAMPs) molecule; optionally an anti-TNF-α Ab, an anti-IL-1β Ab, an anti-IFN-γ Ab, an anti-HMGB1 Ab, an anti-IL-1α Ab) and a population of cells (e.g., mDA cells), wherein the antibody and cells can be mixed together into a single composition or administered separately; to a subject in need thereof.

Populations of T_{REG} cells useful in the present methods are described herein and known in the art. Useful T_{REG} cells can express one or more of FoxP3, CD25, CD47, CD45, CD4, CD8, and CD127. See also US 2023/0323299, US 2023/0047159, US 2020/0330515, and references 163-167.

Populations of mDA cells useful in the present methods are described herein and known in the art. A population of mDA cells useful for the methods described herein can be derived from hESCs/hiPSCs and subsequently differentiated in vitro over a specific period using optimized procedures (FIG. 19).^{65,89-96} In some embodiments, a population of mDA cells comprises mDAPs and mDANs. In some embodiments, a population of mDA cells comprises at least about 50, 55, 60, 65, 70, 75, 80, 85, 90, or 95% mDAPs and at least about 5, 10, 15, 20, or 25% mDANs. A useful population of mDA cells can comprise cells that express one, two, or more mDAP markers (e.g., FOXA2, OTX2, LMX1A, and EN1, e.g., FOXA2 and LMX1A; optionally, TH⁺ cells that co-express FOXA2, LMX1A and NURR1); and optionally, cells that express one, two, or more mDAN markers (e.g., TH, DAT, and PITX3); and optionally, not include cells that express SOX1, PAX6, and/or KI67. See also US 2022/0243174; US 2018/0371422; US 2012/0128655; US 2013/0052268; US 2016/0002604; US 2014/0199274; and US 2009/0226401; as well as U.S. Pat. Nos. 11,898,169; 11,001,809; 9,657,273; and 9,750,768; and references 65 and 89-96.

In some embodiments, the methods described herein include administration of a composition comprising a mixture of cell types (e.g., a composition comprising both a population of Treg cells and a population of mDA cells). The methods described herein demonstrated that a significantly less number of Treg cells need to be administered with the mDA cells as opposed to when the Treg cells are administered systemically (e.g., IV). Thus, the ratio of Treg cells to mDA cells can be about 1:1, 1:2, 1:3, 1:4, 1:5, 1:6, 1:7, 1:8, 1:9, 1:10: 1:11, 1:12, 1:13, 1:14, 1:15, 1:16, 1:17, 1:18, 1:19, or 1:20.

The methods and compositions described herein can be used for and comprise other cell types, including other neuronal types. A number of neuronal differentiation protocols are known in the art; see, e.g., Salimi et al., Mol Biol Rep. 2014 March; 41(3):1713-21; Gunhnlar et al., Molecular Psychiatry 23:1336-1344 (2018); Trilck et al., Methods Mol Biol. 2016; 1353:233-59; Zhang et al., Stem Cell Res Ther. 2018 Mar. 15; 9(1):67; D'Aiuto et al., Organogenesis. 2014; 10 (4): 365-77; Marton and Ioannidis, Stem Cells Translational Medicine 2019; 8:366-374; Bell et al. Bio-protocol 9(5): e3188 (2019). DOI: 10.21769/BioProtoc.3188; Bianchi et al., Stem Cell

### Research 32:126-134 (2018).

In some embodiments, compositions including any of the cells and antibodies described herein can include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiments, the compositions are formulated to be compatible with its intended route of administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin. Useful solutions and compositions can comprise any of the solutions, buffers, and stabilizers disclosed herein; for example, a composition and/or method can include encapsulating cells (e.g., T_{REG} cells and/or mDA cells) using a hydrogel system known in the art before administration (FIG. 21).¹⁶⁸⁻¹⁷⁰

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the compositions are prepared with carriers that will protect the cells and/or antibodies against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36 (1): 49-62 (2008).

Any of the compositions described herein can be included in a container, kit, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

### REFERENCES (FOR DETAILED DESCRIPTION ONLY)

1. Fahn, S. The 200-year journey of Parkinson disease: Reflecting on the past and looking towards the future. Parkinsonism Relat Disord 46 Suppl 1, S1-S5 (2018).
2. Kalia, L. V. & Lang, A. E. Parkinson's disease. Lancet 386, 896-912 (2015).
3. Poewe, W., et al. Parkinson disease. Nat Rev Dis Primers 3, 17013 (2017).
4. Obeso, J. A., et al. Missing pieces in the Parkinson's disease puzzle. Nat Med 16, 653-661 (2010).
5. Meissner, W. G., et al. Priorities in Parkinson's disease research. Nat Rev Drug Discov 10, 377-393 (2011).
6. Barker, R. A., Barrett, J., Mason, S. L. & Bjorklund, A. Fetal dopaminergic transplantation trials and the future of neural grafting in Parkinson's disease. Lancet Neurol 12, 84-91 (2013).
7. Sonntag, K. C., et al. Pluripotent stem cell-based therapy for Parkinson's disease: Current status and future prospects. Prog Neurobiol 168, 1-20 (2018).
8. Parmar, M., Grealish, S. & Henchcliffe, C. The future of stem cell therapies for Parkinson disease. Nat Rev Neurosci 21, 103-115 (2020).
9 Barker, R. A., Drouin-Ouellet, J. & Parmar, M. Cell-based therapies for Parkinson disease-past insights and future potential. Nat Rev Neurol 11, 492-503 (2015).
10. Skidmore, S. & Barker, R. A. Challenges in the clinical advancement of cell therapies for Parkinson's disease. Nat Biomed Eng 7, 370-386 (2023).
11. Cha, Y., Park, T. Y., Leblanc, P. & Kim, K. S. Current Status and Future Perspectives on Stem Cell-Based Therapies for Parkinson's Disease. J Mov Disord 16, 22-41 (2023).
12. Lindvall, O. Clinical translation of stem cell transplantation in Parkinson's disease. J Intern Med 279, 30-40 (2016).
13. Li, J. Y. & Li, W. Postmortem Studies of Fetal Grafts in Parkinson's Disease: What Lessons Have We Learned? Front Cell Dev Biol 9, 666675 (2021).
14. Lindvall, O., et al. Grafts of fetal dopamine neurons survive and improve motor function in Parkinson's disease. Science 247, 574-577 (1990).
15. Defer, G. L., et al. Long-term outcome of unilaterally transplanted parkinsonian patients. I. Clinical approach. Brain 119 (Pt 1), 41-50 (1996).
16. Mendez, I., et al. Simultaneous intrastriatal and intranigral fetal dopaminergic grafts in patients with Parkinson disease: a pilot study. Report of three cases. J Neurosurg 96, 589-596 (2002).
17. Peschanski, M., et al. Bilateral motor improvement and alteration of L-dopa effect in two patients with Parkinson's disease following intrastriatal transplantation of foetal ventral mesencephalon. Brain 117 (Pt 3), 487-499 (1994).
18. Freed, C. R., et al. Survival of implanted fetal dopamine cells and neurologic improvement 12 to 46 months after transplantation for Parkinson's disease. N Engl J Med 327, 1549-1555 (1992).
19. Freeman, T. B., et al. Bilateral fetal nigral transplantation into the postcommissural putamen in Parkinson's disease. Ann Neurol 38, 379-388 (1995).
20. Hagell, P., et al. Sequential bilateral transplantation in Parkinson's disease: effects of the second graft. Brain 122 (Pt 6), 1121-1132 (1999).
21. Kefalopoulou, Z., et al. Long-term clinical outcome of fetal cell transplantation for Parkinson disease: two case reports. JAMA Neurol 71, 83-87 (2014).
22. Kordower, J. H., et al. Functional fetal nigral grafts in a patient with Parkinson's disease: chemoanatomic, ultrastructural, and metabolic studies. J Comp Neurol 370, 203-230 (1996).
23. Kordower, J. H., et al. Fetal nigral grafts survive and mediate clinical benefit in a patient with Parkinson's disease. Mov Disord 13, 383-393 (1998).
24. Lindvall, O., et al. Human fetal dopamine neurons grafted into the striatum in two patients with severe Parkinson's disease. A detailed account of methodology and a 6-month follow-up. Arch Neurol 46, 615-631 (1989).
25. Lindvall, O., et al. Evidence for long-term survival and function of dopaminergic grafts in progressive Parkinson's disease. Ann Neurol 35, 172-180 (1994).
26. Lindvall, O., et al. Transplantation of fetal dopamine neurons in Parkinson's disease: one-year clinical and neurophysiological observations in two patients with putaminal implants. Ann Neurol 31, 155-165 (1992).
27. Mendez, I., et al. Cell type analysis of functional fetal dopamine cell suspension transplants in the striatum and substantia nigra of patients with Parkinson's disease. Brain 128, 1498-1510 (2005).
28. Piccini, P., et al. Dopamine release from nigral transplants visualized in vivo in a Parkinson's patient. Nat Neurosci 2, 1137-1140 (1999).
29. Spencer, D. D., et al. Unilateral transplantation of human fetal mesencephalic tissue into the caudate nucleus of patients with Parkinson's disease. N Engl J Med 327, 1541-1548 (1992).
30. Wenning, G. K., et al. Short- and long-term survival and function of unilateral intrastriatal dopaminergic grafts in Parkinson's disease. Ann Neurol 42, 95-107 (1997).
31. Widner, H., et al. Bilateral fetal mesencephalic grafting in two patients with parkinsonism induced by 1-methyl-4-phenyl-1,2,3,6-tetrahydropyridine (MPTP). N Engl J Med 327, 1556-1563 (1992).
32. Li, W., et al. Extensive graft-derived dopaminergic innervation is maintained 24 years after transplantation in the degenerating parkinsonian brain. Proc Natl Acad Sci USA 113, 6544-6549 (2016).
33. Kordower, J. H., Chu, Y., Hauser, R. A., Freeman, T. B. & Olanow, C. W. Lewy body-like pathology in long-term embryonic nigral transplants in Parkinson's disease. Nat Med 14, 504-506 (2008).
34. Li, J. Y., et al. Lewy bodies in grafted neurons in subjects with Parkinson's disease suggest host-to-graft disease propagation. Nat Med 14, 501-503 (2008).
35. Mendez, I., et al. Dopamine neurons implanted into people with Parkinson's disease survive without pathology for 14 years. Nat Med 14, 507-509 (2008).
36. Greene, P. E., et al. Persistent dyskinesias in patients with fetal tissue transplantation for Parkinson disease. NPJ Parkinsons Dis 7, 38 (2021).
37. Freed, C. R., et al. Transplantation of embryonic dopamine neurons for severe Parkinson's disease. N Engl J Med 344, 710-719 (2001).
38. Olanow, C. W., et al. A double-blind controlled trial of bilateral fetal nigral transplantation in Parkinson's disease. Ann Neurol 54, 403-414 (2003).
39. Thompson, L. & Bjorklund, A. Survival, differentiation, and connectivity of ventral mesencephalic dopamine neurons following transplantation. Prog Brain Res 200, 61-95 (2012).
40. Ramachandran, A. C., Bartlett, L. E. & Mendez, I. M. A multiple target neural transplantation strategy for Parkinson's disease. Rev Neurosci 13, 243-256 (2002).
41. Castilho, R. F., Hansson, O. & Brundin, P. Improving the survival of grafted embryonic dopamine neurons in rodent models of Parkinson's disease. Prog Brain Res 127, 203-231 (2000).
42. Bjorklund, A. & Kordower, J. H. Cell therapy for Parkinson's disease: what next? Mov Disord 28, 110-115 (2013).
43. Brundin, P., et al. Improving the survival of grafted dopaminergic neurons: a review over current approaches. Cell Transplant 9, 179-195 (2000).
44. Brundin, P., et al. Human fetal dopamine neurons grafted in a rat model of Parkinson's disease: immunological aspects, spontaneous and drug-induced behaviour, and dopamine release. Exp Brain Res 70, 192-208 (1988).
45. Frodl, E. M., Duan, W. M., Sauer, H., Kupsch, A. & Brundin, P. Human embryonic dopamine neurons xenografted to the rat: effects of cryopreservation and varying regional source of donor cells on transplant survival, morphology and function. Brain Res 647, 286-298 (1994).
46. Barker, R. A., Dunnett, S. B., Faissner, A. & Fawcett, J. W. The time course of loss of dopaminergic neurons and the gliotic reaction surrounding grafts of embryonic mesencephalon to the striatum. Exp Neurol 141, 79-93 (1996).
47. Duan, W. M., Widner, H. & Brundin, P. Temporal pattern of host responses against intrastriatal grafts of syngeneic, allogeneic or xenogeneic embryonic neuronal tissue in rats. Exp Brain Res 104, 227-242 (1995).
48. Emgard, M., Karlsson, J., Hansson, O. & Brundin, P. Patterns of cell death and dopaminergic neuron survival in intrastriatal nigral grafts. Exp Neurol 160, 279-288 (1999).
49. Wenker, S. D. & Pitossi, F. J. Cell therapy for Parkinson's disease is coming of age: current challenges and future prospects with a focus on immunomodulation. Gene Ther 27, 6-14 (2020).
50. Zawada, W. M., et al. Growth factors improve immediate survival of embryonic dopamine neurons after transplantation into rats. Brain Res 786, 96-103 (1998).
51. Schierle, G. S., et al. Caspase inhibition reduces apoptosis and increases survival of nigral transplants. Nat Med 5, 97-100 (1999).
52. Mahalik, T. J., Hahn, W. E., Clayton, G. H. & Owens, G. P. Programmed cell death in developing grafts of fetal substantia nigra. Exp Neurol 129, 27-36 (1994).
53. Zeng, B. Y., Jenner, P. & Marsden, C. D. Altered motor function and graft survival produced by basic fibroblast growth factor in rats with 6-OHDA lesions and fetal ventral mesencephalic grafts are associated with glial proliferation. Exp Neurol 139, 214-226 (1996).
54. Mayer, E., Fawcett, J. W. & Dunnett, S. B. Basic fibroblast growth factor promotes the survival of embryonic ventral mesencephalic dopaminergic neurons--II. Effects on nigral transplants in vivo. Neuroscience 56, 389-398 (1993).
55. Takayama, H., et al. Basic fibroblast growth factor increases dopaminergic graft survival and function in a rat model of Parkinson's disease. Nat Med 1, 53-58 (1995).
56. Schierle, G. S. & Brundin, P. Excitotoxicity plays a role in the death of tyrosine hydroxylase-immunopositive nigral neurons cultured in serum-free medium. Exp Neurol 157, 338-348 (1999).
57. Nakao, N., Frodl, E. M., Duan, W. M., Widner, H. & Brundin, P. Lazaroids improve the survival of grafted rat embryonic dopamine neurons. Proc Natl Acad Sci USA 91, 12408-12412 (1994).
58. Kaminski Schierle, G. S., Hansson, O. & Brundin, P. Flunarizine improves the survival of grafted dopaminergic neurons. Neuroscience 94, 17-20 (1999).
59. Schweitzer, J. S., Song, B. & Kim, K. S. A step closer to autologous cell therapy for Parkinson's disease. Cell Stem Cell 28, 595-597 (2021).
60. Merkle, F. T., et al. Human pluripotent stem cells recurrently acquire and expand dominant negative P53 mutations. Nature 545, 229-233 (2017).
61. Schweitzer, J. S., et al. Personalized iPSC-Derived Dopamine Progenitor Cells for Parkinson's Disease. N Engl J Med 382, 1926-1932 (2020).
62. Lee, A. S., Tang, C., Rao, M. S., Weissman, I. L. & Wu, J. C. Tumorigenicity as a clinical hurdle for pluripotent stem cell therapies. Nat Med 19, 998-1004 (2013).
63. Yamanaka, S. Pluripotent Stem Cell-Based Cell Therapy-Promise and Challenges. Cell Stem Cell 27, 523-531 (2020).
64. Lee, M. O., et al. Inhibition of pluripotent stem cell-derived teratoma formation by small molecules. Proc Natl Acad Sci USA 110, E3281-3290 (2013).
65. Song, B., et al. Human autologous iPSC-derived dopaminergic progenitors restore motor function in Parkinson's disease models. J Clin Invest 130, 904-920 (2020).
66. Ostrom, Q. T., et al. The epidemiology of glioma in adults: a “state of the science” review. Neuro Oncol 16, 896-913 (2014).
67. Lund, R. J., Narva, E. & Lahesmaa, R. Genetic and epigenetic stability of human pluripotent stem cells. Nat Rev Genet 13, 732-744 (2012).
68. Yoshihara, M., Hayashizaki, Y. & Murakawa, Y. Genomic Instability of iPSCs: Challenges Towards Their Clinical Applications. Stem Cell Rev 13, 7-16 (2017).
69. Abyzov, A., et al. Somatic copy number mosaicism in human skin revealed by induced pluripotent stem cells. Nature 492, 438-442 (2012).
70. Cheng, L., et al. Low incidence of DNA sequence variation in human induced pluripotent stem cells generated by nonintegrating plasmid expression. Cell Stem Cell 10, 337-344 (2012).
71. Gore, A., et al. Somatic coding mutations in human induced pluripotent stem cells. Nature 471, 63-67 (2011).
72. Howden, S. E., et al. Genetic correction and analysis of induced pluripotent stem cells from a patient with gyrate atrophy. Proc Natl Acad Sci USA 108, 6537-6542 (2011).
73. Quinlan, A. R., et al. Genome sequencing of mouse induced pluripotent stem cells reveals retroelement stability and infrequent DNA rearrangement during reprogramming. Cell Stem Cell 9, 366-373 (2011).
74. Ji, J., et al. Elevated Coding Mutation Rate During the Reprogramming of Human Somatic Cells into Induced Pluripotent Stem Cells. Stem Cells 30, 435-440 (2012).
75. Sugiura, M., et al. Induced Pluripotent Stem Cell Generation-Associated Point Mutations Arise during the Initial Stages of the Conversion of These Cells. Stem Cell Reports 2, 52-63 (2014).
76. Yoshihara, M., et al. Hotspots of De Novo Point Mutations in Induced Pluripotent Stem Cells. Cell Rep 21, 308-315 (2017).
77. Liu, P., et al. Passage number is a major contributor to genomic structural variations in mouse iPSCs. Stem Cells 32, 2657-2667 (2014).
78. Lezmi, E., Jung, J. & Benvenisty, N. High prevalence of acquired cancer-related mutations in 146 human pluripotent stem cell lines and their differentiated derivatives. Nat Biotechnol (2024).
79. Trounson, A. Potential Pitfall of Pluripotent Stem Cells. N Engl J Med 377, 490-491 (2017).
80. Wang, L., et al. Immunogenicity and functional evaluation of iPSC-derived organs for transplantation. Cell Discov 1, 15015 (2015).
81. Guha, P., Morgan, J. W., Mostoslavsky, G., Rodrigues, N. P. & Boyd, A. S. Lack of immune response to differentiated cells derived from syngeneic induced pluripotent stem cells. Cell Stem Cell 12, 407-412 (2013).
82. Araki, R., et al. Negligible immunogenicity of terminally differentiated cells derived from induced pluripotent or embryonic stem cells. Nature 494, 100-104 (2013).
83. Zhao, T., et al. Humanized Mice Reveal Differential Immunogenicity of Cells Derived from Autologous Induced Pluripotent Stem Cells. Cell Stem Cell 17, 353-359 (2015).
84. Park, T. Y., et al. Co-transplantation of autologous T (reg) cells in a cell therapy for Parkinson's disease. Nature 619, 606-615 (2023).
85. Ozaki, M., et al. Evaluation of the immunogenicity of human iPS cell-derived neural stem/progenitor cells in vitro. Stem Cell Res 19, 128-138 (2017).
86. Kimura, T., Yamashita, A., Ozono, K. & Tsumaki, N. Limited Immunogenicity of Human Induced Pluripotent Stem Cell-Derived Cartilages. Tissue Eng Part A 22, 1367-1375 (2016).
87. Alam, A., et al. Cellular infiltration in traumatic brain injury. J Neuroinflammation 17, 328 (2020).
88. Karve, I. P., Taylor, J. M. & Crack, P. J. The contribution of astrocytes and microglia to traumatic brain injury. Br J Pharmacol 173, 692-702 (2016).
89. Kirkeby, A., et al. Predictive Markers Guide Differentiation to Improve Graft Outcome in Clinical Translation of hESC-Based Therapy for Parkinson's Disease. Cell Stem Cell 20, 135-148 (2017).
90. Doi, D., et al. Pre-clinical study of induced pluripotent stem cell-derived dopaminergic progenitor cells for Parkinson's disease. Nat Commun 11, 3369 (2020).
91. Gantner, C. W., Cota-Coronado, A., Thompson, L. H. & Parish, C. L. An Optimized Protocol for the Generation of Midbrain Dopamine Neurons under Defined Conditions. STAR Protoc 1, 100065 (2020).
92. Xiong, M., et al. Human Stem Cell-Derived Neurons Repair Circuits and Restore Neural Function. Cell Stem Cell 28, 112-126 el16 (2021).
93. Piao, J., et al. Preclinical Efficacy and Safety of a Human Embryonic Stem Cell-Derived Midbrain Dopamine Progenitor Product, MSK-DA01. Cell Stem Cell 28, 217-229 e217 (2021).
94. Kriks, S., et al. Dopamine neurons derived from human ES cells efficiently engraft in animal models of Parkinson's disease. Nature 480, 547-551 (2011).
95. Kirkeby, A., et al. Preclinical quality, safety, and efficacy of a human embryonic stem cell-derived product for the treatment of Parkinson's disease, STEM-PD. Cell Stem Cell 30, 1299-1314 e1299 (2023).
96. Park, S., et al. Preclinical and dose-ranging assessment of hESC-derived dopaminergic progenitors for a clinical trial on Parkinson's disease. Cell Stem Cell 31, 25-38 e28 (2024).
97. Kim, J., et al. Spotting-based differentiation of functional dopaminergic progenitors from human pluripotent stem cells. Nat Protoc 17, 890-909 (2022).
98. Nikkhah, G., Eberhard, J., Olsson, M. & Bjorklund, A. Preservation of fetal ventral mesencephalic cells by cool storage: in-vitro viability and TH-positive neuron survival after microtransplantation to the striatum. Brain Res 687, 22-34 (1995).
99. Barker, R. A., Fricker, R. A., Abrous, D. N., Fawcett, J. & Dunnett, S. B. A comparative study of preparation techniques for improving the viability of nigral grafts using vital stains, in vitro cultures, and in vivo grafts. Cell Transplant 4, 173-200 (1995).
100. Watts, C., Caldwell, M. A. & Dunnett, S. B. The development of intracerebral cell-suspension implants is influenced by the grafting medium. Cell Transplant 7, 573-583 (1998).
101. Wakeman, D. R., et al. Cryopreservation Maintains Functionality of Human iPSC Dopamine Neurons and Rescues Parkinsonian Phenotypes In Vivo. Stem Cell Reports 9, 149-161 (2017).
102. Hiramatsu, S., et al. Cryopreservation of Induced Pluripotent Stem Cell-Derived Dopaminergic Neurospheres for Clinical Application. J Parkinsons Dis 12, 871-884 (2022).
103. Niclis, J. C., et al. Efficiently Specified Ventral Midbrain Dopamine Neurons from Human Pluripotent Stem Cells Under Xeno-Free Conditions Restore Motor Deficits in Parkinsonian Rodents. Stem Cells Transl Med 6, 937-948 (2017).
104. Forrester, J. V., McMenamin, P. G. & Dando, S. J. CNS infection and immune privilege. Nat Rev Neurosci 19, 655-671 (2018).
105. Zhao, T., Zhang, Z. N., Rong, Z. & Xu, Y. Immunogenicity of induced pluripotent stem cells. Nature 474, 212-215 (2011).
106. Lee, H. & Pienaar, I. S. Disruption of the blood-brain barrier in Parkinson's disease: curse or route to a cure? Front Biosci (Landmark Ed) 19, 272-280 (2014).
107. Waisman, A., Liblau, R. S. & Becher, B. Innate and adaptive immune responses in the CNS. Lancet Neurol 14, 945-955 (2015).
108. Park, S. C., et al. Does age at onset of first major depressive episode indicate the subtype of major depressive disorder?: the clinical research center for depression study. Yonsei Med J 55, 1712-1720 (2014).
109. Cutolo, M., et al. Use of glucocorticoids and risk of infections. Autoimmun Rev 8, 153-155 (2008).
110. Stuck, A. E., Minder, C. E. & Frey, F. J. Risk of infectious complications in patients taking glucocorticosteroids. Rev Infect Dis 11, 954-963 (1989).
111. Aberra, F. N. & Lichtenstein, G. R. Methods to avoid infections in patients with inflammatory bowel disease. Inflamm Bowel Dis 11, 685-695 (2005).
112. Price, M. L., Tidman, M. J., Ogg, C. S. & MacDonald, D. M. Skin cancer and cyclosporine therapy. N Engl J Med 313, 1420 (1985).
113. Muellenhoff, M. W. & Koo, J. Y. Cyclosporine and skin cancer: an international dermatologic perspective over 25 years of experience. A comprehensive review and pursuit to define safe use of cyclosporine in dermatology. J Dermatolog Treat 23, 290-304 (2012).
114. Alemdar, A. Y., Sadi, D., McAlister, V. & Mendez, I. Intracerebral co-transplantation of liposomal tacrolimus improves xenograft survival and reduces graft rejection in the hemiparkinsonian rat. Neuroscience 146, 213-224 (2007).
115. Huber-Lang, M., Lambris, J. D. & Ward, P. A. Innate immune responses to trauma. Nat Immunol 19, 327-341 (2018).
116. Duan, T., Du, Y., Xing, C., Wang, H. Y. & Wang, R. F. Toll-Like Receptor Signaling and Its Role in Cell-Mediated Immunity. Front Immunol 13, 812774 (2022).
117. Akira, S., Uematsu, S. & Takeuchi, O. Pathogen recognition and innate immunity. Cell 124, 783-801 (2006).
118. Zheng, R. Z., et al. Neuroinflammation Following Traumatic Brain Injury: Take It Seriously or Not. Front Immunol 13, 855701 (2022).
119. Collins, L. M., Toulouse, A., Connor, T. J. & Nolan, Y. M. Contributions of central and systemic inflammation to the pathophysiology of Parkinson's disease. Neuropharmacology 62, 2154-2168 (2012).
120. Yacoubian, T. A., et al. Brain and Systemic Inflammation in De Novo Parkinson's Disease. Mov Disord 38, 743-754 (2023).
121. Tansey, M. G., et al. Inflammation and immune dysfunction in Parkinson disease. Nat Rev Immunol 22, 657-673 (2022).
122. Miyawaki, Y., Samata, B., Kikuchi, T., Nishimura, K. & Takahashi, J. Zonisamide promotes survival of human-induced pluripotent stem cell-derived dopaminergic neurons in the striatum of female rats. J Neurosci Res 98, 1575-1587 (2020).
123. Gantner, C. W., et al. Viral Delivery of GDNF Promotes Functional Integration of Human Stem Cell Grafts in Parkinson's Disease. Cell Stem Cell 26, 511-526 e515 (2020).
124. Kikuchi, T., et al. Human iPS cell-derived dopaminergic neurons function in a primate Parkinson's disease model. Nature 548, 592-596 (2017).
125. Samata, B., et al. Purification of functional human ES and iPSC-derived midbrain dopaminergic progenitors using LRTM1. Nat Commun 7, 13097 (2016).
126. Chen, Y., et al. Chemical Control of Grafted Human PSC-Derived Neurons in a Mouse Model of Parkinson's Disease. Cell Stem Cell 18, 817-826 (2016).
127. Doi, D., et al. Isolation of human induced pluripotent stem cell-derived dopaminergic progenitors by cell sorting for successful transplantation. Stem Cell Reports 2, 337-350 (2014).
128. Grealish, S., et al. Human ESC-derived dopamine neurons show similar preclinical efficacy and potency to fetal neurons when grafted in a rat model of Parkinson's disease. Cell Stem Cell 15, 653-665 (2014).
129. Sundberg, M., et al. Improved cell therapy protocols for Parkinson's disease based on differentiation efficiency and safety of hESC-, hiPSC-, and non-human primate iPSC-derived dopaminergic neurons. Stem Cells 31, 1548-1562 (2013).
130. Doi, D., et al. Prolonged maturation culture favors a reduction in the tumorigenicity and the dopaminergic function of human ESC-derived neural cells in a primate model of Parkinson's disease. Stem Cells 30, 935-945 (2012).
131. Kikuchi, T., et al. Survival of human induced pluripotent stem cell-derived midbrain dopaminergic neurons in the brain of a primate model of Parkinson's disease. J Parkinsons Dis 1, 395-412 (2011).
132. Emborg, M. E., et al. Induced pluripotent stem cell-derived neural cells survive and mature in the nonhuman primate brain. Cell Rep 3, 646-650 (2013).
133. Tao, Y., et al. Autologous transplant therapy alleviates motor and depressive behaviors in parkinsonian monkeys. Nat Med 27, 632-639 (2021).
134. Hallett, P. J., et al. Successful function of autologous iPSC-derived dopamine neurons following transplantation in a non-human primate model of Parkinson's disease. Cell Stem Cell 16, 269-274 (2015).
135. de Rham, C. & Villard, J. Potential and limitation of HLA-based banking of human pluripotent stem cells for cell therapy. J Immunol Res 2014, 518135 (2014).
136. Ford, E., et al. Human Pluripotent Stem Cells-Based Therapies for Neurodegenerative Diseases: Current Status and Challenges. Cells 9 (2020).
137. Akabayashi, A., Nakazawa, E. & Jecker, N. S. Endangerment of the iPSC stock project in Japan: on the ethics of public funding policies. J Med Ethics 44, 700-702 (2018).
138. Gourraud, P. A., Gilson, L., Girard, M. & Peschanski, M. The role of human leukocyte antigen matching in the development of multiethnic “haplobank” of induced pluripotent stem cell lines. Stem Cells 30, 180-186 (2012).
139. Morizane, A., et al. MHC matching improves engraftment of iPSC-derived neurons in non-human primates. Nat Commun 8, 385 (2017).
140. Mizukami, Y., et al. MHC-matched induced pluripotent stem cells can attenuate cellular and humoral immune responses but are still susceptible to innate immunity in pigs. PLOS One 9, e98319 (2014).
141. Lanza, R., Russell, D. W. & Nagy, A. Engineering universal cells that evade immune detection. Nat Rev Immunol 19, 723-733 (2019).
142. Simpson, A., Hewitt, A. W. & Fairfax, K. A. Universal cell donor lines: A review of the current research. Stem Cell Reports 18, 2038-2046 (2023).
143. Riolobos, L., et al. HLA engineering of human pluripotent stem cells. Mol Ther 21, 1232-1241 (2013).
144. Lu, P., et al. Generating hypoimmunogenic human embryonic stem cells by the disruption of beta 2-microglobulin. Stem Cell Rev Rep 9, 806-813 (2013).
145. Bix, M., et al. Rejection of class I MHC-deficient haemopoietic cells by irradiated MHC-matched mice. Nature 349, 329-331 (1991).
146. Gornalusse, G. G., et al. HLA-E-expressing pluripotent stem cells escape allogeneic responses and lysis by NK cells. Nat Biotechnol 35, 765-772 (2017).
147. Deuse, T., et al. Hypoimmunogenic derivatives of induced pluripotent stem cells evade immune rejection in fully immunocompetent allogeneic recipients. Nat Biotechnol 37, 252-258 (2019).
148. Xu, H., et al. Targeted Disruption of HLA Genes via CRISPR-Cas9 Generates iPSCs with Enhanced Immune Compatibility. Cell Stem Cell 24, 566-578 e567 (2019).
149. Shani, T. & Hanna, J. H. Universally non-immunogenic iPSCs. Nat Biomed Eng 3, 337-338 (2019).
150. Trounson, A., Boyd, N. R. & Boyd, R. L. Toward a Universal Solution: Editing Compatibility into Pluripotent Stem Cells. Cell Stem Cell 24, 508-510 (2019).
151. Kosicki, M., Tomberg, K. & Bradley, A. Repair of double-strand breaks induced by CRISPR-Cas9 leads to large deletions and complex rearrangements. Nat Biotechnol 36, 765-771 (2018).
152. Liang, Q., et al. Linking a cell-division gene and a suicide gene to define and improve cell therapy safety. Nature 563, 701-704 (2018).
153. Harding, J., Vintersten-Nagy, K. & Nagy, A. Universal Stem Cells: Making the Unsafe Safe. Cell Stem Cell 27, 198-199 (2020).
154. Morganti-Kossmann, M. C., Rancan, M., Otto, V. I., Stahel, P. F. & Kossmann, T. Role of cerebral inflammation after traumatic brain injury: a revisited concept. Shock 16, 165-177 (2001).
155. Helmy, A., Carpenter, K. L., Menon, D. K., Pickard, J. D. & Hutchinson, P. J. The cytokine response to human traumatic brain injury: temporal profiles and evidence for cerebral parenchymal production. J Cereb Blood Flow Metab 31, 658-670 (2011).
156. Yang, S. H., Gangidine, M., Pritts, T. A., Goodman, M. D. & Lentsch, A. B. Interleukin 6 mediates neuroinflammation and motor coordination deficits after mild traumatic brain injury and brief hypoxia in mice. Shock 40, 471-475 (2013).
157. Clausen, F., et al. Neutralization of interleukin-1 beta modifies the inflammatory response and improves histological and cognitive outcome following traumatic brain injury in mice. Eur J Neurosci 30, 385-396 (2009).
158. Kabadi, S. V., et al. S100B inhibition reduces behavioral and pathologic changes in experimental traumatic brain injury. J Cereb Blood Flow Metab 35, 2010-2020 (2015).
159. Chio, C. C., et al. Therapeutic evaluation of etanercept in a model of traumatic brain injury. J Neurochem 115, 921-929 (2010).
160. Zietlow, R., Sinclair, S. R., Schwiening, C. J., Dunnett, S. B. & Fawcettt, J. W. The release of excitatory amino acids, dopamine, and potassium following transplantation of embryonic mesencephalic dopaminergic grafts to the rat striatum, and their effects on dopaminergic neuronal survival in vitro. Cell Transplant 11, 637-652 (2002).
161. Sinclair, S. R., Fawcett, J. W. & Dunnett, S. B. Delayed implantation of nigral grafts improves survival of dopamine neurones and rate of functional recovery. Neuroreport 10, 1263-1267 (1999).
162. Vignali, D. A., Collison, L. W. & Workman, C. J. How regulatory T cells work. Nat Rev Immunol 8, 523-532 (2008).
163. Wood, K. J. & Sakaguchi, S. Regulatory T cells in transplantation tolerance. Nat Rev Immunol 3, 199-210 (2003).
164. Bluestone, J. A., Mckenzie, B. S., Beilke, J. & Ramsdell, F. Opportunities for Treg cell therapy for the treatment of human disease. Front Immunol 14, 1166135 (2023).
165. Shi, L., et al. Treg cell-derived osteopontin promotes microglia-mediated white matter repair after ischemic stroke. Immunity 54, 1527-1542 e1528 (2021).
166. Ma, X., et al. Regulatory T cells protect against brain damage by alleviating inflammatory response in neuromyelitis optica spectrum disorder. J Neuroinflammation 18, 201 (2021).
167. Duggleby, R., Danby, R. D., Madrigal, J. A. & Saudemont, A. Clinical Grade Regulatory CD4(+) T Cells (Tregs): Moving Toward Cellular-Based Immunomodulatory Therapies. Front Immunol 9, 252 (2018).
168. Penna, V., et al. Extracellular Matrix Biomimetic Hydrogels, Encapsulated with Stromal Cell-Derived Factor 1, Improve the Composition of Foetal Tissue Grafts in a Rodent Model of Parkinson's Disease. Int J Mol Sci 23 (2022).
169. Moriarty, N., Cabre, S., Alamilla, V., Pandit, A. & Dowd, E. Encapsulation of young donor age dopaminergic grafts in a GDNF-loaded collagen hydrogel further increases their survival, reinnervation, and functional efficacy after intrastriatal transplantation in hemi-Parkinsonian rats. Eur J Neurosci 49, 487-496 (2019).
170. Adil, M. M., et al. Engineered hydrogels increase the post-transplantation survival of encapsulated hESC-derived midbrain dopaminergic neurons. Biomaterials 136, 1-11 (2017).
171. Tang, Q. Regulatory T cells aid stem-cell therapy for Parkinson's disease. Nature 619, 470-472 (2023).
172. Hoban, D. B., et al. Impact of alpha-synuclein pathology on transplanted hESC-derived dopaminergic neurons in a humanized alpha-synuclein rat model of PD. Proc Natl Acad Sci USA 117, 15209-15220 (2020).
173. Nolan, Y. M., Sullivan, A. M. & Toulouse, A. Parkinson's disease in the nuclear age of neuroinflammation. Trends Mol Med 19, 187-196 (2013).
174. Tiklova, K., et al. Single cell transcriptomics identifies stem cell-derived graft composition in a model of Parkinson's disease. Nat Commun 11, 2434 (2020).
175. Di Santo, S., Meyer, M., Ducray, A. D., Andereggen, L. & Widmer, H. R. A Combination of NT-4/5 and GDNF Is Favorable for Cultured Human Nigral Neural Progenitor Cells. Cell Transplant 27, 648-653 (2018).
176. Studer, L. & Tabar, V. Parkinson's disease grafts benefit from well-timed growth factor. Nature 582, 39-40 (2020).
177. Moriarty, N., et al. A combined cell and gene therapy approach for homotopic reconstruction of midbrain dopamine pathways using human pluripotent stem cells. Cell Stem Cell 29, 434-448 e435 (2022).
178. Nahimi, A., Kinnerup, M. B., Sommerauer, M., Gjedde, A. & Borghammer, P. Molecular Imaging of the Noradrenergic System in Idiopathic Parkinson's Disease. Int Rev Neurobiol 141, 251-274 (2018).
179. Espay, A. J., LeWitt, P. A. & Kaufmann, H. Norepinephrine deficiency in Parkinson's disease: the case for noradrenergic enhancement. Mov Disord 29, 1710-1719 (2014).
180. Samanci, B., Tan, S., Michielse, S., Kuijf, M. L. & Temel, Y. The habenula in Parkinson's disease: Anatomy, function, and implications for mood disorders-A narrative review. J Chem Neuroanat 136, 102392 (2024).
181. Vegas-Suarez, S., et al. Dysfunction of serotonergic neurons in Parkinson's disease and dyskinesia. Int Rev Neurobiol 146, 259-279 (2019).

### Examples

The invention is further described in the following examples, which do not limit the scope of the invention described in the claims.

### Methods Used in the Examples and Experiments

### Animals

Fischer 344 (8-10 weeks of age, male) rats, Sprague Dawley (8-10 weeks of age, male) rats and NOD SCID gamma (NOD.Cg-Prkdc^{SCID} Il2rg^{tm1Wjl}/SzJ, 8-10 weeks of age, male) mice were purchased from Charles River or The Jackson Laboratory and housed under individually-ventilated micro isolation cages under specific pathogen-free (SPF) condition at the Mailman Research Center Animal Care Facility of McLean Hospital. All animals were maintained on a 12-h: 12-h light/dark cycle with access to food and water ad libitum. Animal studies were performed in compliance with National Institutes of Health guidelines and were approved by McLean Hospital's Institutional Animal Care and Use Committee (2015N000001 and 2015N000002).

C4-mDAPs Differentiation from PD Patient hiPSC (C4-hiPSC)

We previously described the efficient generation of mDA progenitor cells (mDAPs) through differentiation of human induced pluripotent stem cells (hiPSC) using our novel “spotting”-based method^{21,22}. Briefly, C4-hiPSC were plated on Matrigel coated 12 spots (spot on a grid of 6 cross point of lines) and were kept in those plates from D1 to D14 of differentiation. To pattern the differentiating cells to the floor plate and mDAPs, 10 μM SB431542 (D1-8), 200 nM LDN193189 (D1-12), 100 ng/ml SHH (D2-10), 100 ng/ml FGF8 (D2-10), 2 μM Purmorphamine (D2-10) and 1 μM CHIR99021 (D4-12) were added to the culture medium. On day 9, to eliminate remaining hiPSC, cells were treated with 40 μM quercetin for 16 hours. On D15 of differentiation, cells were dissociated to single cells and re-plated on Poly-L-ornithine/Fibronectin/Laminin-coated dishes at approximately 2.5 million/dish in induction medium supplemented with 20 ng/ml BDNF, 20 ng/ml GDNF, 500 μM dbcAMP, 200 μM ascorbic acid, 10 ng/ml TGF-β3, 10 μM DAPT (D12-15). mDAP cells (D15 cells) were fed every 2 days and maintained without passaging until day 28. More detailed C4-hiPSC reprogramming and C4-mDAPs differentiation have been described previously^{16,21,22}. B1-mDAPs or H9-mDAPs were generated using the same in vitro differentiation procedure from an independent hiPSC line derived from a separate sporadic PD patient (B1) and the H9-hESC line, respectively. All cells were tested for mycoplasma contamination every four weeks using Venor GeM Mycoplasma Detection Kit (MP0025, Sigma-Aldrich). All experiments were performed using cells that were shown to be mycoplasma-negative.

In Vitro Differentiation of hiPSC and hESC into GABAergic Neurons and Progenitors

We differentiated hiPSC and hESC into GABAergic progenitors, as described in Liu, et al⁵⁴. Briefly, C4-hiPSC, B1-hiPSC or H9-hESC were trypsinized and grown as floating spheres in low adherent flasks from DO to D14 of differentiation. To pattern differentiating cells to GABAergic progenitors, 2 mM L-glutamine (D0-14), 10 μM B-mercaptoethanol (D0-14), 100 nM LDN193189 (D0-14), 10 μM SB431542 (DO-7), 5 μM IWP2 (DO-7), 0.1 μM SAG (DO-21) were added to the KSR medium. From day 14, cells were grown in N2AA medium with 100 ng/ml FGF8 (D14-21), 10 ng/ml GDNF and 10 ng/ml BDNF. After 3 weeks of differentiation, cIN sphere were trypsinized, plated on poly-L-ornithine/Fibronectin-coated plates in B27 GB medium, and harvested and transplanted on the appropriate day.

### Surgical Procedure

Animals were anesthetized with isoflurane using a SomnoSuite Anesthesia System (Kent Scientific Corporation, Torrington, CT, USA) and stereotaxic surgery was performed using a stereotaxic frame (David KOPF Instruments, Tujunga, CA, USA) equipped with a Micro4 micro syringe pump controller (World Precision Instruments, Sarasota, FL, USA). Massive unilateral lesions of the nigrostriatal pathway (>95% loss of striatal DA) were produced by stereotaxic injection of 6-hydroxydopamine (6-OHDA) into the medial forebrain bundle (rat) or substantia nigra pars compacta (mouse). Rats were injected with 10 mg/kg of desipramine to protect noradrenergic projections 15 minutes prior to anesthesia. Two microliters of 6-OHDA (7.5 mg/ml in 0.2% ascorbic acid and 0.9% saline) were injected using a 2.5 μl Hamilton syringe (Hamilton Company, Reno, NV). The coordinates were calculated with reference to bregma: antero-posterior (AP), −4.0; medio-lateral (ML), −1.3; and dorso-ventral (DV), −7.0. For intra-striatal transplantation of C4-mDAPs with or without T_{REG} (or only TP media; Control), one deposit of 2 μl was placed at the following coordinates: AP, +0.8; ML, −3.0; and DV, −5.5. C4-mDAPs (100,000) were transplanted through a 10 μl Hamilton syringe fitted with a blunt 26G, 0.75-inch needle at a speed of 0.4 μl/min. After injection, the needle was held in the brain for 5 min, then the needle was slowly withdrawn over a period of 5 min, the incised skin was sealed with Autoclip® Surgical Suture (Fine science tools, Foster City, CA) and animals were monitored on a warm pad until recovery. The CsA-injected group of rats received daily injection of cyclosporine A (10 mg/kg, i.p.) starting from one day before transplantation with C4-mDAPs.

Mice were injected with 25 mg/kg of desipramine to protect noradrenergic projections 15 minutes prior to anesthesia. One microliter of 6-OHDA (3.0 mg/ml in 0.2% ascorbic acid and 0.9% saline) was injected using a 2.5 μl Hamilton syringe (Hamilton Company, Reno, NV). The coordinates were calculated with reference to bregma: antero-posterior (AP), −2.9; medio-lateral (ML), −1.1; and dorso-ventral (DV), −4.5. For intra-striatal transplantation of C4-mDAPs with or without T_{REG}, one deposit of 2 μl was placed at the following coordinates: AP, +0.5; ML, −1.8; and DV, −3.5. C4-mDAPs (100,000) were transplanted through a 10 μl Hamilton syringe fitted with a blunt 26G, 0.75-inch needle at a speed of 0.4 μl/min. After injection, the needle was held in the brain for 5 min, then the needle was slowly withdrawn over a period of 5 min, the incised skin was sealed with Autoclip® Surgical Suture (Fine science tools, Foster City, CA) and animals were monitored on a warm pad until recovery.

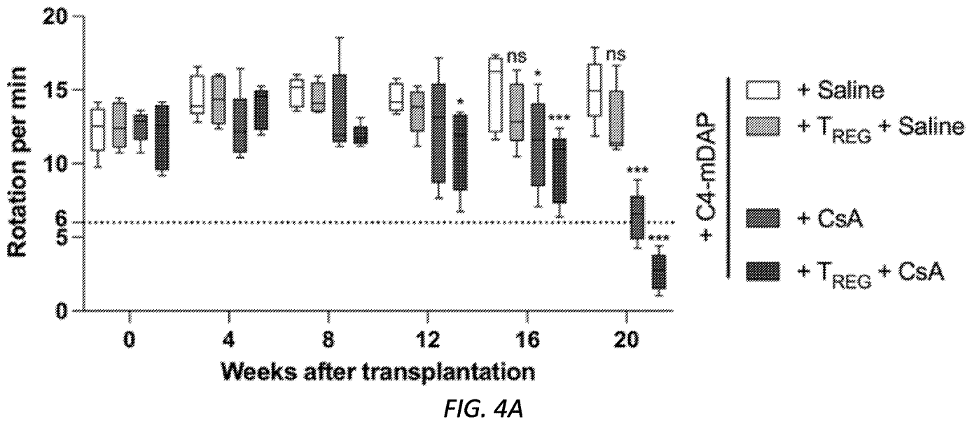

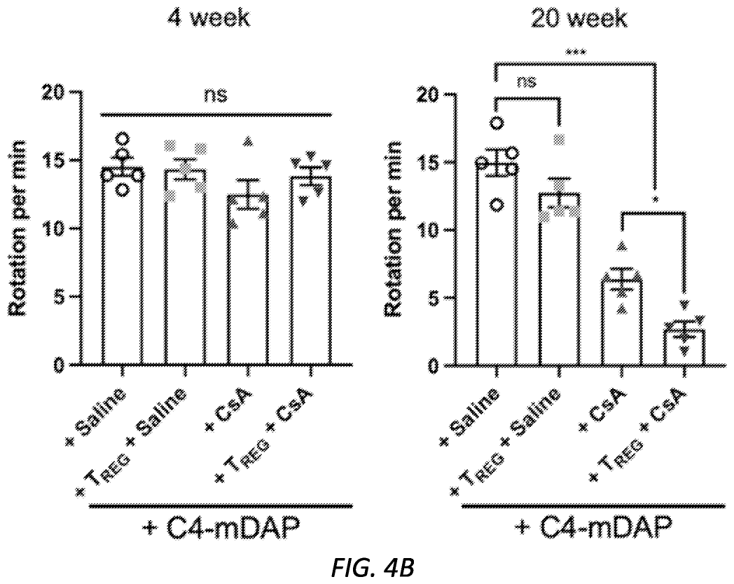

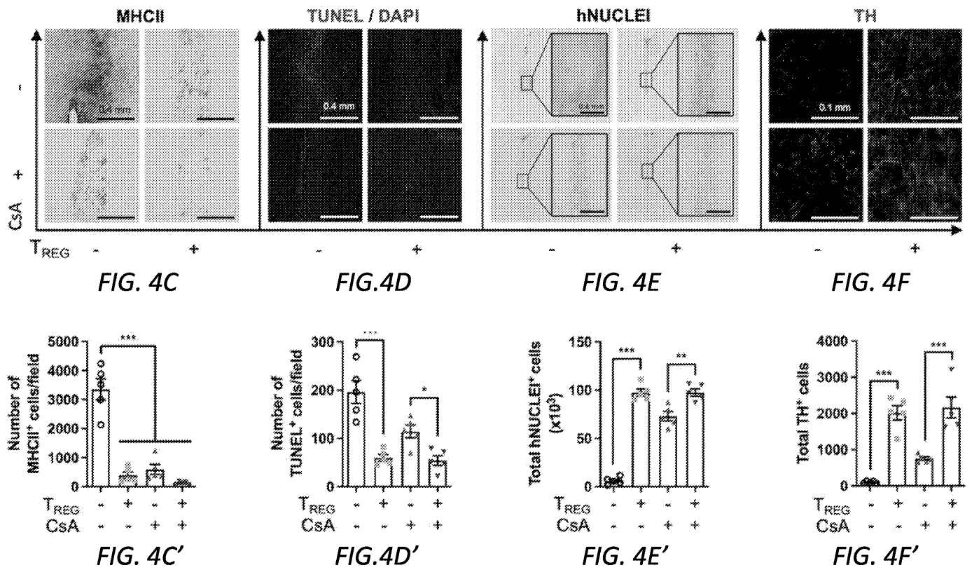

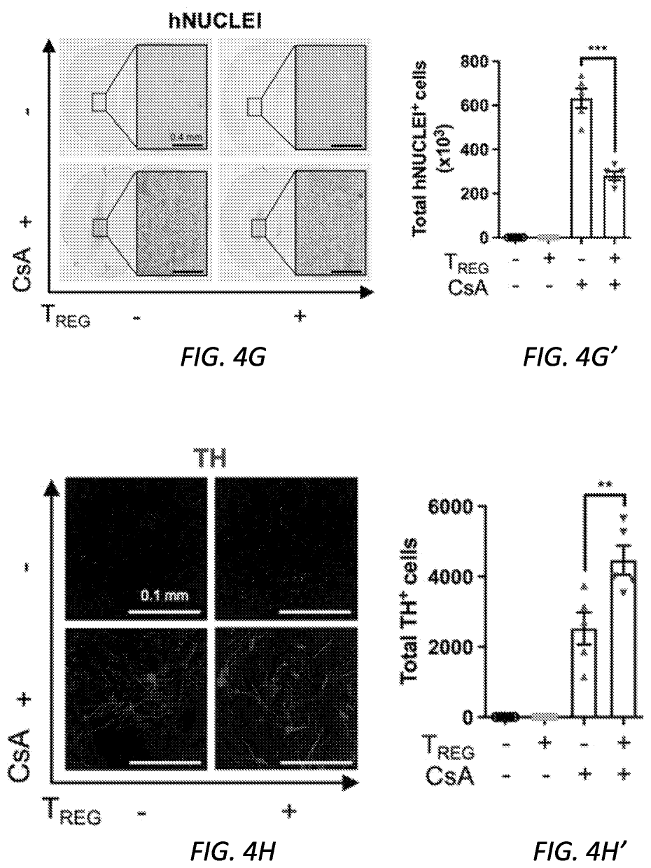

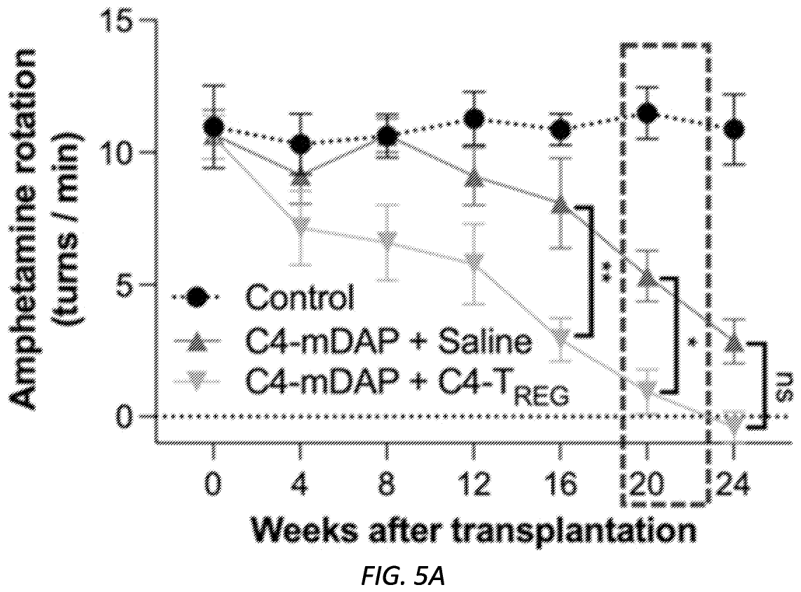

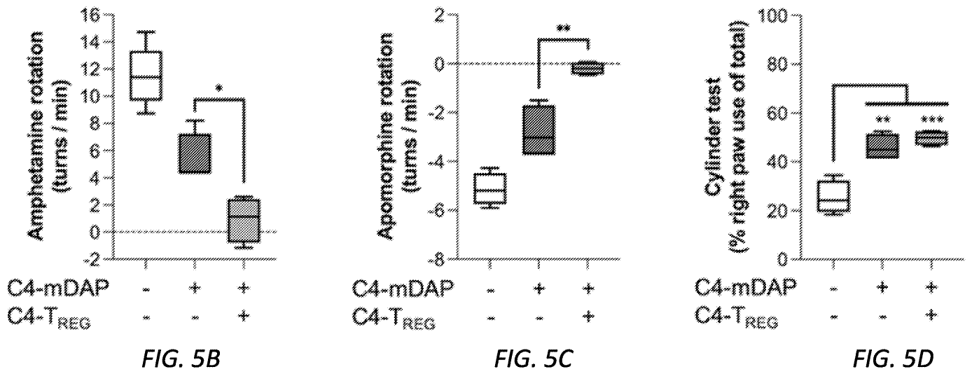

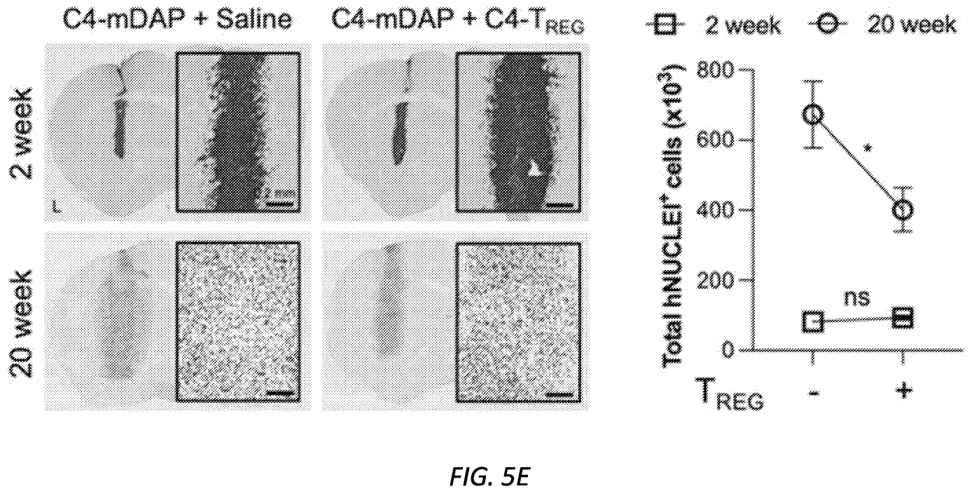

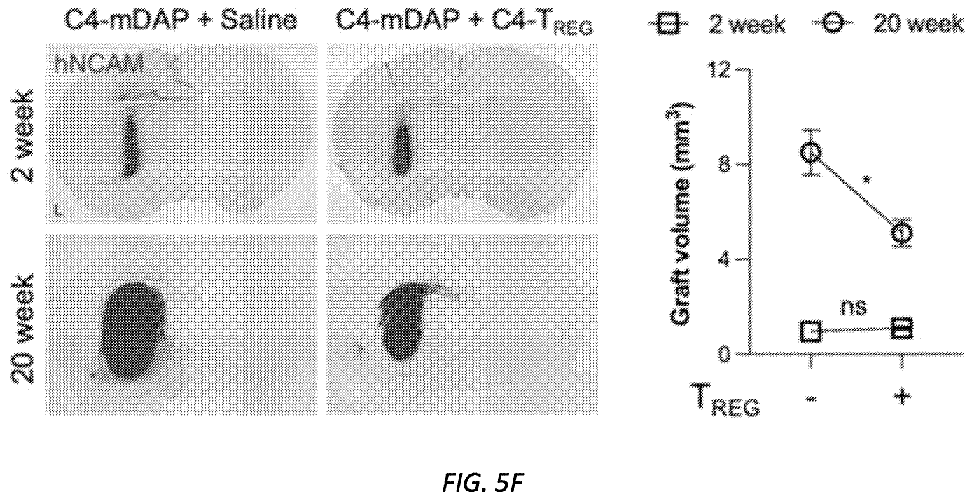

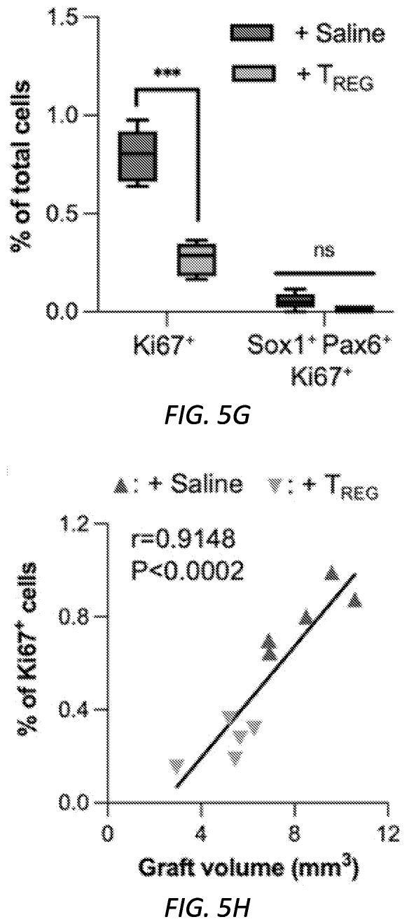

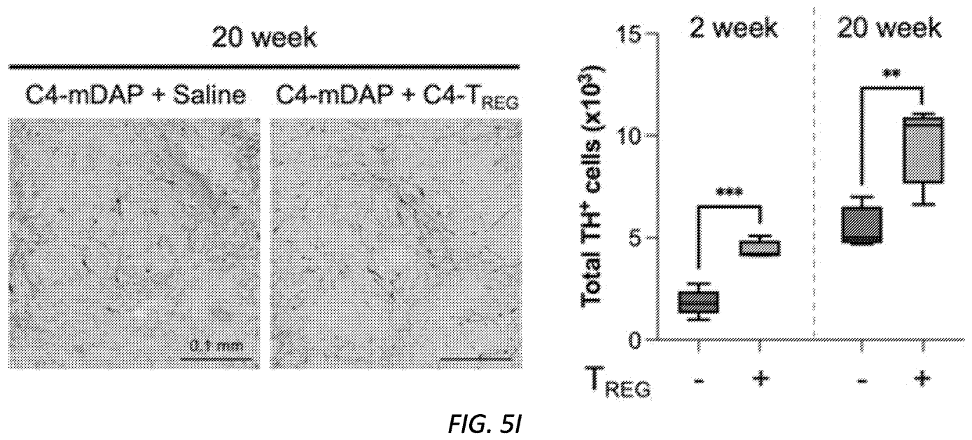

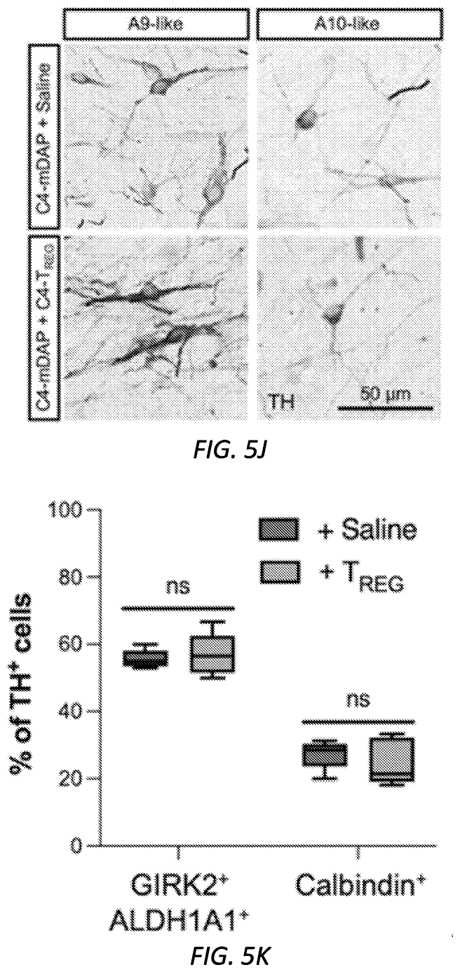

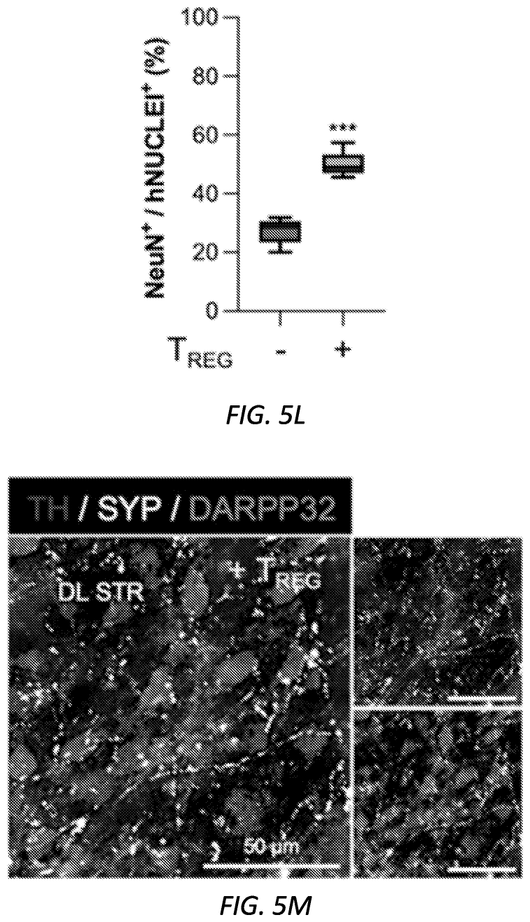

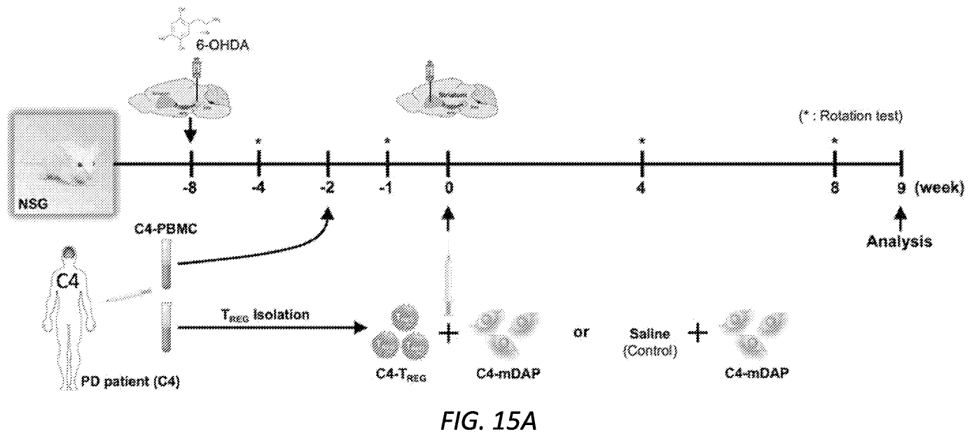

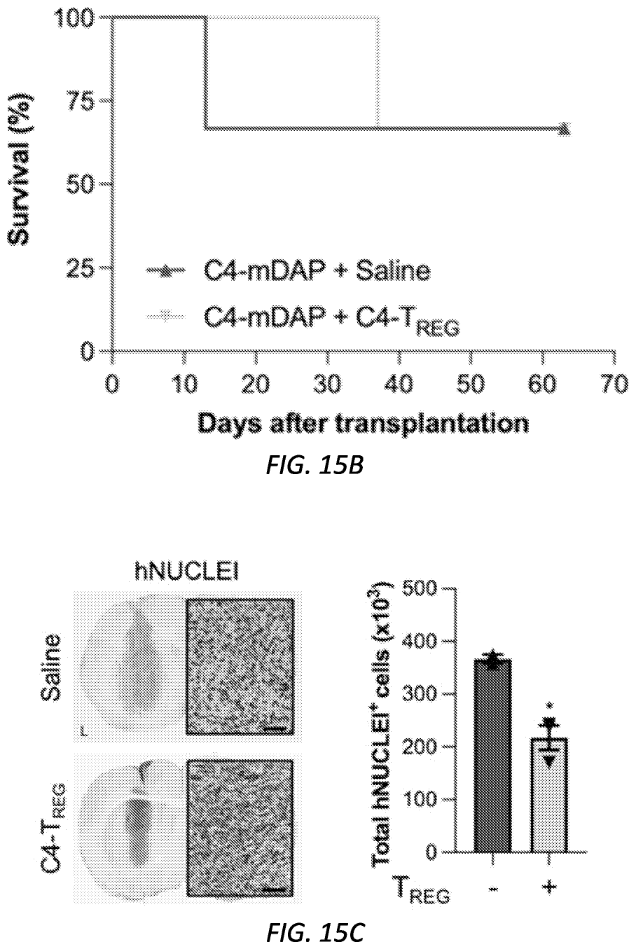

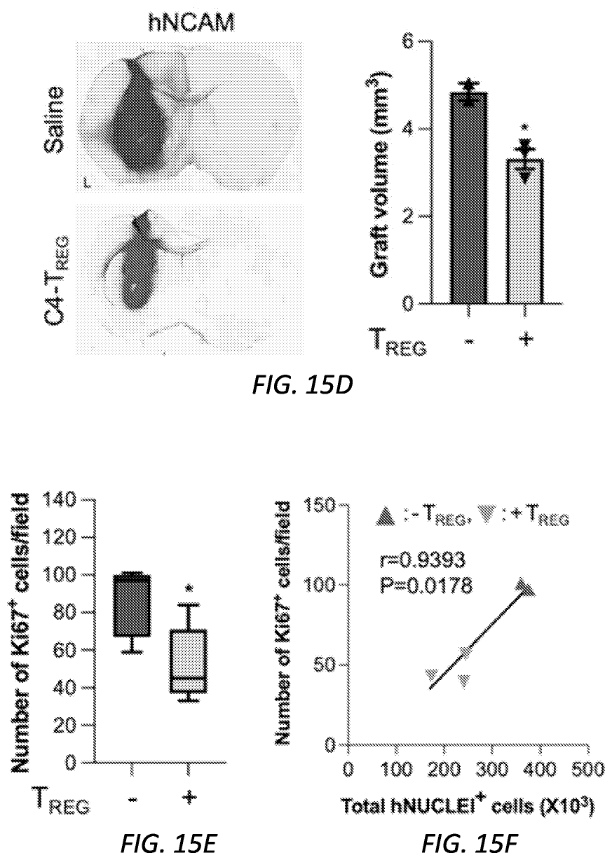

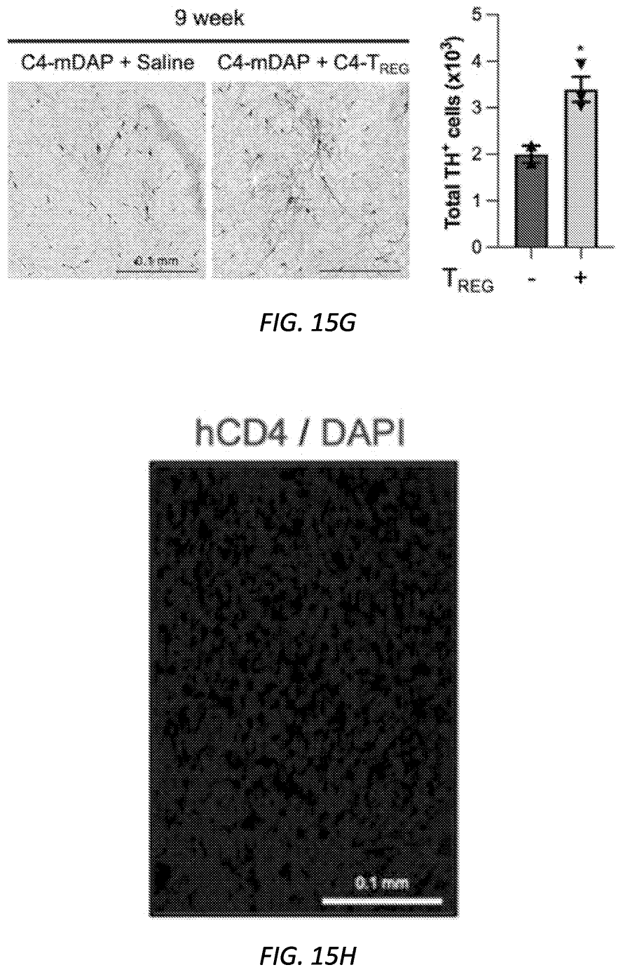

All groups in each animal experiments used the same batch of C4-mDAPs (FIGS. 4a-4h; Batch 1, FIGS. 5a-5m; Batch 2, FIGS. 15a-15h; Batch 3). In all animal experiments, the same experimental set was transplanted on the same day. For example, in the Fischer 344 animal experiment (FIGS. 4a-4h), 4 groups analyzed at 2w were transplanted on the same day (DO), and the same 4 groups analyzed at 20w were transplanted the next day (D+1).

### Humanized Mice

The PD mouse model was induced by injecting 6-OHDA in the substantia nigra of NSG mice (described above). PBMC were isolated from patient's (C4) blood and injected peritoneally into PD mice to generate C4-hu mice reconstituted with the immune system of patient C4. More detailed information has been previously described¹⁶.

### Rotation Test

D-Amphetamine, an indirect presynaptic DA agonist, was administrated intraperitoneally (4 mg/kg/rat, 5 mg/kg/mouse) to induce rotational behavior in animals successfully lesioned with 6-OHDA. Apomorphine, a direct-acting postsynaptic DA agonist, was administrated subcutaneously (1 mg/kg/mouse) to induce rotational behavior. The rotational bias was recorded for 30 or 90 min using an automated system (SD Instruments, San Diego, CA). The number of full body rotations toward the side of lesion is counted as a positive value, and only those animals showing 6 or more net ipsilateral rotations per minute are considered successfully lesioned.

### Cylinder Test

To measure the motor asymmetry of forelimb use, mice were placed in a glass cylinder (20 cm in diameter) and a minimum of 30 paw touches to the walls were recorded for 10 min. The 10 min video recordings of the mouse behavior were analyzed by two investigators who were blinded to experimental groups by counting the number of wall touches with the left, the right and both paws together. Results were calculated as an average of touches using the right-side paw and percentage of average of total touches.

### T_{REG} Isolation

Rat T_{REG} were isolated from the blood of Fischer 344 rats. Human T_{REG} were isolated from patient's blood (C4) or PBMC purchased from STEMCELL™ (K2). Blood samples were collected in anticoagulant collection tubes from the jugular vein and PBMC were isolated using SepMate™ tube by density gradient centrifugation. Rat PBMC were stained with APC-conjugated anti-CD4 (550057, BD) and PE-conjugated anti-CD25 (554866, BD) antibodies, according to manufacturer's instructions, and isolated using a FACS sorter. Human PBMC were stained with Easy Sep™ Human CD4⁺CD127^{low}CD25⁺ Regulatory T cells isolation kit (18063, STEMCELL Technologies) according to manufacturer's instructions, and isolated using an EasySep™ magnet. Isolated T_{REG} and T_{naïve} were maintained in complete medium (RPMI1640 containing 10% heat-inactivated FBS, 100 μg/ml penicillin/streptomycin). Syngeneic T_{REG}: Fischer 344 (donor, A)/Fischer 344 (recipient, A′); Autologous T_{REG}: Fischer 344 (donor/recipient, same rat).

### Ex Vivo T_{REG} Expansion

Isolated rat CD4⁺ CD25⁺ T cells were cultured in 96-well round-bottom plates (2×10⁴ cells/well) and activated with plate-bound anti-rat CD3/CD28 (5 μg/ml, each) and rIL-2 (50 U/ml). Cells were maintained at a concentration of 1×10⁶ cells/ml by changing or adding culture medium every 2~3 days. Cells were reactivated at day 10 using the initial activation conditions and maintain with IL-2. Expanded cells were harvested at various time points.

T_{REG} suppression assay

Rat conventional CD4+ T cells (T_{conv}) were labeled with CellTrace™ CFSE (Life Technologies, Invitrogen™) and stimulated with plate-bound anti-CD3 and soluble CD28 antibodies. Rat T_{REG} were added at a ratio of 1:1 or 1:2 to the 96-well round-bottom plates seeded with CFSE-labeled T_{conv} cells. The cells were kept in complete medium for 48 h. CFSE dilution of T_{conv} cells was analyzed by flow cytometry.

FLOW cytometry

To facilitate intracellular staining for Foxp3 (320012, Biolegend), CD4+CD25⁺ T cells were fixed and permeabilized using Foxp3 fixation/permeabilization buffers according to the manufacturer's instructions. C4-mDAPs were dissociated with Accutase and filtered through a 70 μm cell strainer to make single cell suspensions, fixed with 4% formaldehyde, and stained with PE-conjugated anti-HLA-ABC (560168, BD) and FITC-conjugated anti-HLA-DR (555811, BD) or FITC-conjugated anti-CD80 (560926, BD), PE-conjugated anti-CD86 (560957, BD), PE-conjugated anti-CD40 (568581, BD), PE-conjugated anti-PD-L1 (568080, BD), PE-conjugated anti-PDL2 (558066, BD) and PE-conjugated anti-CD47 (568090, BD) antibodies. Fluorochrome matched isotype controls were used and subtracted during analysis.

### TUNEL Assay

Cell death was determined with Abcam's TUNEL assay kit (ab66110, Abcam) according to the manufacturer's instructions. Frozen-brain sections were fixed in 4% formaldehyde for 15 min and washed twice with PBS. Sections were incubated in 20 μg/ml proteinase K for 5-15 min at RT, washed twice, then incubated with DNA labeling solution containing TdT reaction enzyme and Br-dUTP for 1 h in a dark humidified 37° C. incubator. Labeled sections were washed with rinse buffer, incubated with antibody solution containing anti-BrdU-Red antibody and counterstained with DAPI. The main field surrounding the area damaged by needle trauma was selected and TUNEL⁻ cells were counted using 5 rats per group and 2-3 sections per rat. C4-mDAPs cultured in vitro were treated with or without 100 ng/ml IFN-γ, 20 ng/ml TNF-α, or 10 ng/ml IL-1β for 7 days and cell death was confirmed by the same method. The number of TUNEL⁺ cells was calculated in 3-5 randomly selected areas in the field and the same experiment was independently repeated 3 times. Cell counting was conducted by two investigators who were blinded to experimental group assignment.

### Apoptosis Assay

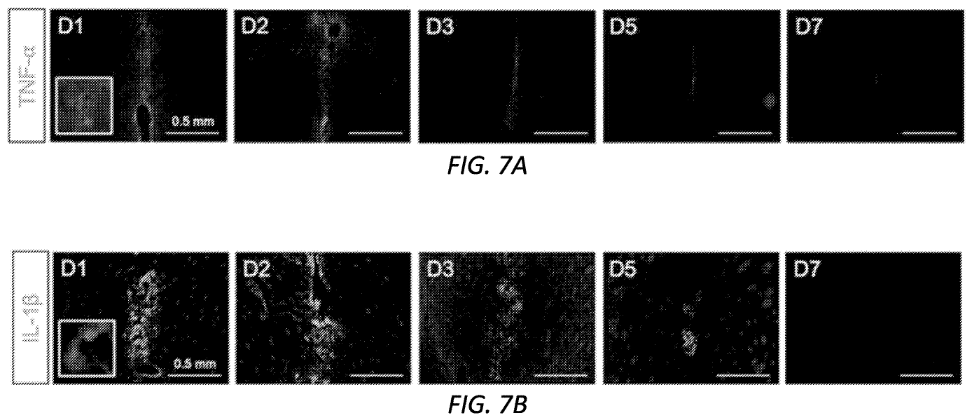

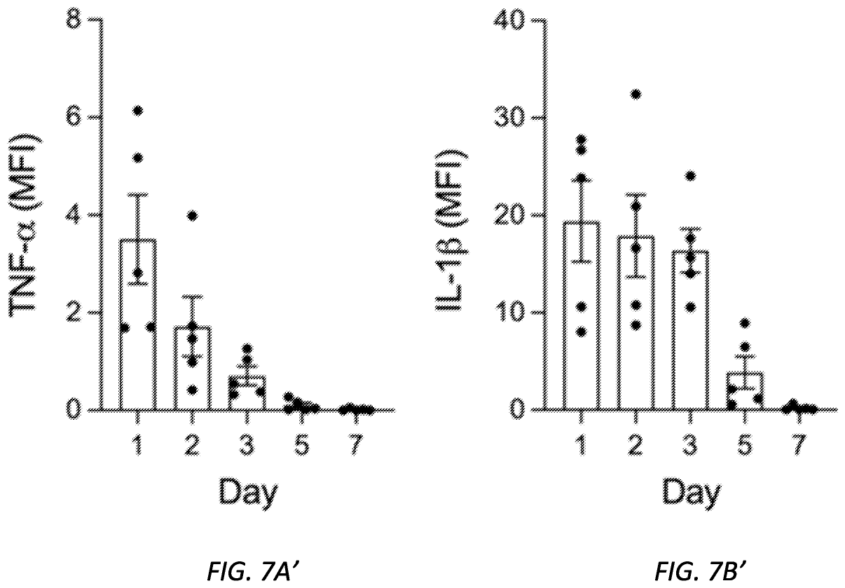

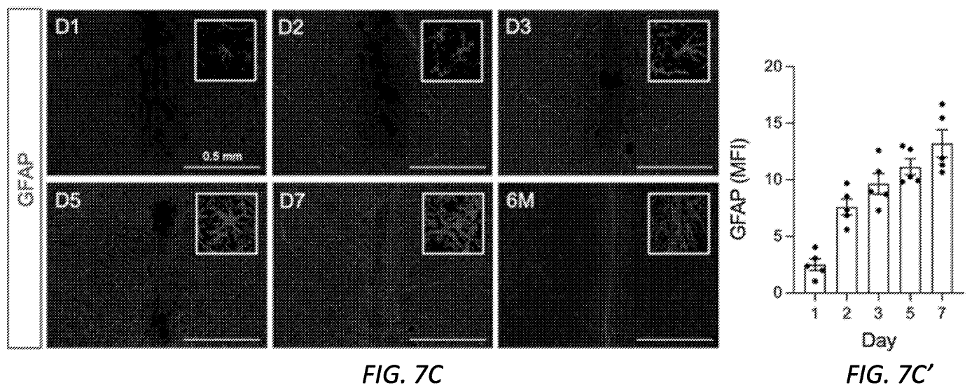

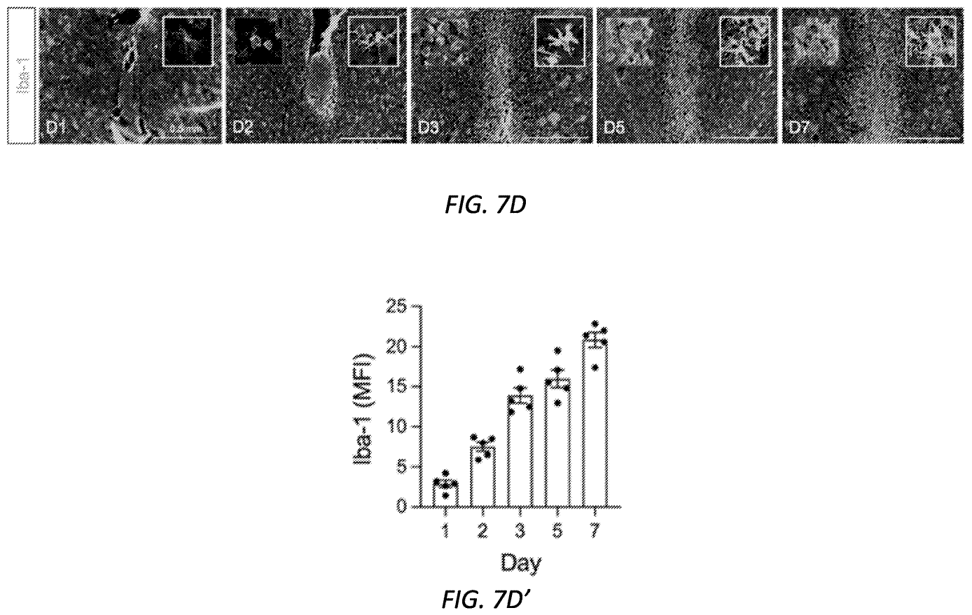

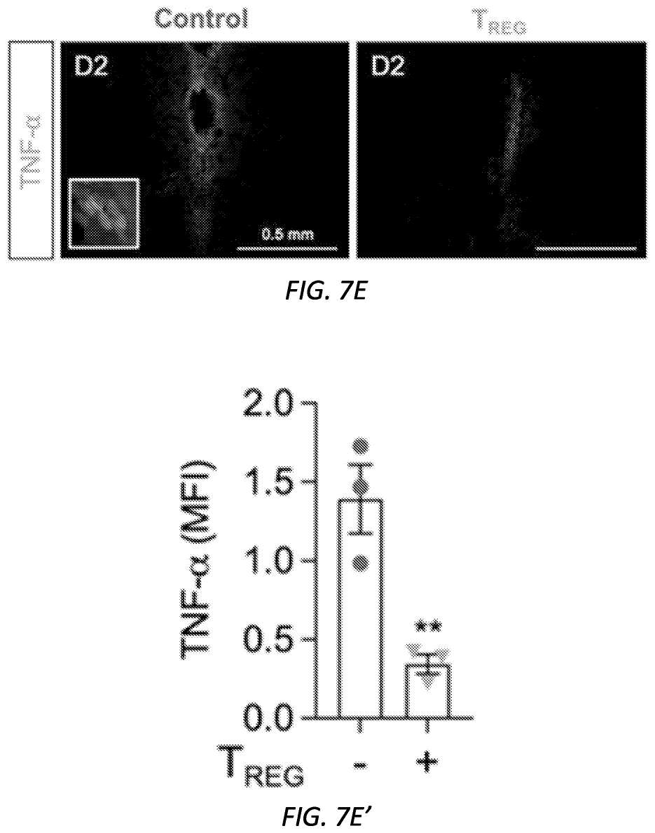

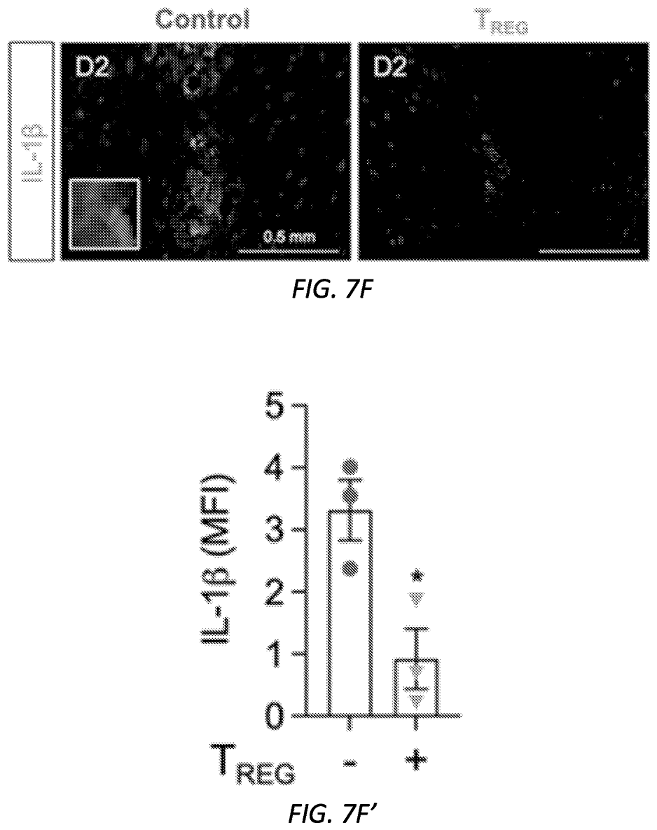

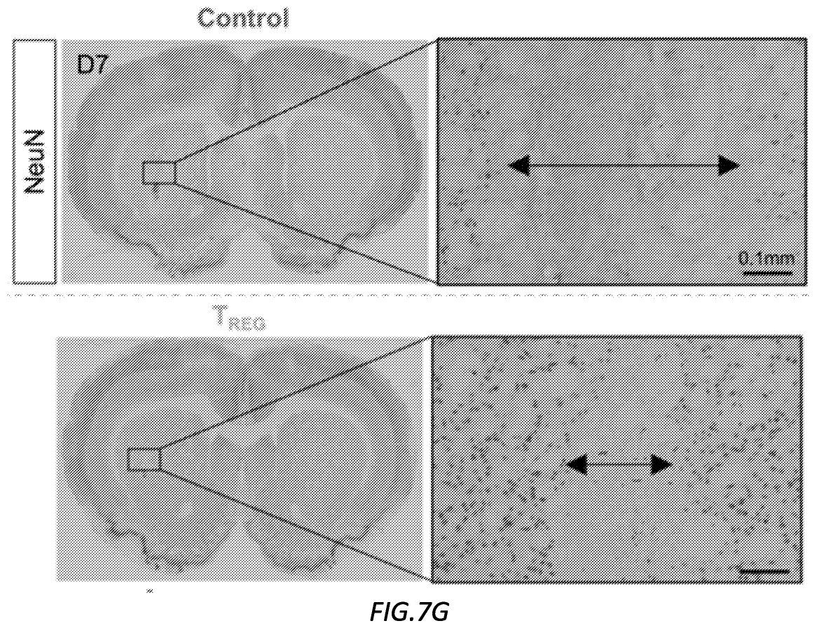

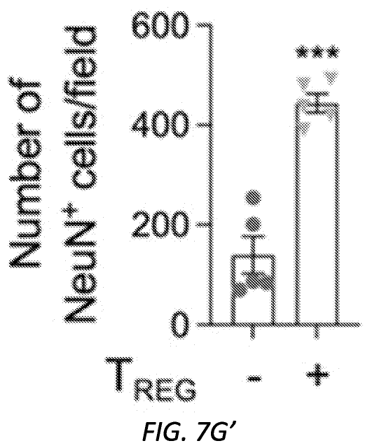

The proportion of apoptotic cells was determined with 7-aminoactinomycin D (7-AAD) and a FITC-conjugated Annexin-V detection kit (640922, Biolegend) according to the manufacturer's instructions. C4-mDAPs were dissociated with Accutase and filtered through a 70 μm cell strainer to make single cell suspensions, washed with PBS twice, and incubated in binding buffer with Annexin-V-FITC and 7-AAD, Annexin-V-FITC and APC-hNCAM or Annexin-V-FITC and PE-TH for 15-30 min in the darkness. The reaction was stopped by adding 4 volumes of binding buffer, washed with PBS twice, and analyzed by flow cytometry. For the gating strategy used in the cell death assay, see FIG. 7a.

### Mixed Lymphocyte Reaction (MLR)-Like Co-Culture Assay

PBMC (C4 or K2) and C4-mDAPs were co-cultured at 1:1, 1:2, or 1:10 ratio (C4-mDAP: PBMC) in 96-well plate for 3-5 days. Dynabeads™ Human T-Activator (†) was used as positive control. PBMCs were then harvested, stained with APC-hCD3 and PE-hCD69 antibodies, and analyzed by flow cytometry to determine the proliferation profiles. C4-mDAPs were harvested, stained with APC-hNCAM antibody and Annexin-V-PE, and analyzed by flow cytometry to determine the cell death profiles. C4-mDAPs with or without C4-T_{REG} were co-cultured with T_{REG} suppression inspector (130-092-909, Miltenyi Biotech) for 7 days under in vitro inflammatory conditions. C4-mDAPs with Treg cells (+Treg suppression inspector) or without Treg cells, and anti-TGFβ1 or mouse IgG1 isotype control antibody were co-cultured for seven days.

### ELISA

After co-culture of PBMC and C4-mDAP for 3-5 days, an aliquot of culture supernatant was obtained to measure the amount of secreted IFN-γ by ELISA according to the manufacturer's instructions (DIF50, R&D Systems).

### Quantitative Real-Time PCR

Total RNA was isolated from C4-mDAPs with a GeneJET RNA Purification

Kit (Thermo Fisher Scientific) according to the manufacturer's instructions. cDNA synthesis was performed with an iScript™ cDNA Synthesis Kit (Bio-Rad). The following primers were used:

TH
forward:
(SEQ ID NO: 1)
5′-CGGGCTTCTCGGACCAGGTGTA-3′;
reverse:
(SEQ ID NO: 2)
5′-CTCCTCGGCGGTGTACTCCACA-3′;
Foxa2
forward:
(SEQ ID NO: 3)
5′-GGTGCTTTGGCTGACTTTTT-3′;
reverse:
(SEQ ID NO: 4)
5′-GTTGCTCACGGAGGAGTAGC-3′;
Lmx1a
forward:
(SEQ ID NO: 5)
5′-AGGGTGACGAGTTTGTCCTGA-3′;
reverse:
(SEQ ID NO: 6)
5′-CATCGCTTTTGCCTGAGTCTG-3′
and
Actin
forward:
(SEQ ID NO: 7)
5′-CATGTACGTTGCTATCCAGGC-3′
reverse:
(SEQ ID NO: 8)
5′-CTCCTTAATGTCACGCACGAT-3′.

For qRT-PCR, we used SsoAdvanced™ Universal SYBR Green Supermix and reactions were performed on a CFX Connect™ Real-Time System (Bio-Rad, Hercules, CA). Calculations of relative expression were performed by using the comparative Ct method, using the C4 fibroblast as a reference control (value=1).

### Western Blot

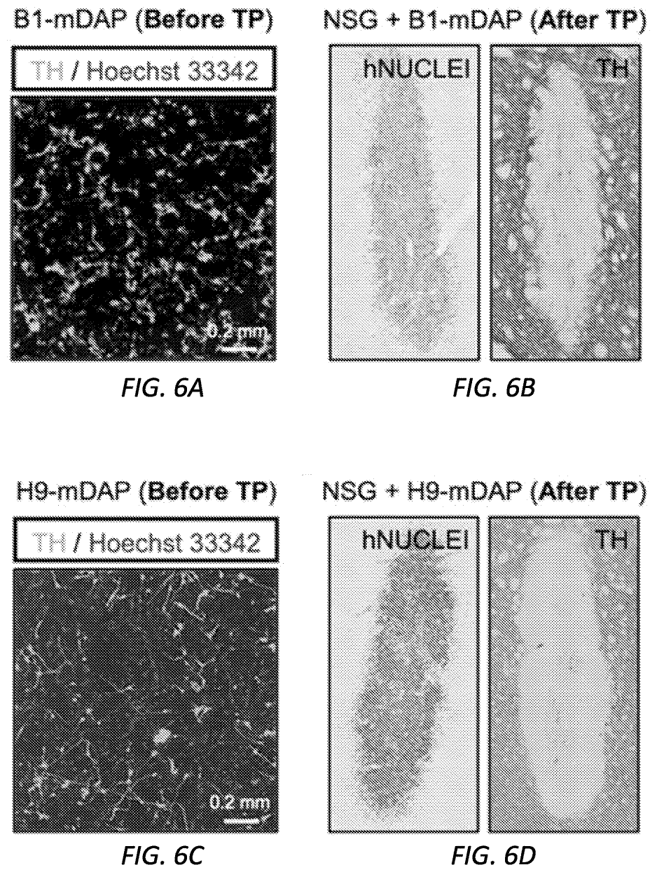

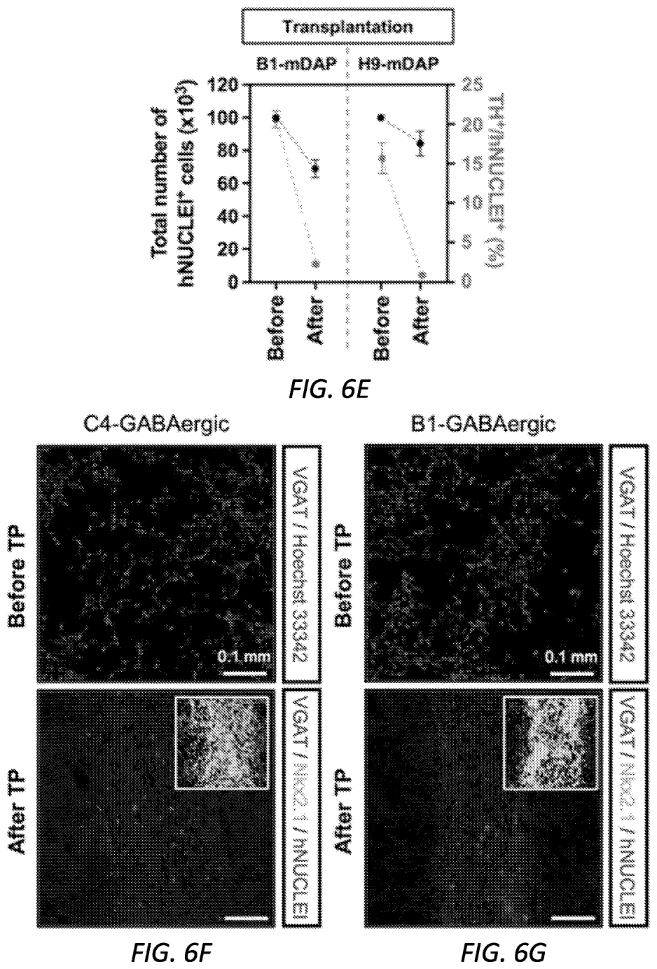

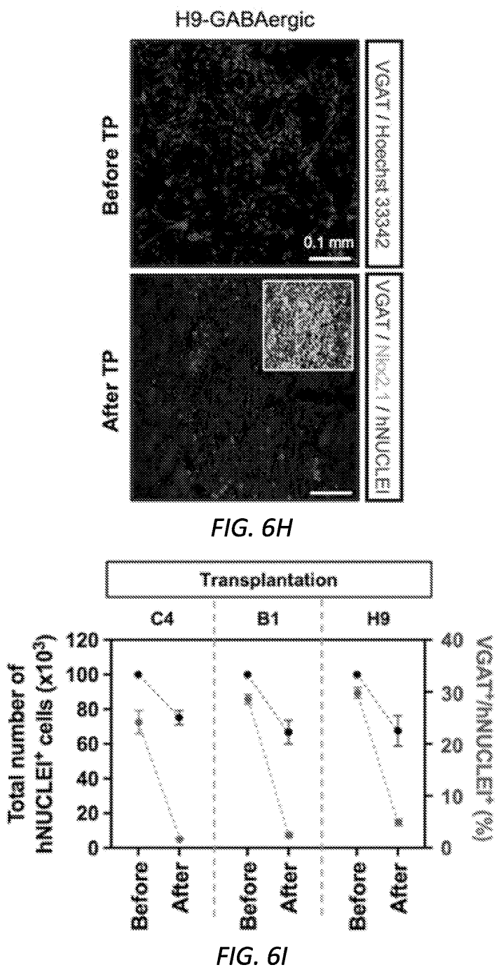

Cells were lysed in RIPA lysis buffer (Sigma) (with protease inhibitors) and protein concentration determined using the BCA assay (Thermo Fisher Scientific). An equal volume of 6× loading buffer was added to each sample, which were then boiled for 10 min and loaded onto a 4-12% Bis-Tris Plus gel. Proteins were electrophoresed and then transferred to polyvinylidene difluoride membrane. The membrane was probed with anti-Cleaved Caspase-3 (9661S, CST), anti-Caspase-3 (14220T, CST), anti-Zg16 (NBP1-58007, Novus Biologicals), anti-Hormad1 (13917, Proteintech), or anti-β-actin (ab8227, Abcam) antibody diluted 1:1000 in blocking solution (PBS containing 0.1% BSA) and subsequently incubated with horseradish peroxidase-conjugated secondary antibodies (Amersham). Bound antibodies were visualized using ECL (Amersham). For gel source data, see FIGS. 6a-6i.

### Immunocytochemistry

C4-mDAPs cultured in 6, 12, 24, 48-well plate were treated with 100 ng/ml IFN-γ, 20 ng/ml TNF-α, or 10 ng/ml IL-1β for 2 and 7 days, washed with PBS and fixed with 4% formaldehyde in PBS for 10 min. Cells were incubated for 1 h in blocking solution (0.3% Triton X-100 and 1% horse serum in PBS) at RT and then incubated overnight with anti-TH antibody in the same solution at 4° C. Cells were then incubated with Alexa Flour 488 or 568-conjugated secondary antibodies with Hoechst 33342 or DAPI for nuclei staining at RT for 1 h. After obtaining cell images by fluorescence microscopy (KEYENCE, Osaka, Japan), data regarding specific cell populations were determined from microscopic images using ImageJ software.

### Immunohistochemistry

After completion of experiments, animals were induced into deep anesthesia by intraperitoneal injection of ketamine/xylazine, followed by intracardiac perfusion with ice-cold PBS for 10 min and then 4% formaldehyde for 20 min, at a flow rate of 10 ml/min. Brains were removed and post-fixed overnight in 4% formaldehyde at 4° C. and then continuous incubation in 20% and 30% sucrose. Sucrose-infused brains were embedded in the OCT compound, frozen at −83° C., and coronal sections (30 μm) covering the entire striatum were serially collected (Leica CM1950, Buffalo Grove, IL). Free-floating coronal sections (including needle track or whole graft) were incubated in blocking solution (0.3% Triton X-100 and 1% horse serum in PBS) for 1 h and then were incubated with anti-MHCII (554926, BD), anti-Ibal (ab178846, Abcam), anti-TH (P40101, Pel-Freez Biologicals), anti-hNCAM (sc-106, Santa Cruz), and anti-hNUCLEI (ab191181, Abcam) antibodies overnight at 4° C. Stained brains were washed and stained with peroxidase-conjugated secondary antibody for 1 h. Finally, sections were visualized with the DAB peroxidase substrate kit following the manufacturer's instructions. Images were selected where the needle track or graft was clearly visible.

### Immunofluorescence

Free-floating coronal brain sections were processed for antigen retrieval as needed and pre-incubated in blocking solution (0.3% Triton X-100, 5% normal donkey serum, and 3% BSA in PBS) at RT for 1 h. Brain sections were incubated with anti-IFN-γ (ab216642, ab9657, Abcam) (506702, BioLegend), anti-hIbal (ab178680, Abcam), anti-hCD4 (ab133616, Abcam), anti-hCD45 (ab40763, Abcam), anti-Foxp3 (ab22510, Abcam), anti-TH (AB1542, Sigma-Aldrich), anti-CD11b (MA180560, Thermo Fisher Scientific), anti-CD11c (PA5-90208, Thermo Fisher Scientific), anti-NKp46 (MABF1970, EMD Millipore), anti-CD19 (14-0194-82, Thermo Fisher Scientific), anti-rCD4 (ab237722, Abcam), anti-rCD8 (550298, BD), anti-HLA-ABC (ab70328, Abcam), anti-HLA-DR (ab92511, Abcam), anti-FoxA2 (89000721, Fisher Scientific), anti-Ki67 (ab16667, Abcam), anti-CD47 (ab260419, Abcam), anti-NeuN (ab279297, Abcam), anti-hGFAP (DPABY-745, Creative Diagnostics), anti-hCOL1A1 (AF6220, R&D Systems), anti-OLIG2 (AB9610, Sigma-Aldrich), anti-VGAT (PA5-63808, Thermo Fisher Scientific) and anti-Nkx2.1 (MA5-33074, Thermo Fisher Scientific) antibodies in the same solution overnight at 4° C., washed 3 times with PBS, and then incubated with Alexa Fluor 488-, Alexa 568-, or Alexa 647-conjugated secondary antibody with DAPI or Hoechst 33342 for 1 h at RT. Sections stained with secondary antibodies alone were processed and photographed under the same conditions and used as negative controls.

### Statistical Analysis

Microsoft Excel software (Microsoft Corp.) and GraphPad Prism v9 software were used for all statistical analyses and the specific tests used are described in the figure legends. All experiments were performed in biological triplicate unless otherwise indicated and the exact number of samples (n) used can be found in the figure legends. Two investigators, blinded to experimental group assignments, counted the number of stained cells or MFI in all brain sections of each mouse/rat or in the most visible section of each mouse/rat's′ needle track. Student's t-test (unpaired two-tailed) was used when comparing two groups that all data passed the Shapiro-Wilk normality test or within a group while multigroup comparisons were performed using two-way ANOVA followed by Bonferroni post-hoc test, or one-way ANOVA followed by Tukey's test. A value of p<0.05 was considered statistically significant, and the asterisk in the figure indicates the significance of the p value: * p<0.05; ** p<0.01; *** p<0.001.

### Example 1: Rapid and Preferential Loss of TH⁺mDA Neurons Following Cell Transplantation

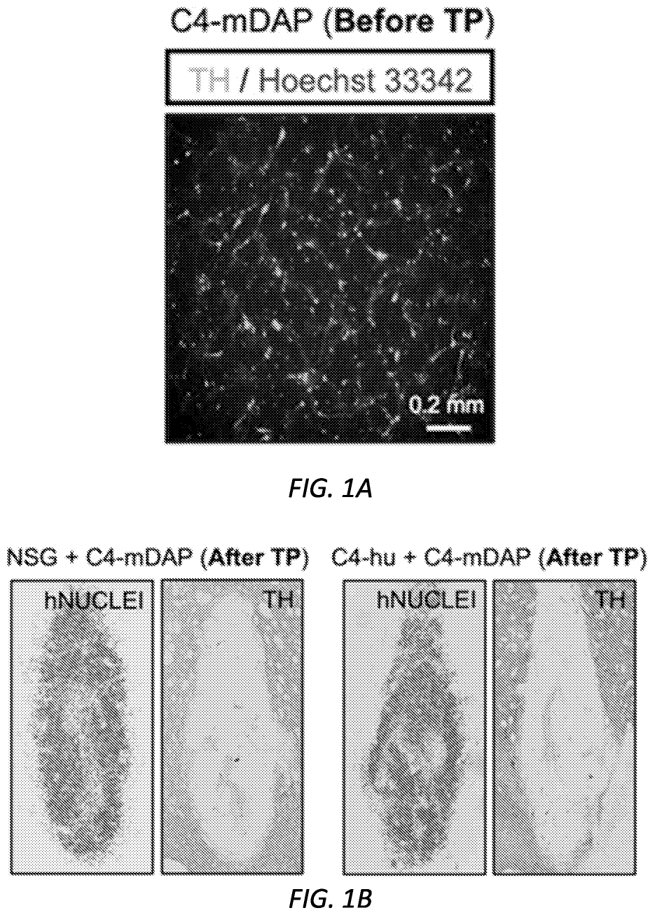

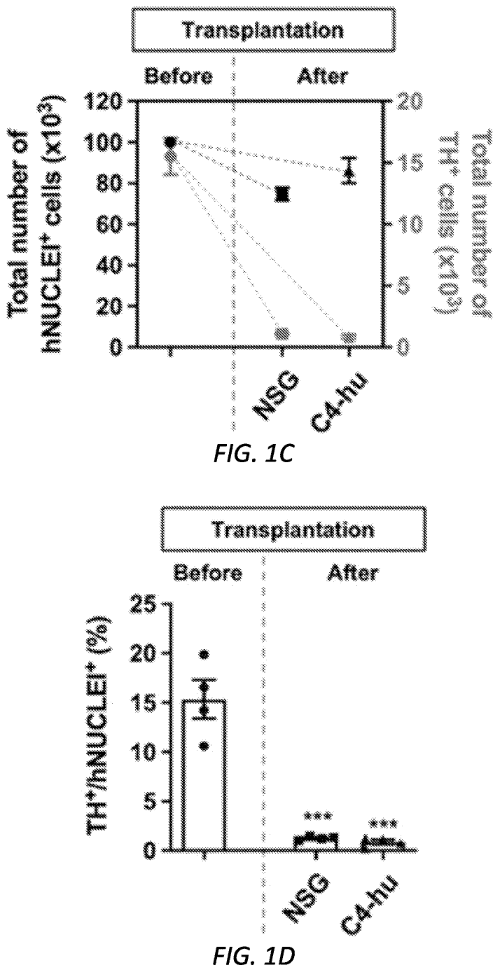

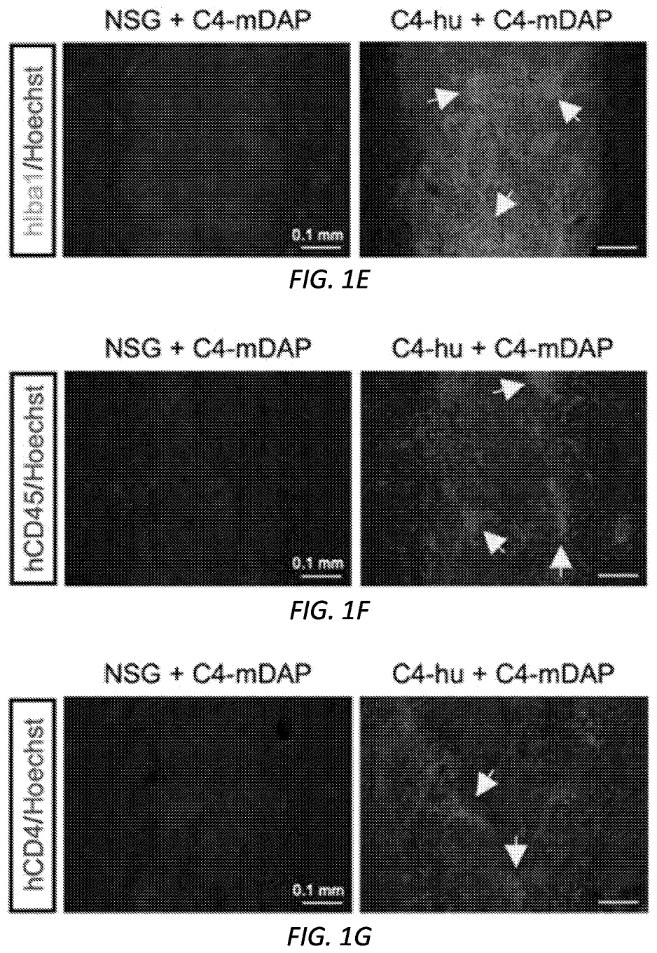

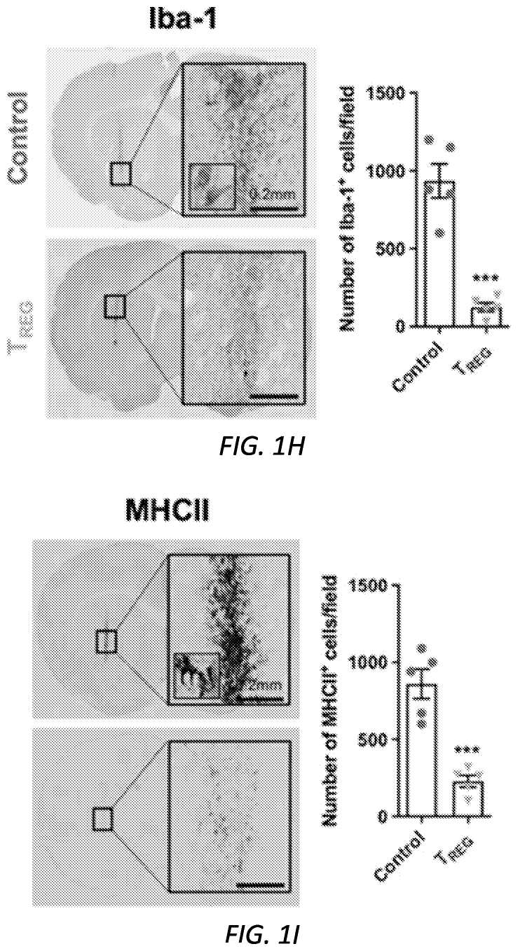

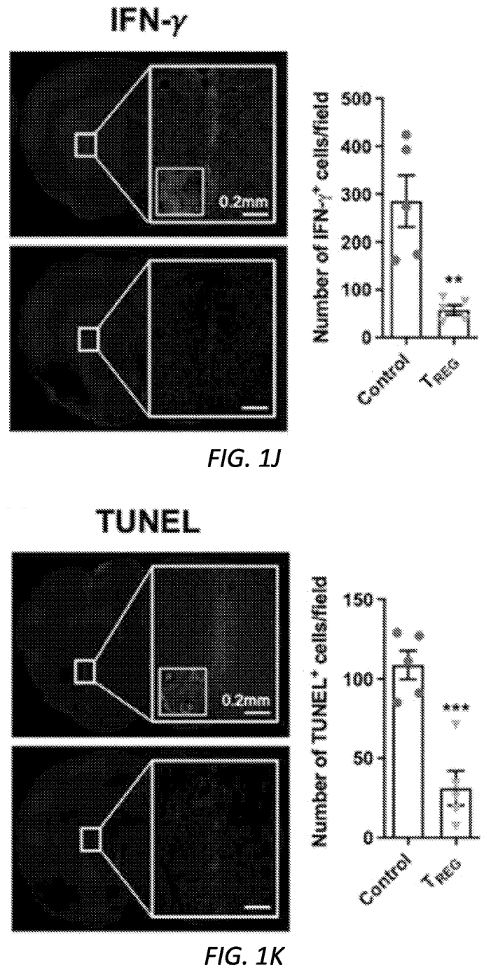

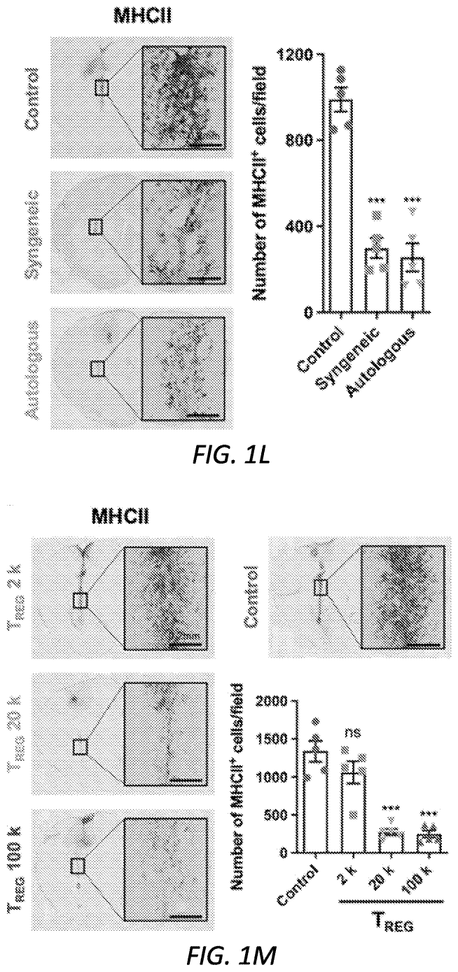

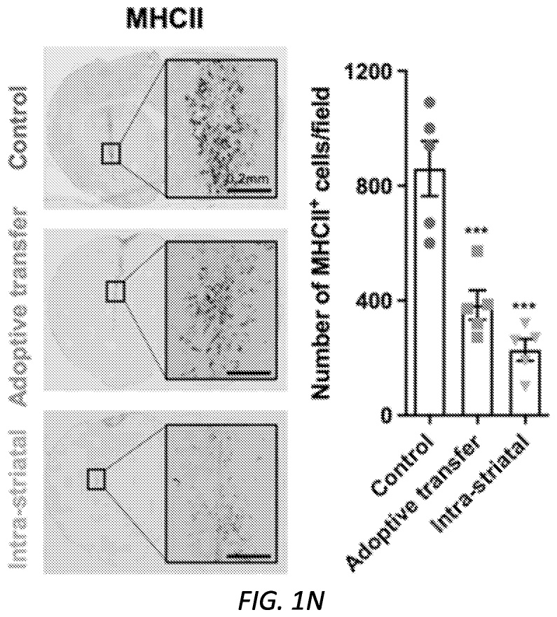

To model the early survival of human mDA neurons in an autologous setting, we transplanted a PD patient's (C4) iPSC-derived midbrain dopamine progenitor cells (mDAPs) into the striatum of mice humanized with immune cells autologous to the patient as described¹⁶. C4-iPSC were differentiated in vitro for 28 days using our established spotting-based procedure^{21,22}, generating C4-mDAPs containing approximately 15% TH⁺ mDA neurons (FIGS. 1a,1d), which is typical for iPSC-derived mDA differentiation protocols²¹⁻²⁴. We transplanted a total of 100,000 C4-mDAPs into the striatum of immunologically humanized mice (C4-hu; reconstituted with the patient's PBMC) as well as immunodeficient NOD SCID gamma (NSG) mice. Animals were sacrificed at 2 weeks post-transplantation and examined for graft survival by immunohistochemistry using antibodies against human nuclei (hNUCLEI) and against TH to detect the total number of surviving cells and TH⁺ mDA neurons, respectively (FIG. 1b). We detected 85,901±7,460 and 74,455±3,842 hNUCLEI⁺ cells in C4-hu and NSG mice, respectively (FIG. 1c). In sharp contrast, only 627±279 and 942±171 TH⁺ cells survived in C4-hu and NSG mice, respectively, representing<1% of transplanted cells (or <10% of transplanted mDA neurons). These data demonstrate that mDA neurons within the graft die disproportionately and at an early stage (i.e., within 2 weeks post-transplantation) even in relatively immunodeficient mice with or without human immune system reconstitution, while the rest of the transplanted cells are mostly spared. To address whether mDA neurons' vulnerability is unique to this patient (C4), we transplanted additional in vitro differentiated cells from an independent hiPSC line (B1; derived from a separate sporadic PD patient) and from the H9 hESC line and found that this phenomenon is a general feature (FIGS. 6a-6e). Notably, C4-hu mice showed higher numbers of human cells (hNUCLEI⁺) in the grafts compared to NSG mice (FIG. 1c), which may be due to immune cell infiltration from the humanized immune system as a secondary response to the initial neuroinflammation (as observed in traumatic brain injury (TBI)). In support of this, immunohistochemical analyses revealed that substantial numbers of immune cells from the humanized host immune system such as human Iba-1⁺ (hIba-1⁺; macrophage/microglia), hCD45⁺ (leukocyte) and hCD4⁺ (T lymphocyte) accumulated near the graft in C4-hu mice but not in NSG mice (FIG. 1e-1g). To address whether initial cell death is specific to mDA neurons, we tested another neuronal lineage, vesicular GABA transporter (VGAT)⁺ GABAergic neurons derived from two hiPSC (C4 and B1) and one hESC (H9) lines, and we found that these neurons similarly showed early cell death after transplantation (FIGS. 6f-6i). Thus, while more neuronal lineages need to be tested to generalize this conclusion, our data suggest the possibility that initial cell death following transplantation may be general to post-mitotic neurons.

### Example 2: Effects of Needle Trauma and Co-Transplantation of Autologous T_{REG} In Immunocompetent Rats

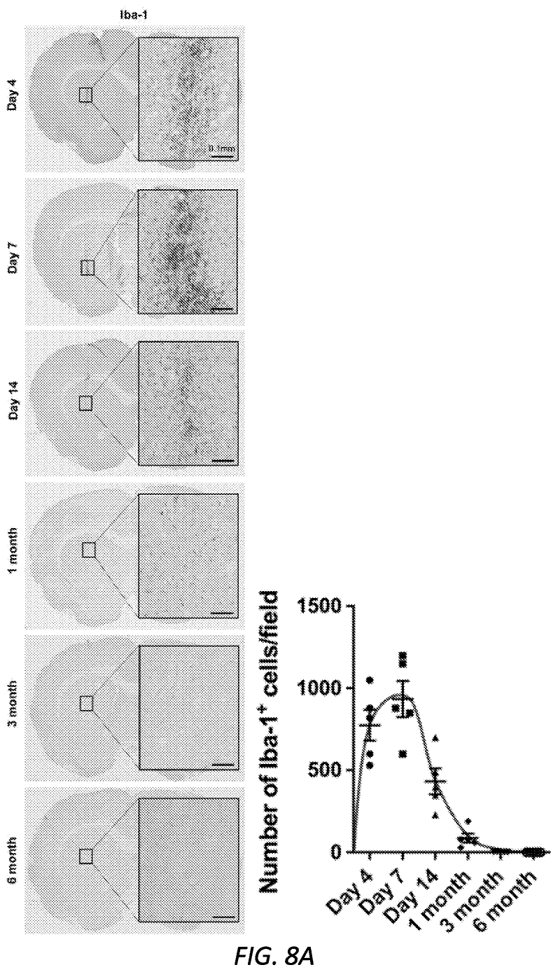

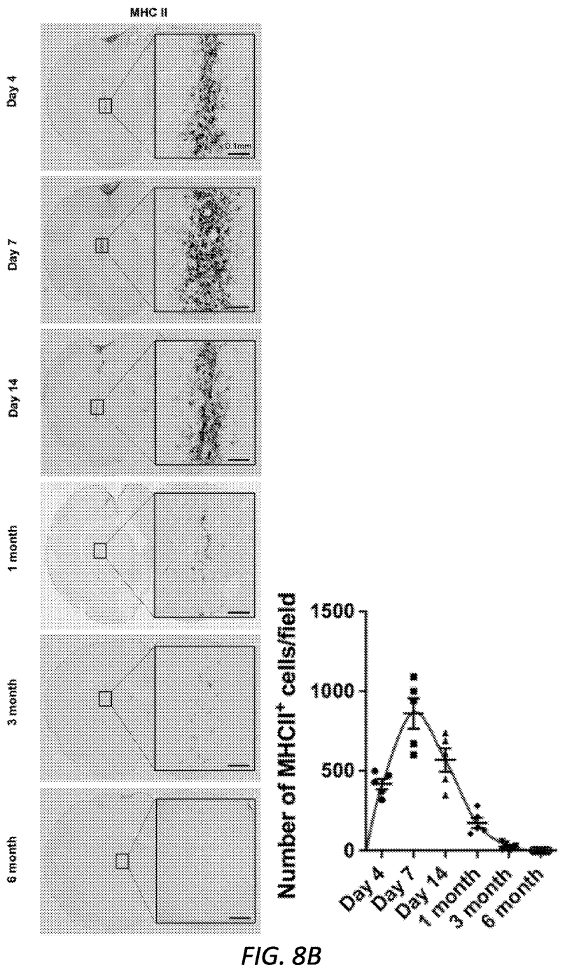

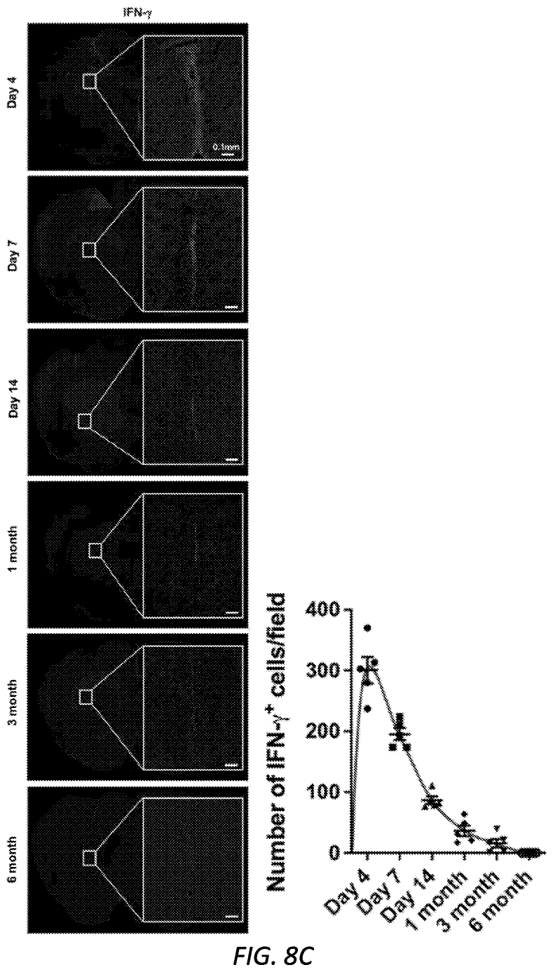

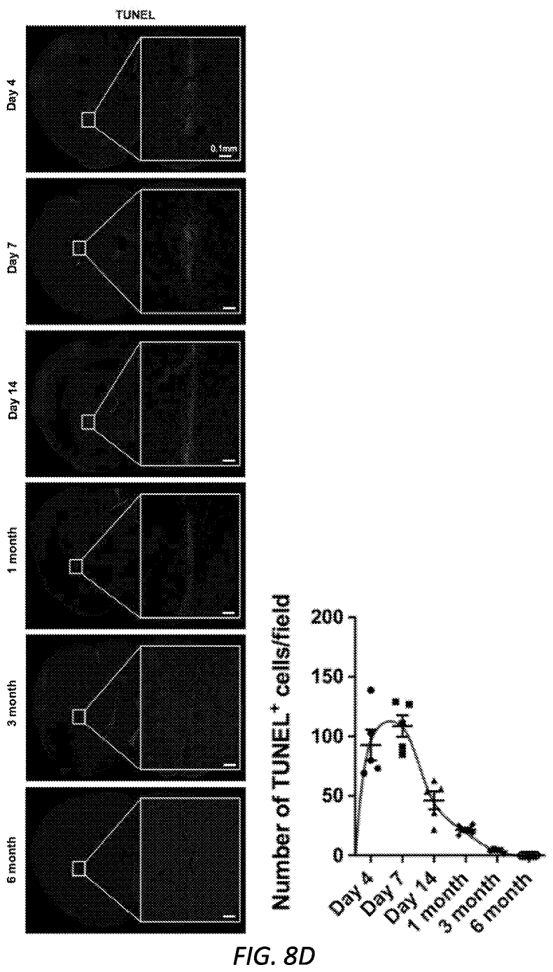

While the above data revealed important insights regarding the early events following transplantation, these immunodeficient mice may differ enough from real clinical situations to limit validity of immune response analyses. Thus, we used Fischer 344 rats to investigate the changes in the brain caused by surgical procedure. When we injected media only (without any cells) into the striatum of Fischer 344 rats, we found acute pro-inflammatory cytokine release and immediate activation of astrocytes/microglia at the injection site (FIGS. 7a-7d), indicating the host brain's response to penetrating brain injury (referred to here as “needle trauma”) reproduces the well-known effects of other forms of TBI using closed head injury models^{25,26}. We next analyzed the host response at different time points (4 days to 6 months post-injection) and found that Iba-1⁺ and MHCII⁺ inflammatory cells show robust infiltration near the needle track, reaching peak numbers at day 7 before declining at 1 month and becoming undetectable at 6 months (FIGS. 8a,8b). Notably, interferon-gamma (IFN-γ)⁺ cells peaked at day 4 post-injection, the earliest time point analyzed, indicating that needle trauma triggers immediate neuroinflammation in the host brain, which also gradually decreased over time (FIG. 8c). Needle trauma-induced death of host brain cells near the injection site, as examined by TUNEL staining, peaked at day 7 post-injection (FIG. 8d). Since needle trauma triggers substantial neuroinflammation-induced death of host brain cells, we speculated that implanted cells, particularly mDA neurons, may also be subject to neuroinflammation-induced death shortly after transplantation, consistent with our data (FIGS. 1b-1d; FIG. 8d) as well as with previous studies using VM cell transplantation^{7-10.}

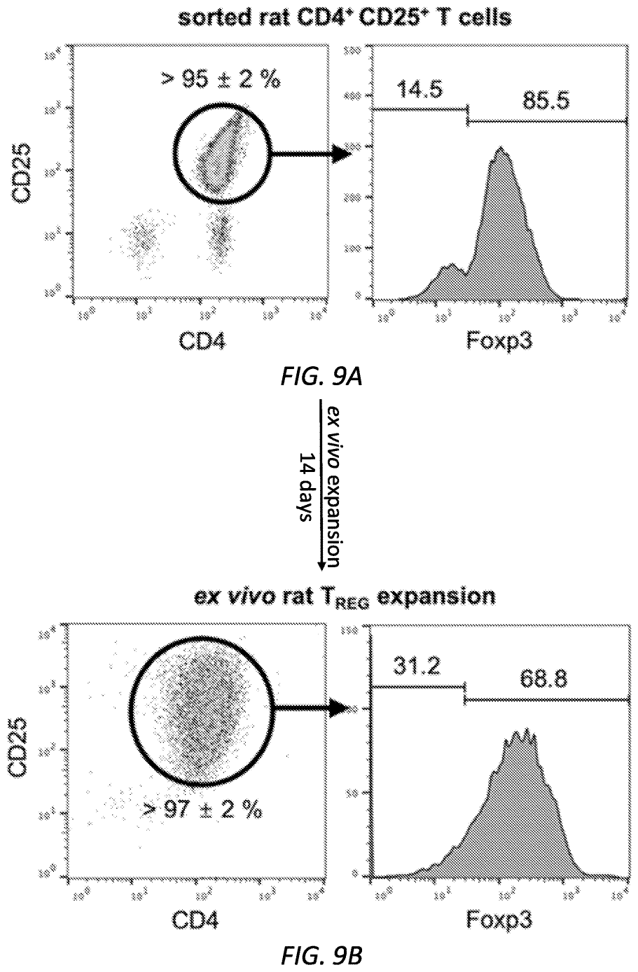

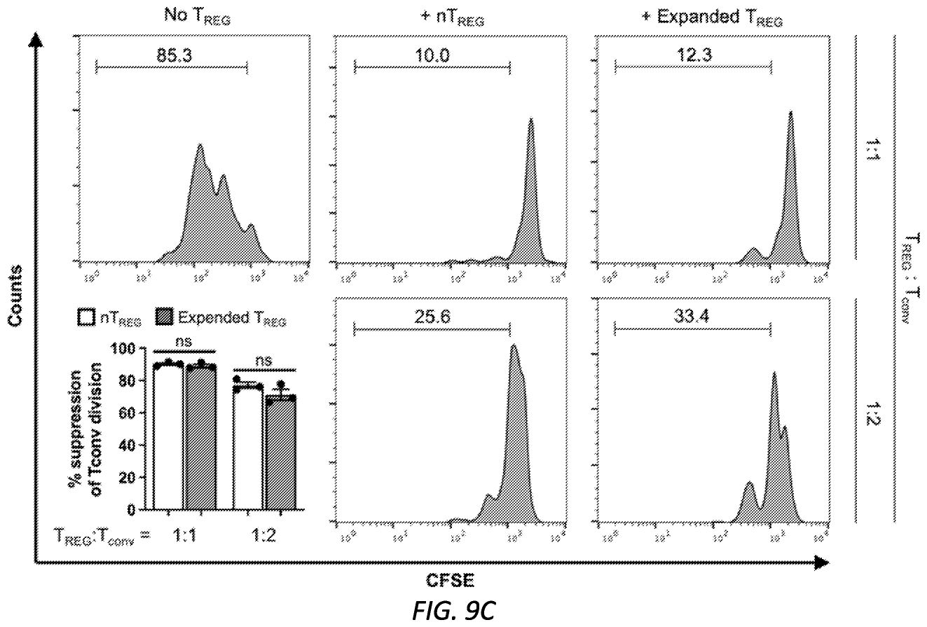

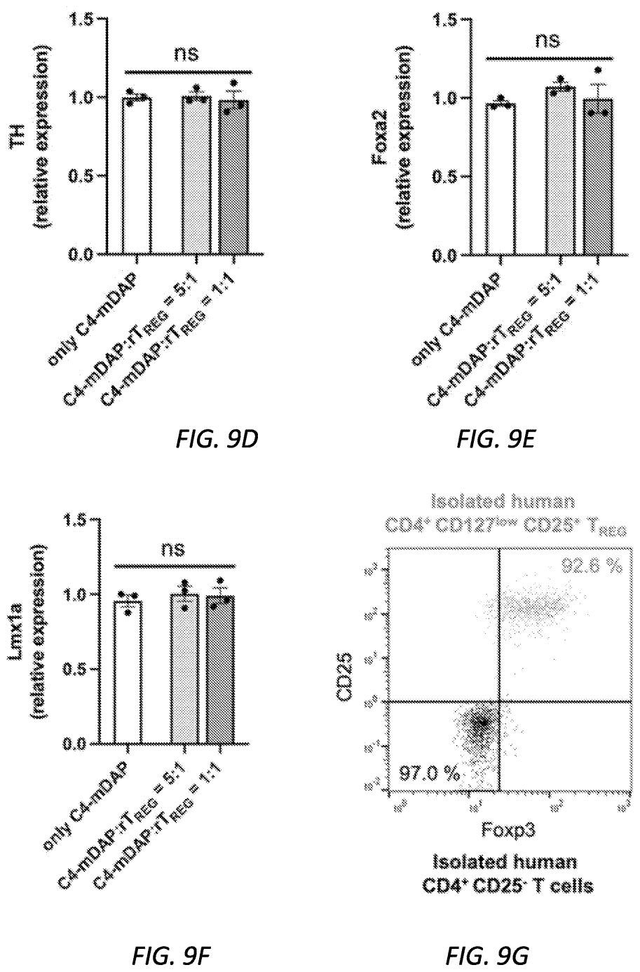

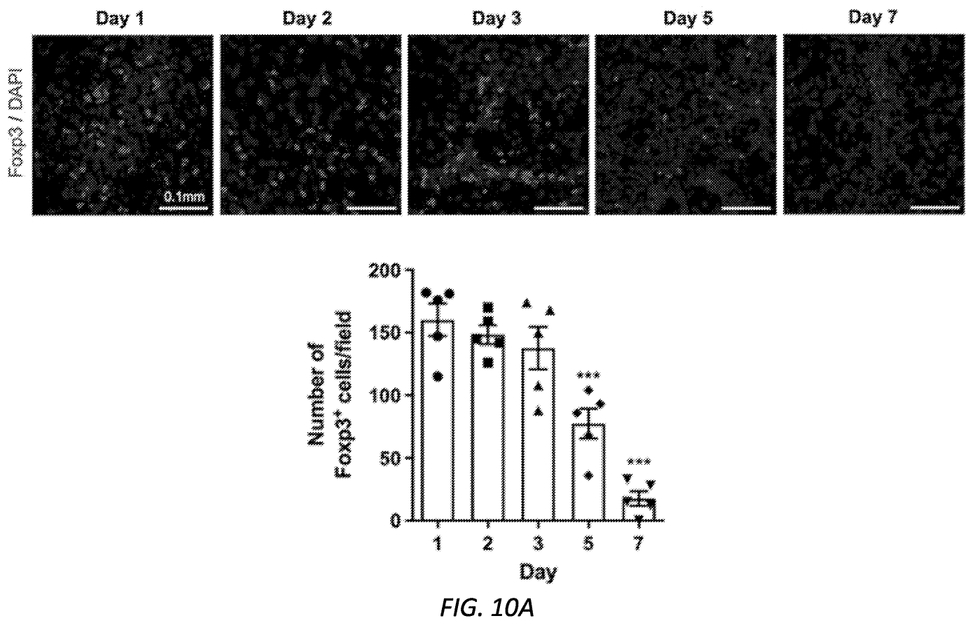

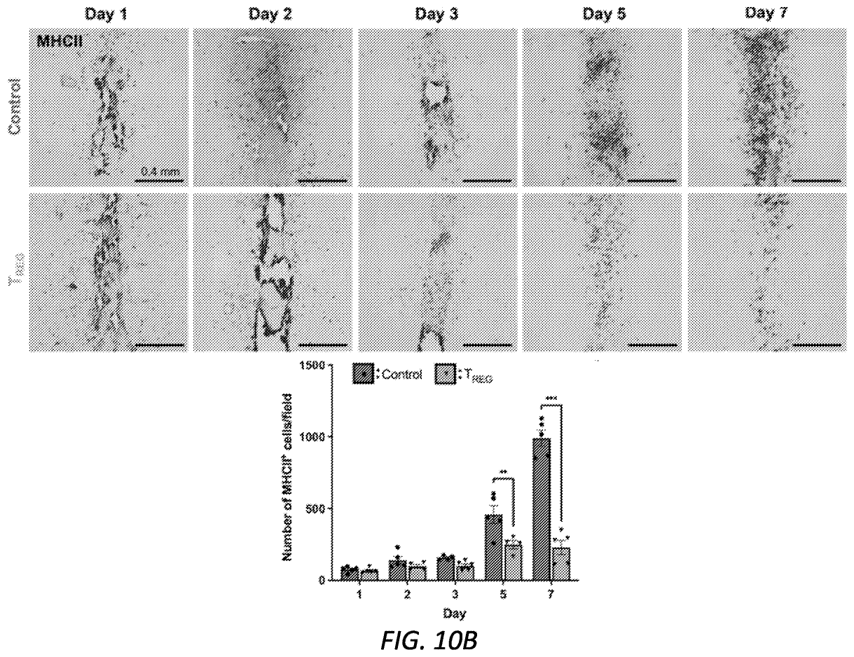

In TBI, it is well established that after the initial inflammatory cell infiltration, T_{REG} eventually infiltrate into the damaged area as a part of the repair process and return to homeostasis^{25,26}. Thus, we hypothesized that co-transplantation of autologous T_{REG} might be an effective approach to reducing the acute neuroinflammation caused by needle trauma. To address this, we isolated autologous CD4⁺CD25⁺ T_{REG} (Foxp3⁺, 85.5±3.12%) from each individual Fischer 344 rat by FACS sorting of peripheral blood obtained through the jugular vein (FIG. 9a), injected 20,000 cells into the striatum of the same rat, and analyzed each brain at day 2 or 7. Transplantation of T_{REG} significantly suppressed acute induction of pro-inflammatory cytokines (TNF-α, IL-1β) at day 2 and infiltration of inflammatory cells at day 7, associated with robust protection of host neurons from death compared to control groups (FIGS. 1h-1k, FIGS. 7e-7g), demonstrating their significant protective effects. We next compared the protective effects of autologous versus syngeneic T_{REG} and observed that rat autologous and syngeneic T_{REG} inhibited infiltration of MHCII⁺ inflammatory cells with similar efficiency (FIG. 1l). Autologous T_{REG} suppressed MHCII⁺ inflammatory cell infiltration in a dose-responsive manner, reaching its maximal effect when 20,000 cells were transplanted (FIG. 1m). Injected T_{REG} were initially detected near the needle track and gradually disappeared within a week, but effectively inhibited infiltration of MHCII⁺ inflammatory cells (FIGS. 10a-10b).

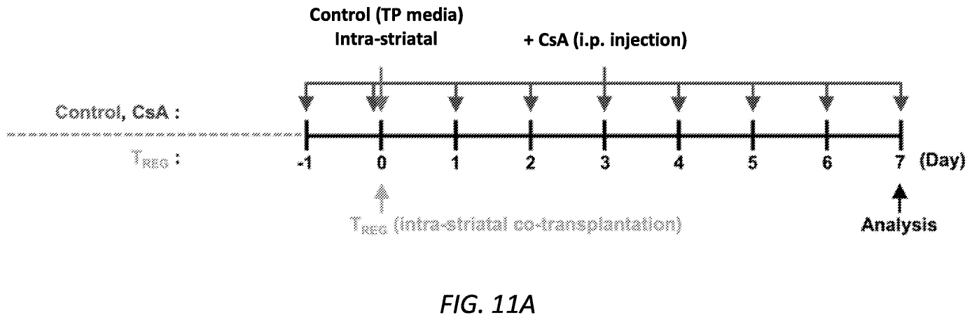

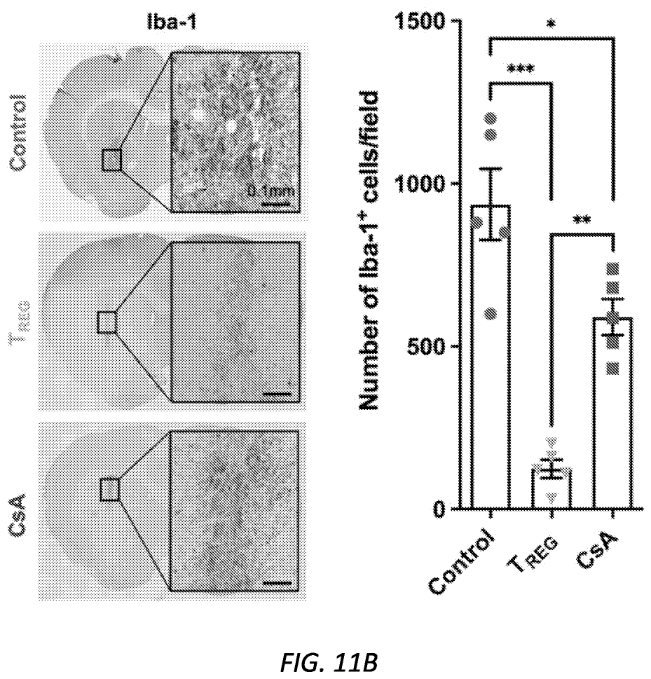

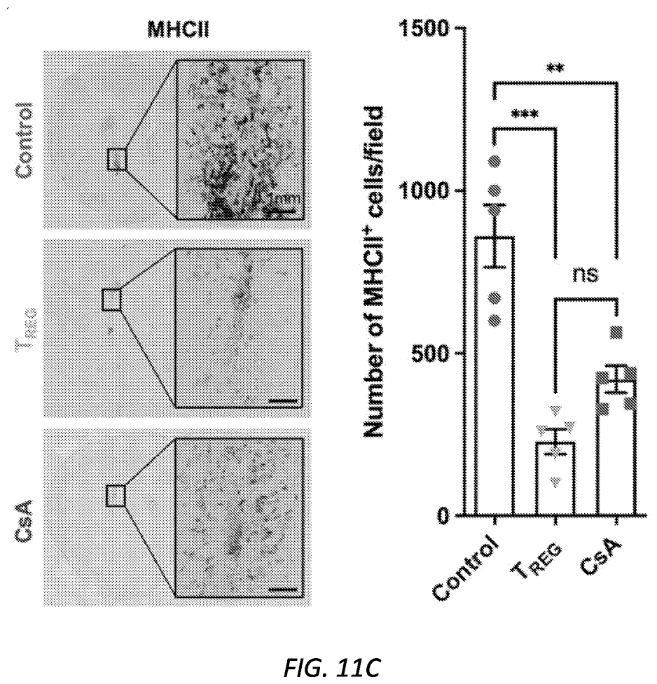

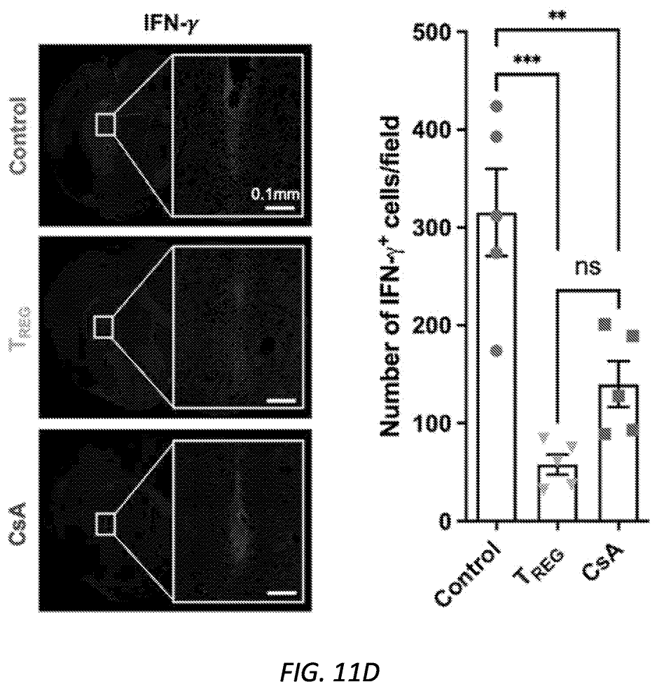

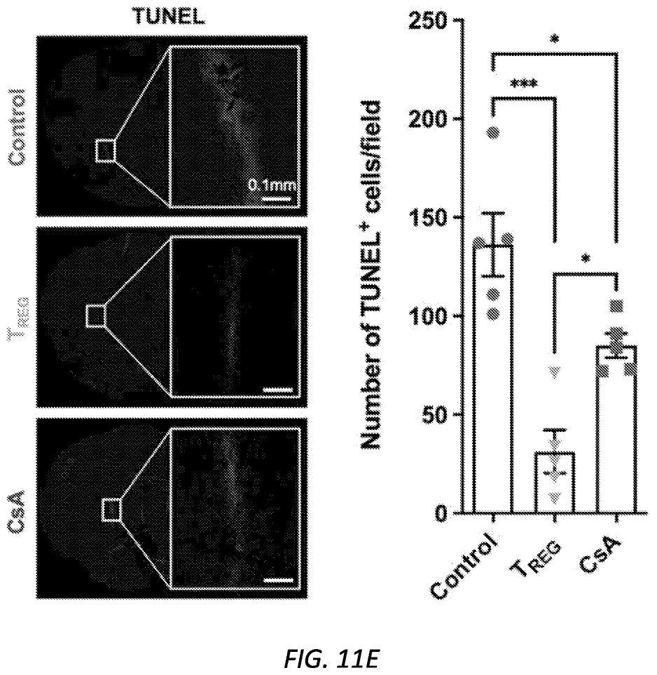

We also tested the effects of conventional T_{REG} therapy using adoptive transfer^{27,28} by intravenously injecting about 1,000,000 ex vivo expanded autologous CD4⁺CD25⁺ T_{REG} (Foxp3⁺, 68.8±4.07%) (FIG. 9b). Although adoptive transfer reduced infiltration of MHCII″ cell by approximately 50%, it was less efficient than direct intra-striatal transplantation of 20,000 T_{REG} (FIG. 1n). Notably, there was no functional difference between natural T_{REG} (nT_{REG}) and expanded T_{REG} (FIG. 9c). Finally, we tested whether administration of cyclosporine A (CsA), a typical immunosuppressant, can suppress the inflammatory effects of needle trauma. When administered daily until the animals were sacrificed, CsA treatment significantly inhibited host inflammatory response (e.g., hIba-1⁺, MHCII⁺, IFN-γ⁺, and TUNEL⁺ cells) but was not as effective as the single T_{REG} transplantation (FIGS. 11a-11e).

### Example 3: Analysis of Immunogenicity In Vitro

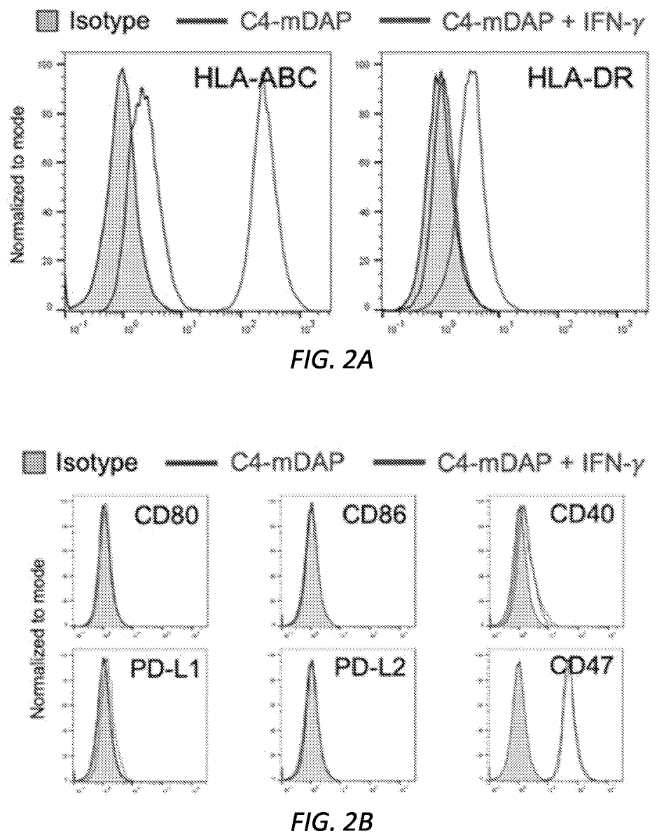

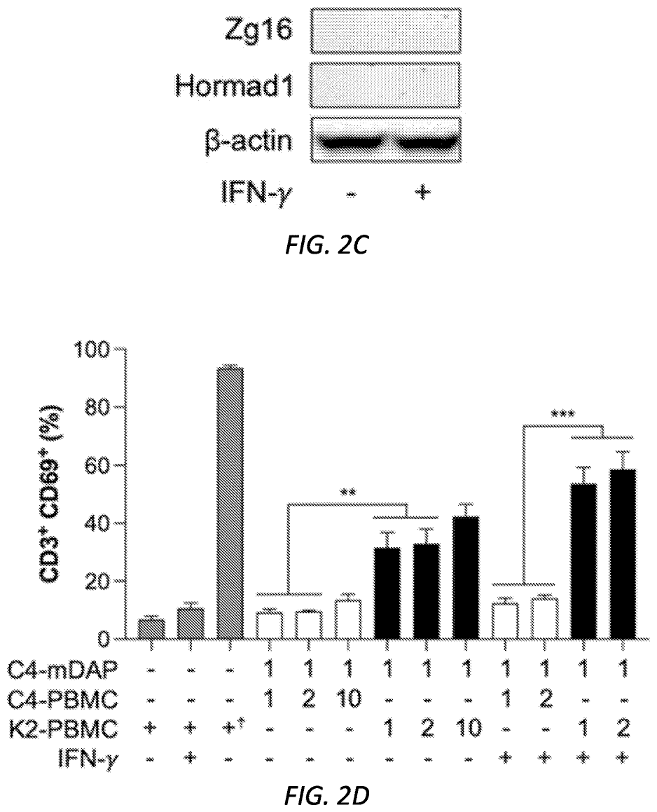

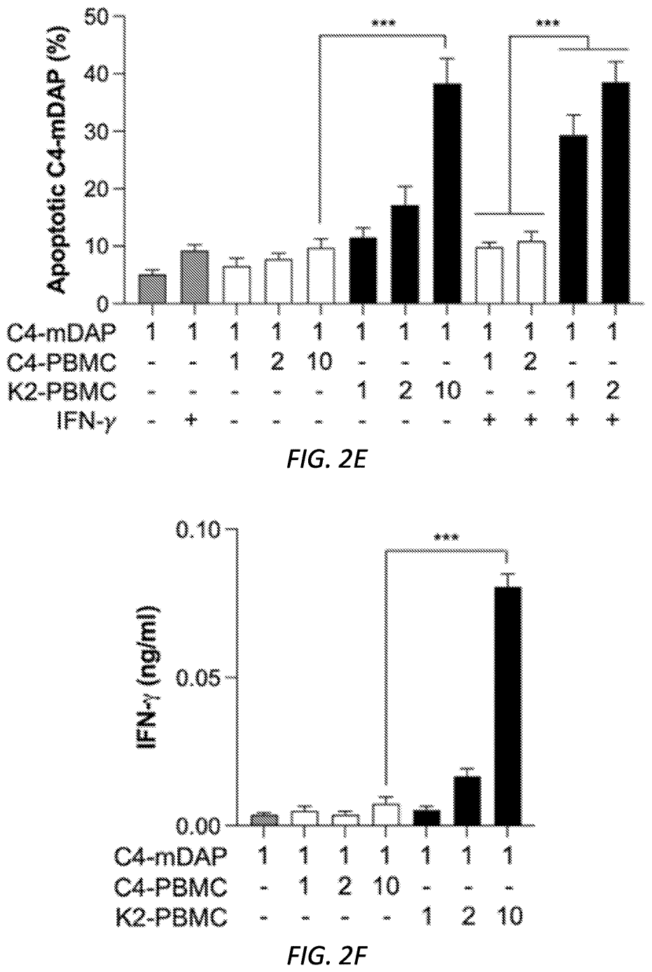

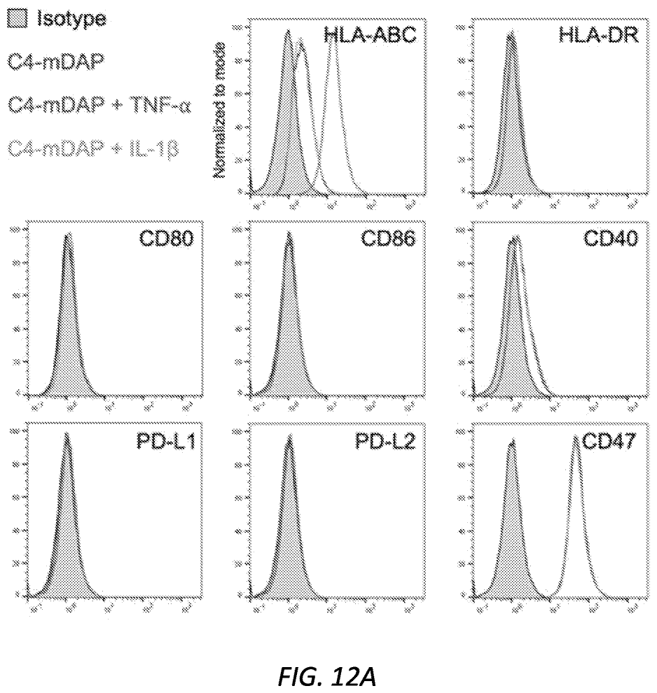

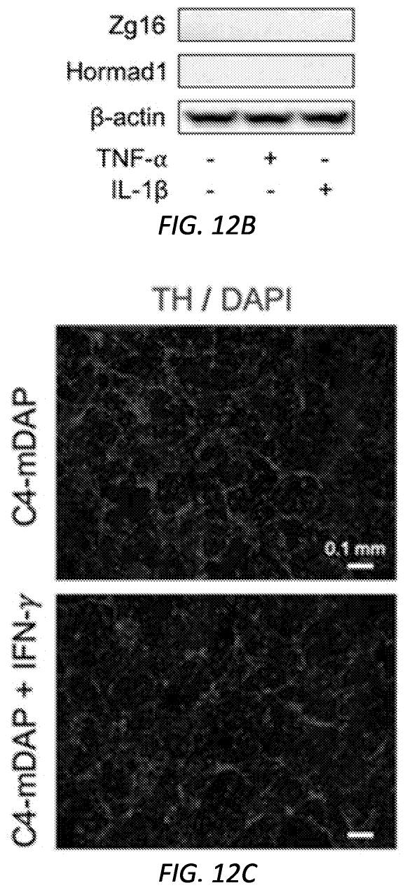

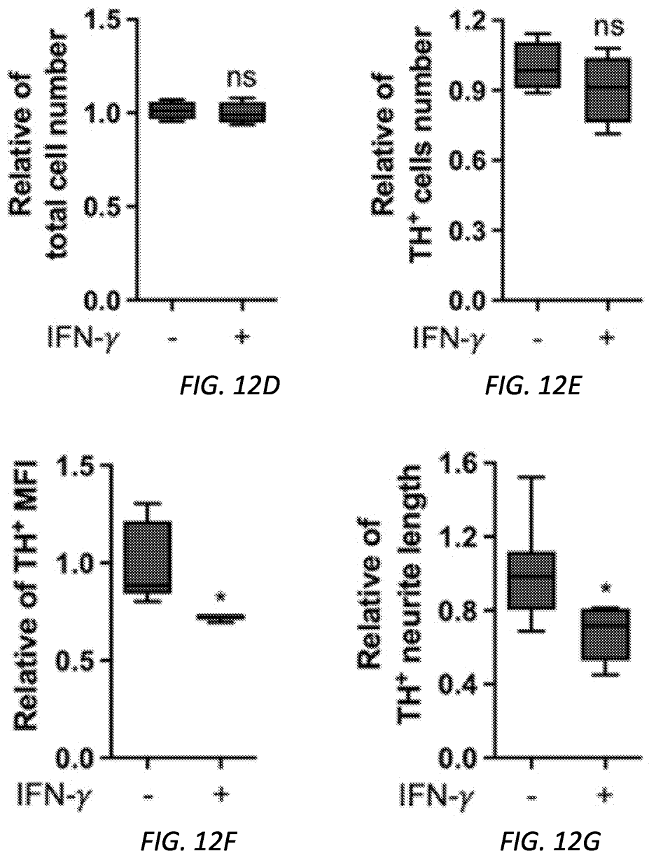

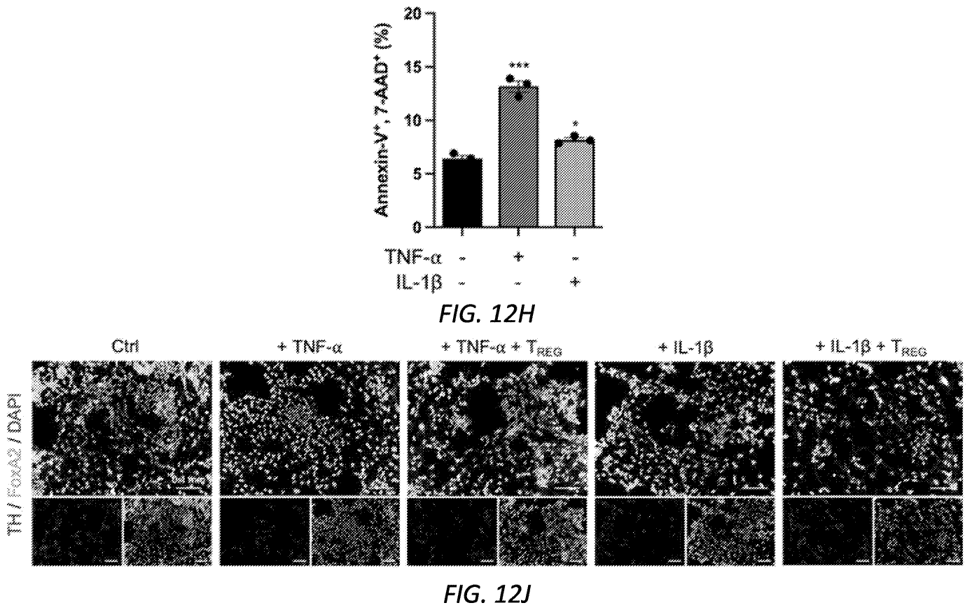

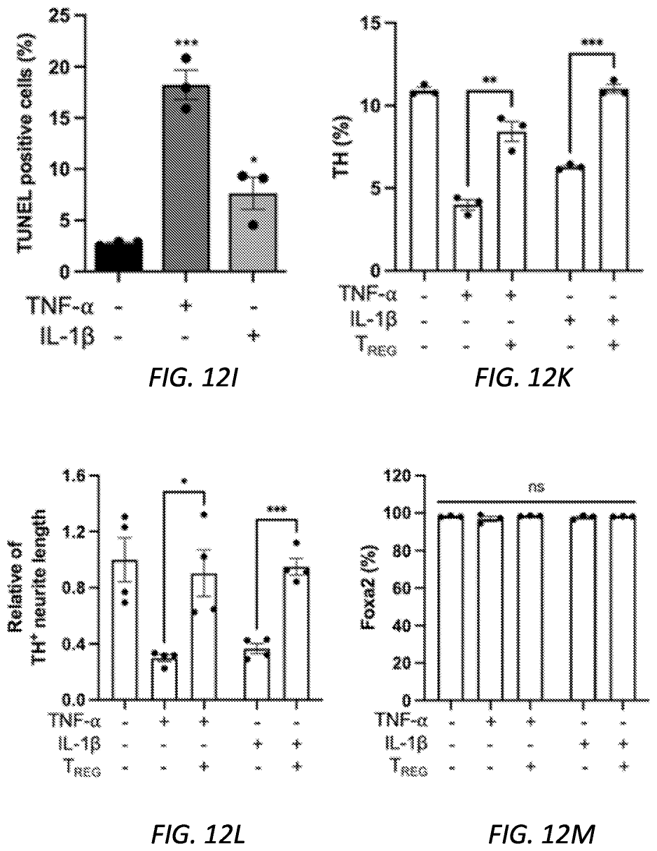

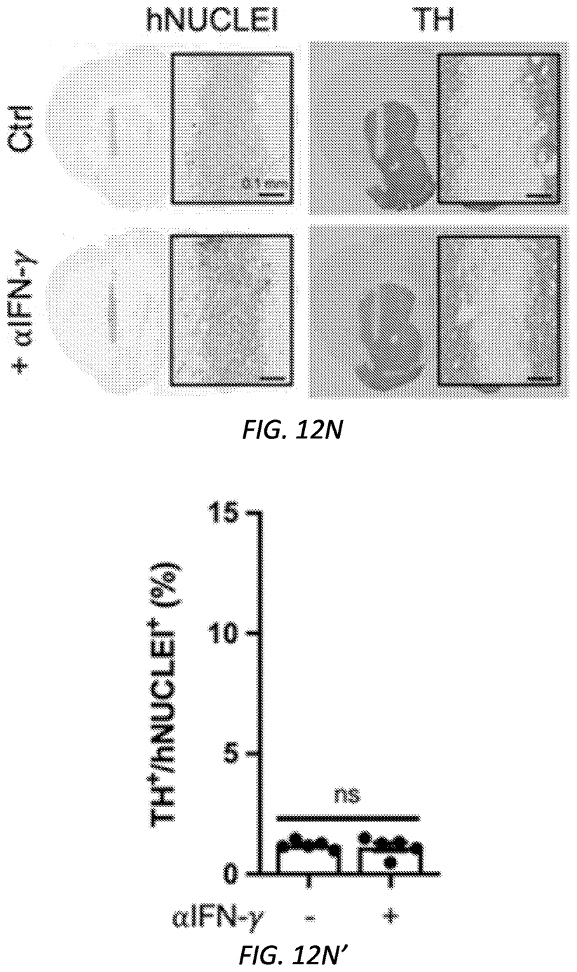

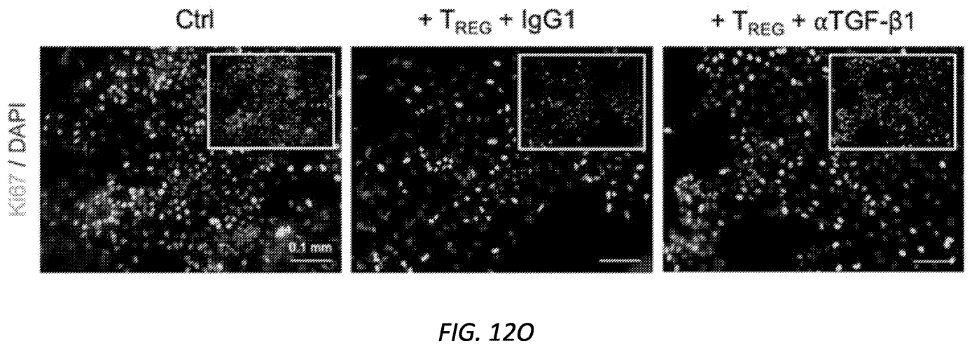

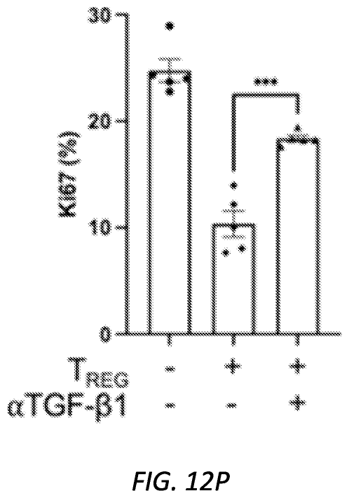

To investigate the effects of neuroinflammation in greater detail, we treated C4-mDAPs^{16,21} with pro-inflammatory cytokines (TNF-α, IL-1β, and IFN-γ). First, we examined expression of molecules related to immunogenicity²⁹⁻³¹ and found that HLA class I and/or II molecules were robustly induced following treatment for 2 days with IFN-γ (FIG. 2a) or TNF-α, but not with IL-1β (FIG. 12a). In contrast, expression of T cell co-stimulatory molecules (CD80, CD86, and CD40) and immune checkpoint molecules (PD-L1, PD-L2 and CD47) were unaltered by treatment with these cytokines (FIG. 2b, FIG. 12a). In addition, expression of immunogenic factors Zg16 and Hormadl^{32,33} was neither detected nor induced (FIG. 2c, FIG. 12b), suggesting that pro-inflammatory cytokines do not significantly change the expression of immunogenicity-related molecules in C4-mDAPs except for HLA molecules. We next analyzed immune responses in vitro by mixed lymphocyte reaction (MLR)-like co-culture assays using autologous or allogeneic PBMC. When C4-mDAPs and allogeneic PBMC (K2-PBMC) were co-cultured, T cell activity was prominently induced, and was further enhanced by IFN-γ treatment (FIG. 2d). In addition, significant apoptotic cell death of C4-mDAPs occurred when co-cultured with K2-PBMC (FIG. 2e). In sharp contrast, T cell activity was not induced when co-cultured with autologous PBMC (C4-PBMC), either in the absence or presence of IFN-γ, and no cell death was observed even in the presence of IFN-γ. Furthermore, enzyme-linked immunosorbent assay (ELISA) confirmed that incubation of C4-mDAPs with allogeneic, but not autologous, PBMC triggers IFN-γ secretion (FIG. 2f). Together, our data show that, when co-incubated with C4-mDAPs, allogeneic, but not autologous, PBMC triggers T cell activation, which is consistent with our previous findings of rejection/survival patterns in allogeneic and autologous transplantation studies using humanized mice¹⁶. Thus, we conclude that early death of mDA neurons was not caused by immunogenicity of the transplanted autologous mDAPs.

### Example 4: Effects of Pro-Inflammatory Cytokines on mDAPs In Vitro

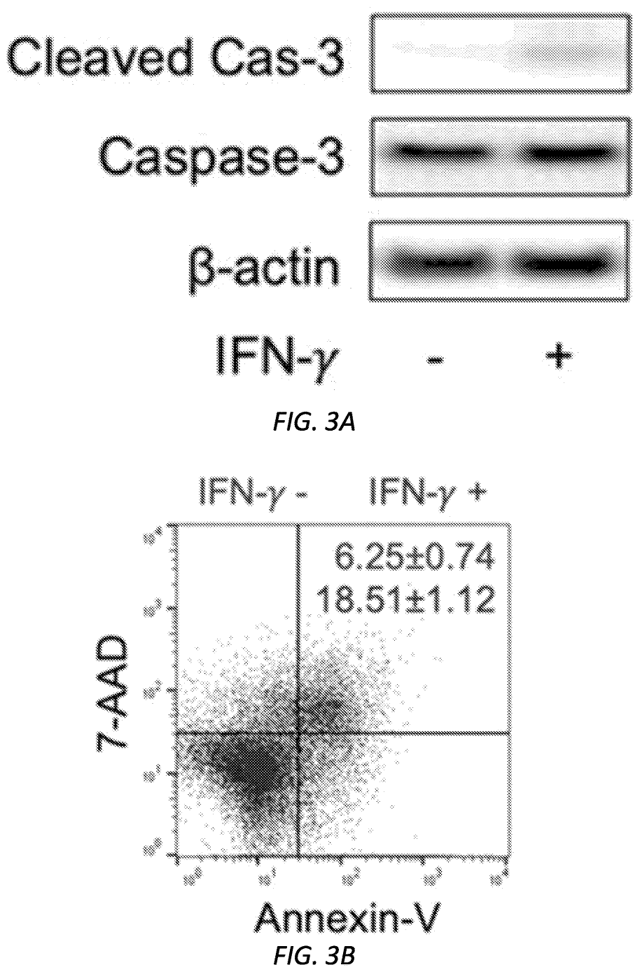

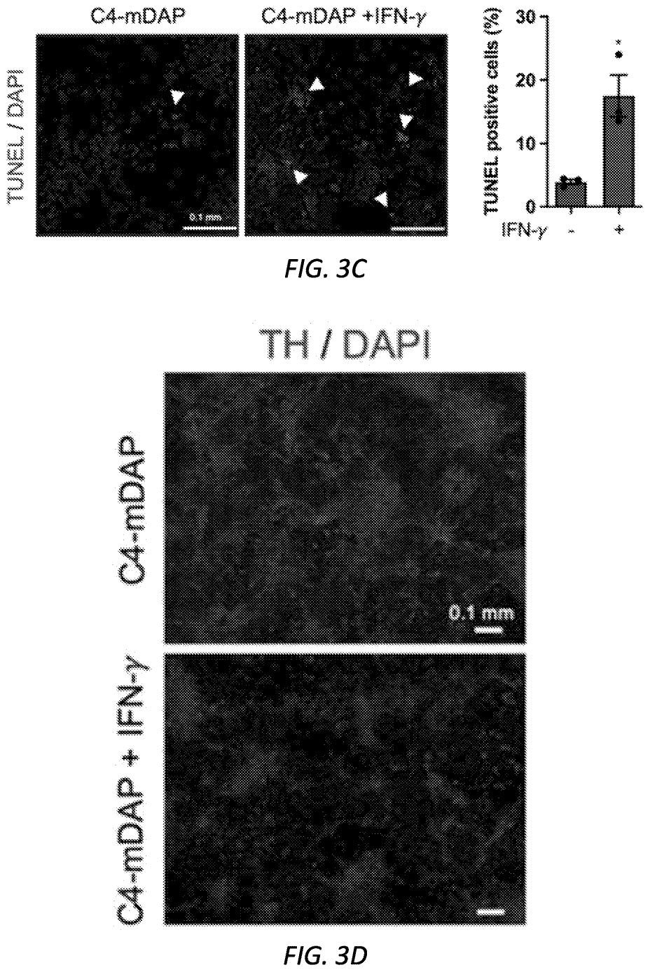

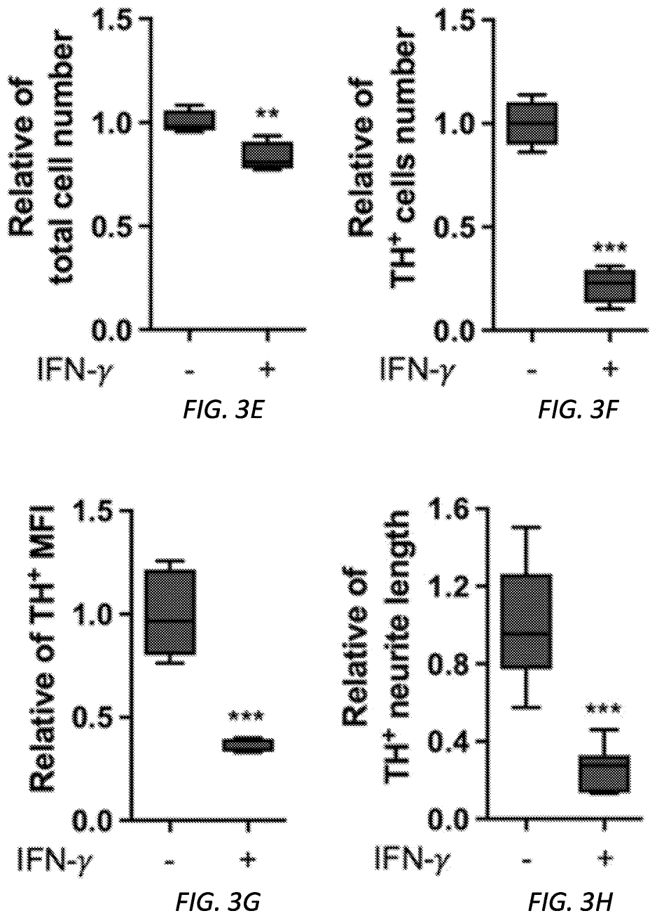

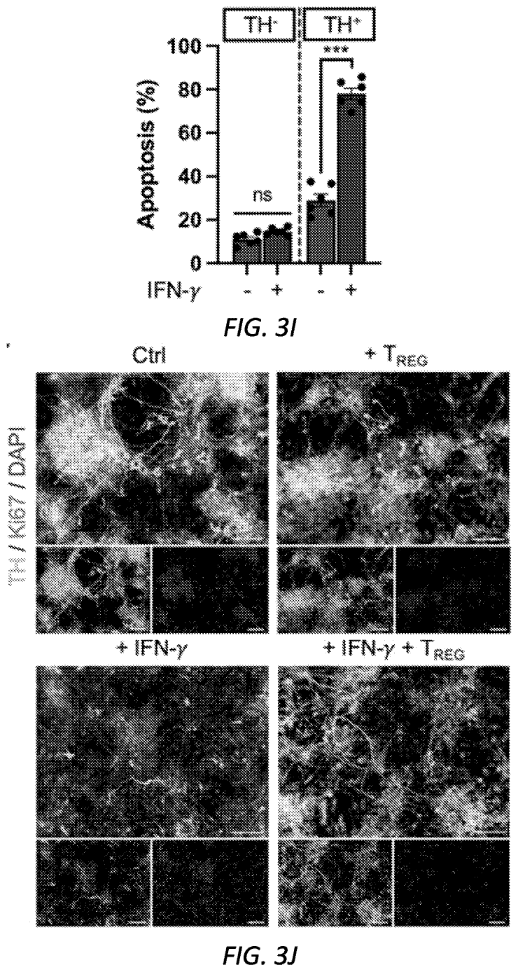

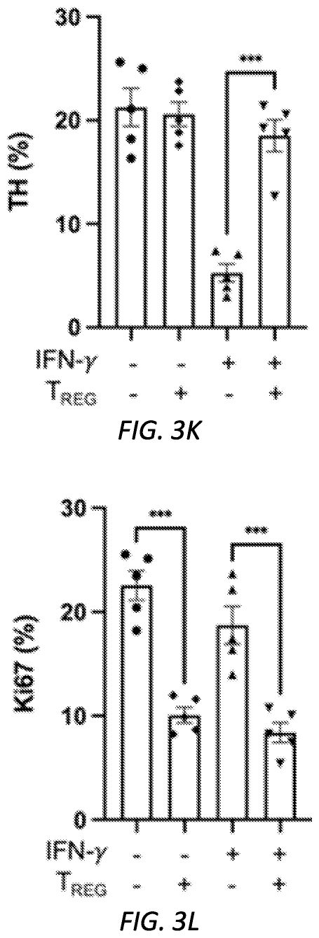

We next examined inflammation-induced cell death in vitro by treating C4-mDAPs with pro-inflammatory cytokines for 2 or 7 days. Following 2 days of treatment with IFN-γ, C4-mDAPs manifested morphological changes with reduced TH expression, as examined by mean fluorescence intensity (MFI) and shortened neurite lengths, but cell death was unaffected (FIGS. 12c-12g). However, after 7 days of treatment with IFN-γ, cleaved caspase-3 was detected, which was accompanied by approximately 12% additional apoptotic cell death, as examined by Annexin-V/7AAD staining (FIGS. 3a,3b). TUNEL assay also revealed about 15% cell death of C4-mDAPs (FIG. 3c). Treatment with TNF-α exhibited similar effects while IL-1β showed weaker effects on C4-mDAPs (FIGS. 12h-12i). Furthermore, the total number of cells as well as TH⁺ cells, TH expression levels, and TH⁺ neurite lengths were all significantly reduced (FIGS. 3d-3h). In contrast, expression of FoxA2 was unaltered (FIGS. 12j-12m). Notably, in agreement with our in vivo data (FIGS. 1b,1c), treatment with pro-inflammatory cytokines for 7 days induced about 15% loss of overall cell number but a much greater loss of TH cells (>80%), suggesting that TH⁺ mDA neurons are more vulnerable than TH cells to pro-inflammatory cytokine-induced cell death. In support of this, when we treated C4-mDAPs with IFN-γ and analyzed them by gating TH′ and TH cells using flow cytometry, most TH″ cells entered apoptotic cell death while TH cells were unaffected (FIG. 3i). We also tested whether co-incubation with autologous T_{REG} can rescue apoptotic cell death triggered by IFN-γ. As shown in FIGS. 3j and 3k, TH⁺ cells were almost completely rescued from cell death in the presence of autologous T_{REG}. Together, our data show that pro-inflammatory cytokines that are acutely induced by needle trauma in vivo can cause preferential loss of TH⁺ mDA neurons in vitro, which can be rescued by T_{REG} CO-incubation. However, intra-striatal co-transplantation with a neutralizing IFN-γ antibody failed to rescue TH⁺ cells (FIG. 12n), indicating that an individual pro-inflammatory cytokine cannot recapitulate the complexity of neuroinflammation in the intact brain.

Interestingly, we also found that co-incubation with autologous T_{REG} significantly decreased the percentage of Ki67³⁰ cells although it did not change that of TH⁺ cells (FIG. 31). Based on previous studies showing the role of TGF-β1 signaling in controlling neural cell proliferation and the size of specific brain areas^{34,35}, we speculated that TGF-β1 secreted by T_{REG} may underlie our observation. In support of this possibility, we found that treatment with anti-TGF-β1 antibody largely restored the percentage of Ki67⁺ cells (FIGS. 12o, 12p).

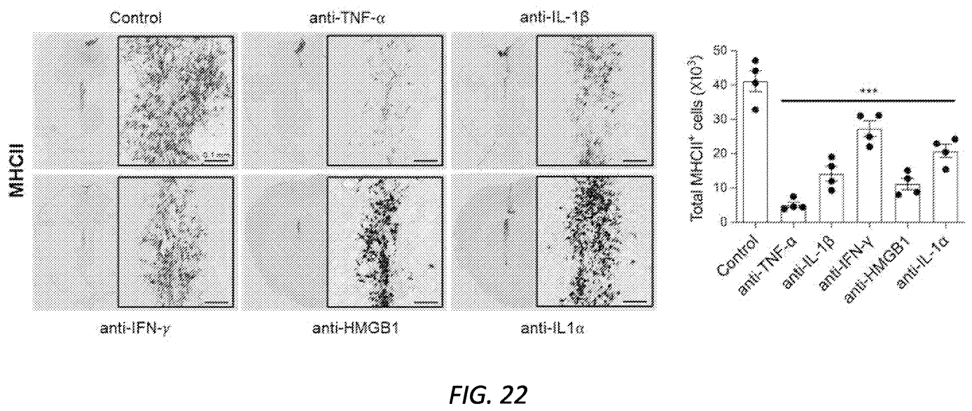

We also investigated the inhibition of needle trauma-induced host inflammatory response using neutralizing monoclonal antibodies. After needle trauma in NSG dKO mice, the degree of inflammation was examined by the degree of infiltration of MHC-II-positive cell as described herein. Specifically, Fischer 344 rats were sacrificed 7 days after intra-striatal co-transplantation of TP medium with or without monoclonal antibodies (anti-TNF-α, -IL-1β, -IFN-g, -HMGB1, -IL1α) from each rat (2 mg/rat). The level of inflammatory cell infiltration into needle track was determined by immunohistochemical staining with anti-MHC-II antibody. (One-way ANOVA, Tukey's post-hoc test; n=4 per group). Each error bar represents means±s.e.m., ***, P<0.001 (FIG. 22). All antibodies tested (anti-TNF-α, anti-IL-1β, anti-IFN-γ, anti-HMGB1, and anti-IL-1 x) significantly suppressed inflammation (FIG. 22). Because our data demonstrated that suppression of inflammation correlates with protection of implanted dopamine neurons, these data strongly suggest that treatment with these antibodies by will lead to better survival of mDANs following transplantation.

### Example 5: Therapeutic Effects of Autologous T_{REG} Co-Transplantation in Xenogeneic PD Model

Based on the above data, we tested whether intra-striatal co-transplantation of host-autologous T_{REG} would protect transplanted mDA neurons from needle trauma-induced cell death during cell therapy (both allogeneic and autologous). Since C4-mDAPs are xenogeneic to rats, we first tested and confirmed that their gene expression is not altered by co-incubation with rat T_{REG} for 72 hours at 1:1 or 5:1 mixing ratios (FIGS. 9d-9f). Next, we co-transplanted C4-mDAPs and autologous rat T_{REG} into the striatum of each 6-OHDA-lesioned Fischer 344 rat with or without CsA treatment and monitored amphetamine-induced rotation behavior monthly post-transplantation (FIG. 4a). As expected, transplantation of C4-mDAPs without CsA failed to improve rotation behavior (FIG. 4b). Interestingly, co-transplantation of C4-mDAPs and autologous T_{REG} without CsA also did not improve rotation behavior. When rats were administered CsA daily, transplantation of C4-mDAPs significantly reduced rotation at 20 weeks post-transplantation, and this effect was further enhanced by co-transplantation with autologous T_{REG}.

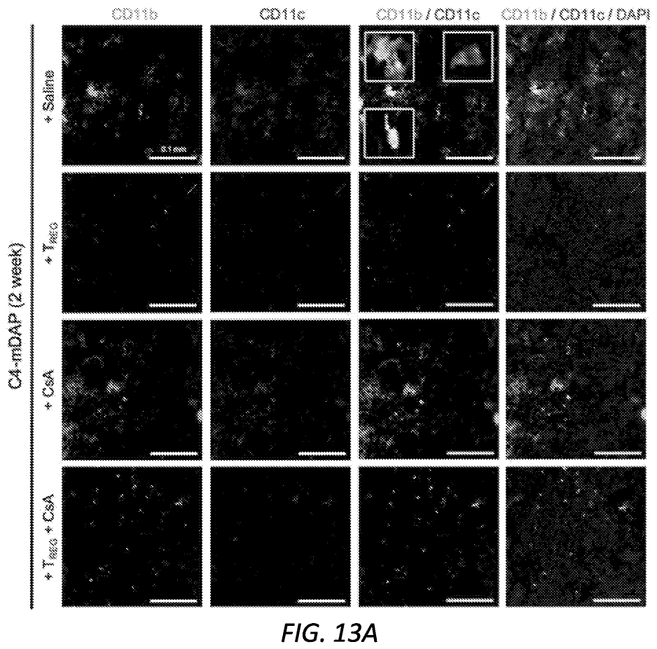

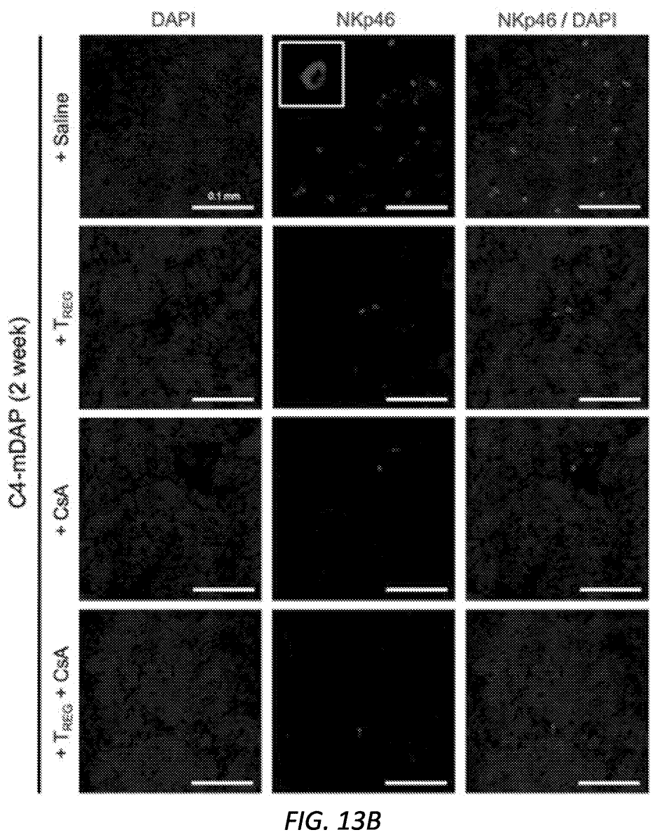

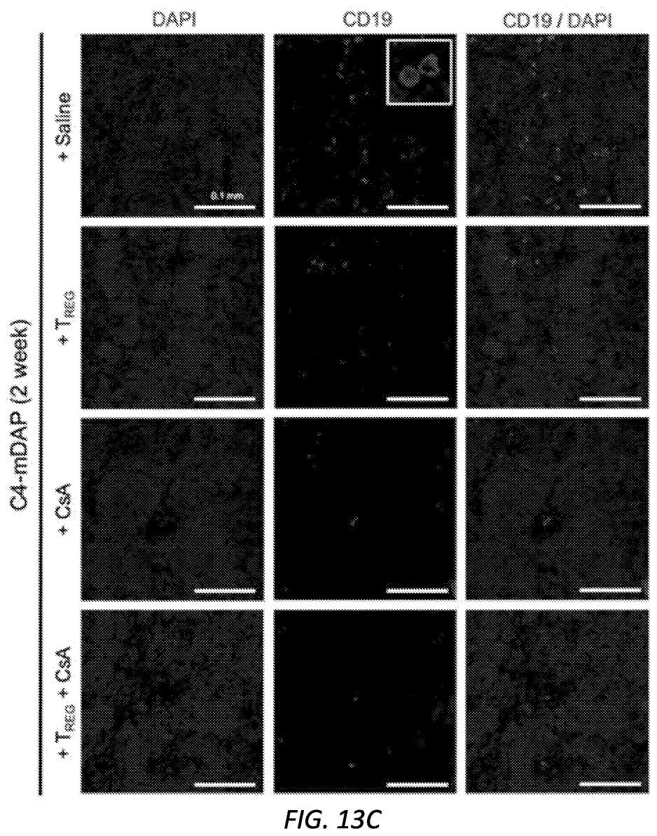

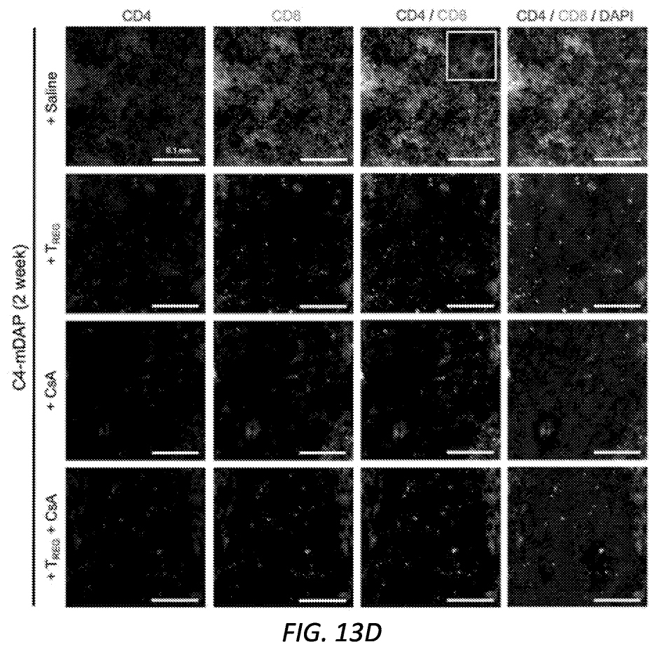

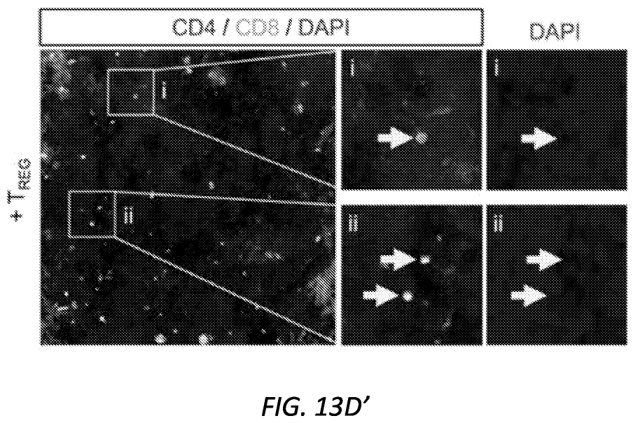

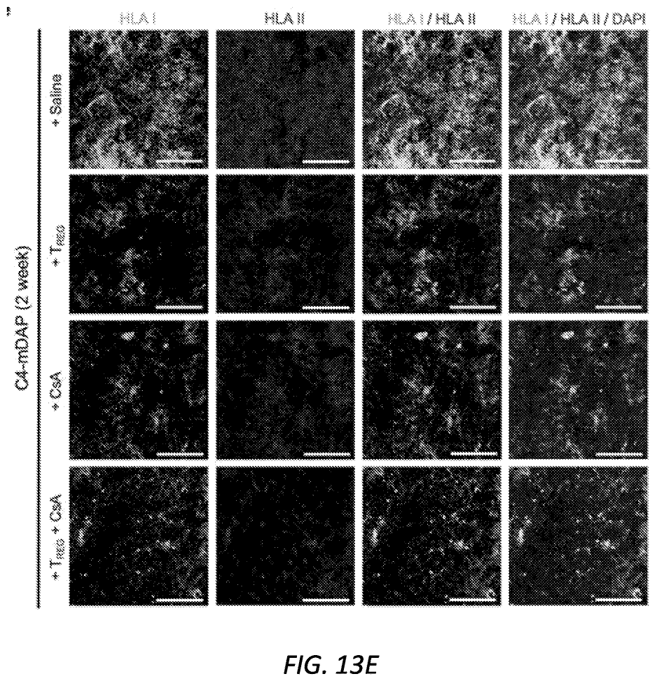

To test the early effects of T_{REG} on grafts, we performed histological analyses at 2 weeks post-transplantation. When transplanting C4-mDAPs only, MHCII and TUNEL staining detected substantial infiltration of inflammatory cells and cell death, respectively, which were significantly rescued by CsA treatment and/or T_{REG} CO-transplantation (FIGS. 4c,4d). Similar patterns were observed for other inflammatory cells including myeloid cells (CD11b⁺/CD11c⁺), NK cells (NKp46⁺), B cells (CD19⁺), and T cells (CD4⁺/CD8⁺) (FIGS. 13a-13d). In addition, expression of immunogenicity-related molecules (HLA I and II) was prominently decreased by CsA treatment and/or T_{REG} CO-transplantation (FIG. 13e). Immunohistochemical analysis of hNUCLEI⁺ cells showed that CsA and/or T_{REG} CO-transplantation resulted in the survival of most of the grafted cells, while transplants composed of C4-mDAPs only (-CsA,-T_{REG}) were rejected (FIG. 4e). Accordingly, TH⁺ cells were almost completely eliminated when transplanting C4-mDAPs only (FIG. 4f). Interestingly, although CsA was injected daily and T_{REG} were injected only once, each treatment prevented graft rejection and spared TH⁺ cells at this 2 week time point, with higher efficiency observed with T_{REG} than CsA treatment (FIGS. 4e,4f).

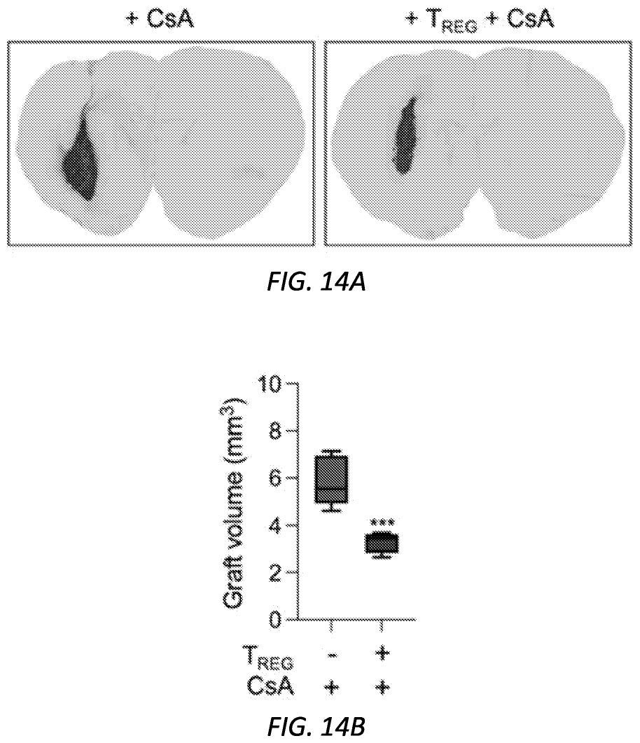

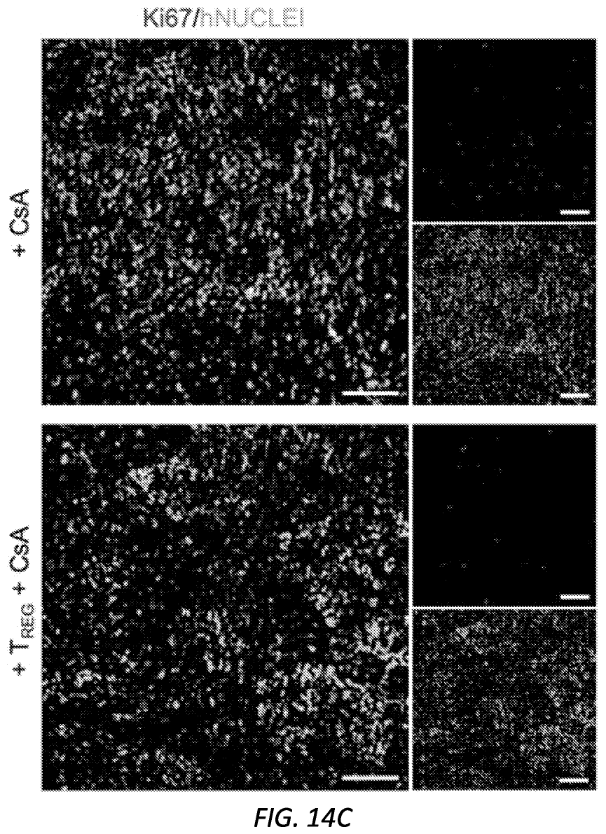

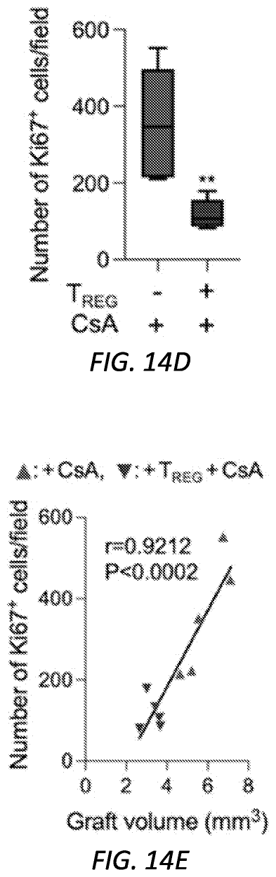

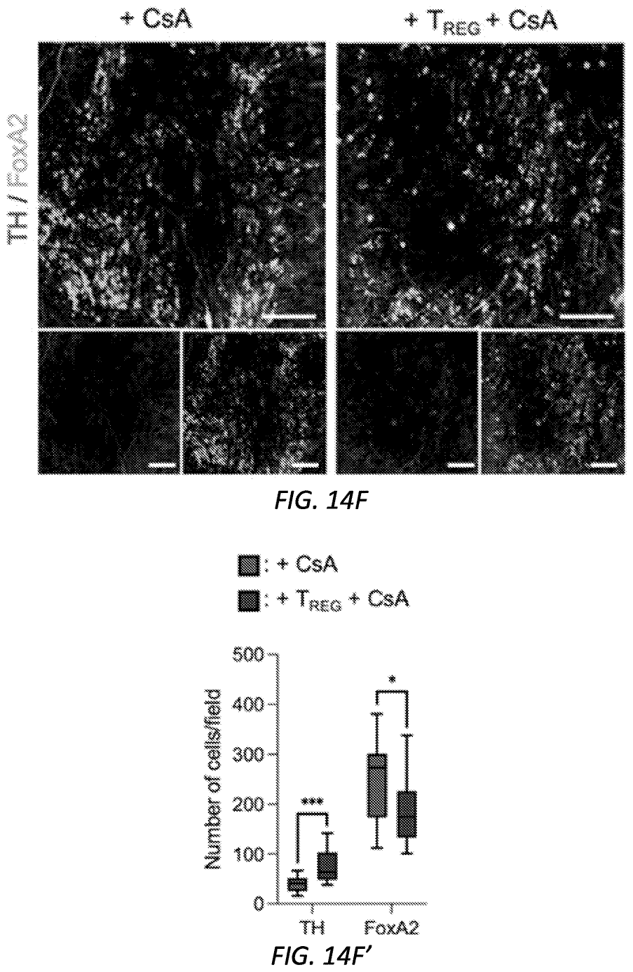

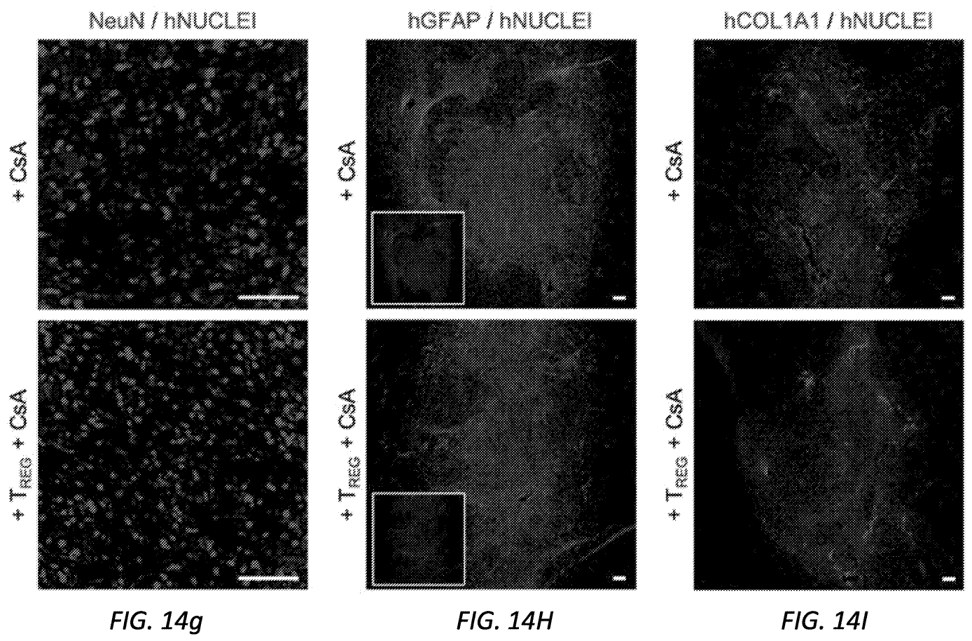

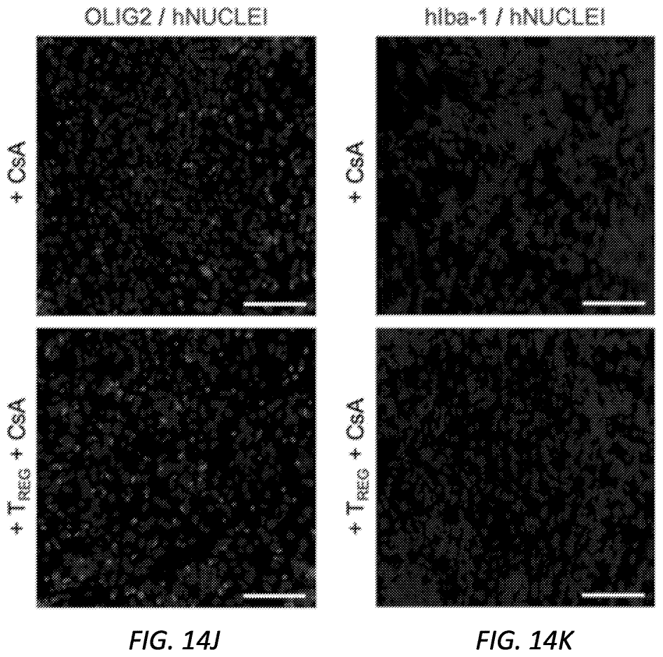

Next, we analyzed grafts (C4-mDAPs into Fischer 344 rats) at 20 weeks post-transplantation (after behavioral studies, shown in FIG. 4a). As expected from the 2 weeks graft analysis, all grafts were rejected when transplanting C4-mDAPs only (-CSA, -T_{REG}) (FIG. 4g). Notably, all grafts were rejected even when T_{REG} were co-transplanted, a difference from the 2 weeks outcome suggesting that T_{REG} alone can provide only short-term graft protection. However, in this xenogeneic model grafts survived with daily CsA administration and approximately 2,500 TH⁺ cells were detected (FIG. 4h). Surprisingly, we found that in the grafts with T_{REG} CO-transplantation, both the graft size and the total number of hNUCLEI⁺ cells were significantly smaller, accompanied with fewer Ki67⁺ cells, than grafts without T_{REG} co-transplantation (FIG. 4g, FIGS. 14a-14d). There was a strong correlation between Ki67⁺ cell number and graft volume (FIG. 14e). However, when we examined TH⁺ cells specifically, the total number of TH⁺ cells increased by almost 2-fold when T_{REG} were co-transplanted, compared to grafts without T_{REG} CO-transplantation (FIG. 4h) whereas FoxA2⁻ cell number was similar or slight decreased (FIG. 14f). In general agreement with recent studies^{36,37}, both grafts with and without T_{REG} CO-transplantation contained neurons, glial cells (astrocytes, oligodendrocyte lineage cells, and microglia), and vascular leptomeningeal cells (VLMC) (FIGS. 14g-14k). Our analysis did not detect noticeable changes in cell type composition by T_{REG} CO-transplantation, except the prominent decrease in Ki67⁺ cell number.

### Example 6: Therapeutic Effects of Autologous T_{REG} Co-Transplantation in Autologous PD Models

We next examined the therapeutic effects of T_{REG} in an autologous setting by co-transplanting patient immune-humanized mice with the same patient's mDAPs and T_{REG} (C4-T_{REG}; FIG. 9g). To this end, we produced a PD model using C4-hu mice by stereotactic injection of 6-OHDA into the substantia nigra, and co-transplanted C4-mDAPs and C4-T_{REG} into the striatum (FIG. 15a). Unfortunately, these mice did not survive more than 2 months post-transplantation (FIG. 15b). This early death of C4-hu mice is likely due to the occurrence of graft-versus-host disease (GVHD), as previous studies showed that humanized mice usually die of GVHD at 30-90 days after injection of human PBMC into NSG mice^{38,39}. Although we could not perform behavioral tests and long-term graft analyses, we investigated the effects of C4-T_{REG} co-transplantation at 2 months post-transplantation. Stereological analyses of the grafts showed that the total number of hNUCLEI⁺ cells as well as graft volumes were smaller in the T_{REG} CO-transplanted group compared to the C4-mDAPs only group (FIGS. 15c,15d), which correlated with the number of Ki67⁺ cells (FIGS. 15e,15f). In contrast, the number of TH′ cells was higher in the T_{REG} CO-transplanted group (FIG. 15g). T_{REG} were no longer detected at this time (FIG. 15h). Thus, although we could not test long-term benefits of autologous T_{REG} CO-transplantation, these humanized mouse data are consistent with the data observed with Fischer 344 rats and support the notion that co-transplanting autologous T_{REG} protects mDA neurons from the acute host inflammatory response to needle trauma and allows their improved survival.

We next investigated the therapeutic effects of co-transplantation of C4-mDAPs and C4-T_{REG} using unmodified NSG mice. First, we generated unilaterally 6-OHDA-lesioned NSG mice which exhibited >6 amphetamine-induced ipsilateral rotations per minute consistently during the whole period of testing (24 weeks). Since NSG mice are relatively immunodeficient, no grafts were rejected despite the absence of immunosuppression. When we transplanted 100,000 C4-mDAPs into the striatum of these mice, rotation behavior was significantly reduced (<6 rotation per min) starting at 20 weeks post-transplantation (FIG. 5a). When C4-T_{REG} were co-transplanted, reduction of rotation started earlier (16 weeks post-transplantation) and showed greater improvement than the C4-mDAP-only group in both amphetamine-induced and apomorphine-induced rotation tests (FIGS. 5b,5c). In addition, all transplanted mice showed significantly improved forelimb function in the cylinder test at 20 weeks post-transplantation (FIG. 5d).

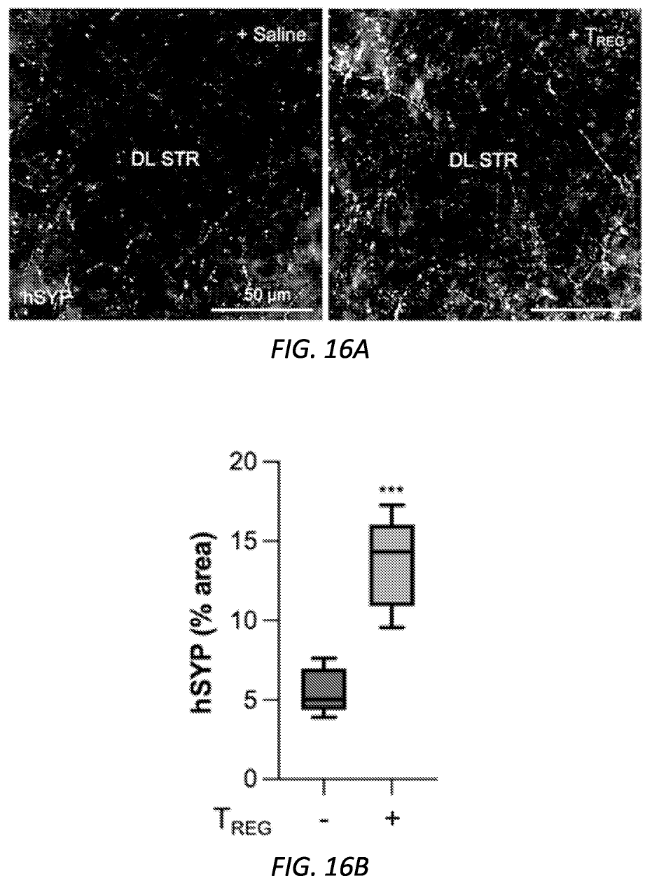

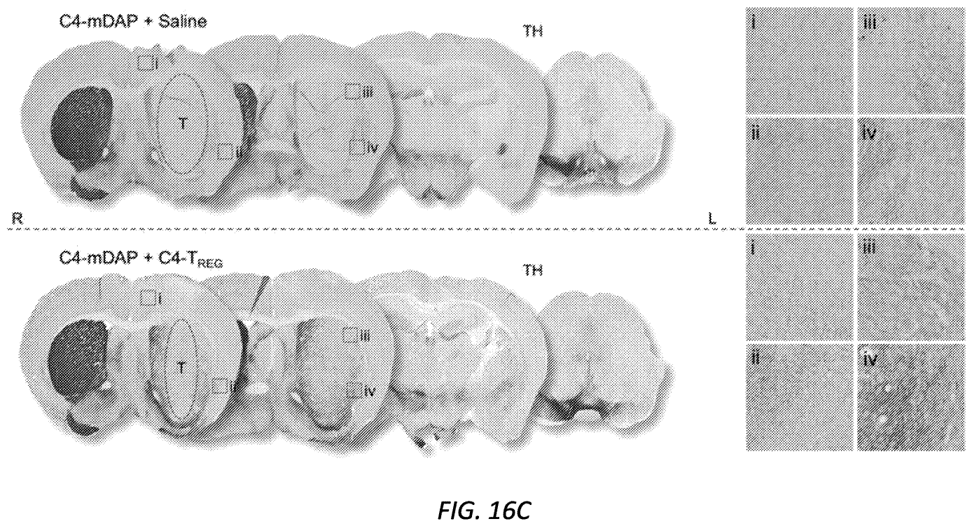

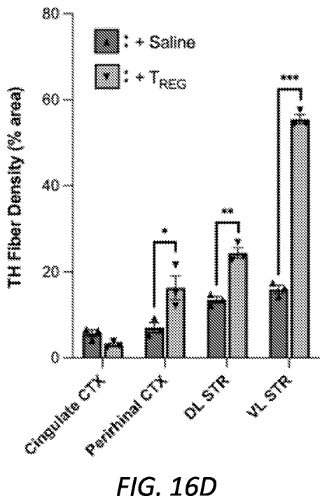

We compared the histology and immunohistochemistry of grafts between the C4-mDAP only and the co-transplantation groups. At 2 weeks, the total number of hNUCLEI⁺ cells was approximately 100,000 without or with C4-T_{REG} CO-transplantation, which increased to approximately 673,000 and 401,000 cells at 20 weeks post-transplantation, respectively (FIG. 5e). Consistent with these data, the graft volume at 20 weeks post-transplantation was 8.53±0.73 and 5.11±0.57 mm³ (FIG. 5f), respectively, in C4-mDAPs only and co-transplantation groups, which correlated with the percentage of Ki67⁺ cells (0.79±0.06%; ±T_{REG}: 0.27±0.04%) (FIGS. 5g,5h). The total number of TH⁺ cells was significantly higher in the grafts of the co-transplantation groups at both 2 weeks (1,825±287; +T_{REG}: 4,427±189) and at 20 weeks post-transplantation (5,421±534; +T_{REG}: 9,523±835) despite smaller overall graft volumes (FIG. 5i). Among TH⁺ neurons, the percentage of GIRK2⁺ ALDHIAl⁻ A9-like and Calbindin⁺ A10-like DA subtypes were unaltered by co-transplantation (FIGS. 5j,5k). The ratio of neuronal cells over total cells (NeuN⁺/hNUCLEI⁺ cells) was significantly higher in grafts of co-transplanted mice (27.39±1.99%; +T_{REG}: 49.93±1.96%, respectively) (FIG. 5l). Furthermore, we found that co-transplanted grafts contained higher densities of graft-derived human synaptophysin (hSYP)⁺ synapses in the dorsolateral striatum (DL STR) (5.54±0.65%; +T_{REG}: 13.64±1.30%; FIGS. 16a, 16b). At the graft-host boundary of T_{REG} CO-transplanted brains, higher levels of TH⁺hSYP⁺ neuronal terminals were identified on host dendritic spines (DARPP32⁺ neurons), their preferential targets, consistent with synaptic connections with the host striatal neurons (FIG. 5m). When we assessed integration of transplanted C4-mDAP cells into host brain using TH innervation density, the TH⁺ fiber density in the striatum was higher in both DL STR (13.50±0.75%; +T_{REG}: 24.38±1.24) and VL STR (15.96±0.94; +T_{REG}: 55.50±1.07%) in the co-grafted group (FIGS. 16c,16d).

### Example 7: Summary of The Experimental Data

In the 1980's and 1990's, numerous investigators studied the survival of transplanted cells as a crucial requirement for successful PD cell therapy, mostly using human and rodent embryonic VM cells⁷⁻¹¹. These early studies revealed that a great majority (80-99%) of transplanted mDA neurons undergo apoptotic cell death within 1-2 weeks post-transplantation. Notably, these studies also revealed that the number of surviving mDA neurons subsequently remained the same at later time points (e.g., at 4 days, 2 and 6 weeks post-transplantation), further supporting the view that most transplanted mDA neurons suffered an early death and that no new mDA neurons were generated from transplanted VM cells. Many potential mechanisms were proposed to explain the poor survival and early death of grafted mDA neurons, mostly focusing on interactions between grafted cells and the host brain such as lack of proper supply with oxygen, glucose, or growth factors from the host brain in the immediate vicinity of the fresh graft. Accordingly, numerous efforts have been made to improve graft survival using diverse protective factors including calcium channel antagonists, lazaroids, caspase inhibitors, and trophic factors⁴⁰⁻⁴⁴ In contrast to these extensive earlier studies with fetal-derived tissue, the survival of mDA cells in grafts derived from hiPSC/hESC has not been systematically studied. While we have previously demonstrated that the use of an autologous iPSC source addresses the problem of cell-mediated rejection without immunosuppression¹⁶, the immediate activation of the innate immune system as part of the inflammatory response to surgical injury is not overcome by this measure.

Here, we show that the penetrating trauma of surgical implantation induces a host inflammatory response including a robust innate immune response similar to that described in other models of TBI^{25,26,45,46}, and that this immediate inflammatory response is specifically harmful to the desired therapeutic TH⁺ cell component of the graft. In line with earlier survival studies using embryonic VM cells, we found that less than 10% of transplanted TH⁺ neurons survived the early stage of transplantation while TH cells mostly survived, which was validated in independent experiments using two hiPSC lines (C4 and B1) and one hESC line (H9). In vitro studies using pro-inflammatory cytokines (e.g., IFN-γ, TNF-α, and IL-1β) showed that this cytokine challenge triggers apoptotic cell death preferentially for TH⁺ neurons, which was significantly rescued by co-incubation with autologous T_{REG}. In contrast to VM cell transplantation, however, the number of both TH⁺ and other cells substantially increases at later time points. We speculated that mDAPs derived from hiPSC/hESC have greater potential for differentiation and proliferation than embryonic VM cells and do generate new TH⁺ neurons from TH mDAP cells in the graft after transplantation. In support of this concept, our previous studies showed that neural precursors derived from mouse ES cells exhibit much greater expansion and differentiation potential than those from mouse embryonic VM cells^{47,48}.

Our data also revealed that intra-striatal co-transplantation with T_{REG} autologous to the host greatly ameliorates the inflammatory response to needle trauma, promotes survival of TH⁺mDA neurons, and reduces death of host brain cells as well. Since the inflammatory and immune cell activation caused by the needle trauma is nonspecific but preferentially harmful to TH⁺neurons compared to TH″ cells in the graft, it is detrimental regardless of whether the grafted cells are autologous, allogeneic, or xenogeneic. Our data showed that T_{REG} autologous to the host effectively mitigate this needle trauma-induced process in both autologous and xenogeneic grafts, leading to better amelioration of motor deficits. Thus, we speculate that co-transplantation of a patient's T_{REG} may improve clinical outcomes in both autologous and allogeneic cell transplantation scenarios, as was recently shown in a rodent model of TBI⁴⁹. However, since autologous T_{REG} CO-transplantation allowed only short-term, but not long-term, survival of xenografts in Fischer 344 rats, systemic immunosuppression will likely still be required for long-term graft survival in allogeneic cell transplantation.

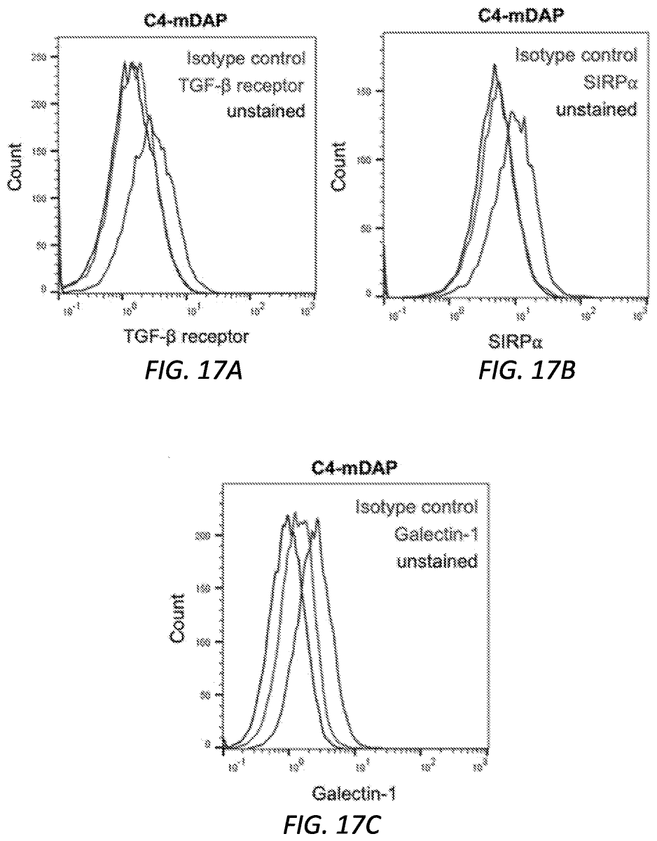

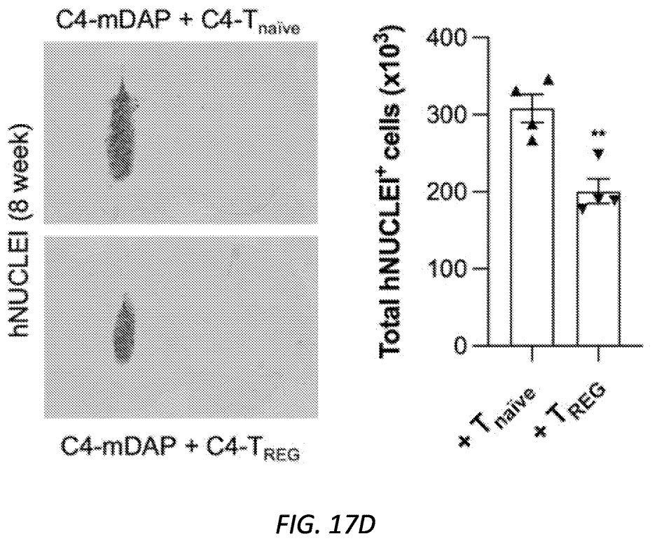

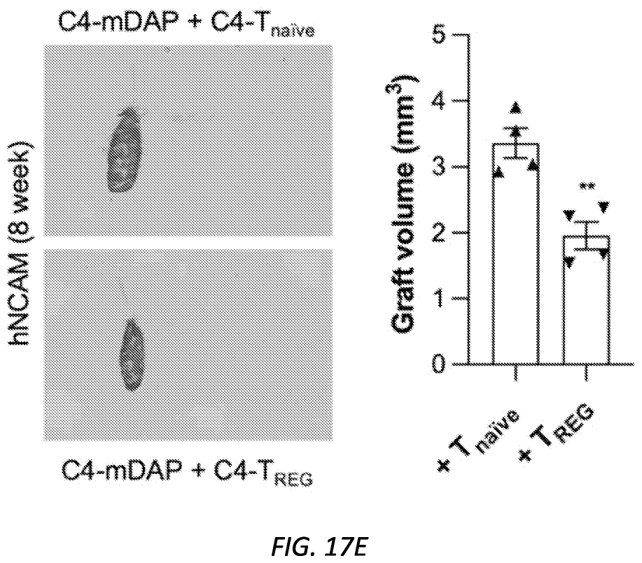

While further studies are warranted, we speculate that T_{REG} exert the neuroprotective effects through multiple direct and/or indirect pathways as follows. First, the immunosuppressive cytokines (e.g., TGF-β1) secreted by T_{REG} directly affect mDAPs. In support of this, previous studies showed that mature mDA neurons express TGF-β receptor⁵⁰ and that TGF-β1 protects mDA neurons from pro-inflammatory cytokine (e.g., IFN-γ)-driven neurotoxicity⁵¹. We confirmed that C4-mDAPs also express TGF-β receptor (FIG. 17a). Second, T_{REG} may indirectly regulate neuroinflammation by suppressing the expression of pro-inflammatory cytokines by surrounding resident brain cells (e.g., astrocytes and microglia) in response to the needle trauma as shown in our data (FIG. 1j, FIGS. 7e,7f). Third, these effects may help prevent infiltration of inflammatory cells from the periphery (FIGS. 1h-1n, FIG. 10b). In addition, recent studies showed that cell-to-cell interactions (e.g., CD47-SIRPα and CD45-galectin1) may directly protect mDA neurons from MPTP neurotoxicity^{52,53}. We found that C4-mDAPs express both SIRPα and Galectin-1 (FIGS. 17b, 17c), suggesting that T_{REG} expressing CD47 and CD45 may directly protect mDA cells through these cell-to-cell contact mechanisms.

An unanticipated finding in this study was that T_{REG} CO-transplantation substantially reduced the expansion of the grafts related to TH⁻ cells, resulting in significantly smaller graft volume with a higher proportionate TH⁺ cell content with improved outgrowth from the grafts. This raises the issue of how T_{REG} co-transplantation affects the cell fate decision of transplanted progenitor cells in the graft. To determine whether the T_{REG} effect on graft proliferation might be due to a dilution effect, we compared co-transplantation of C4-T_{REG} and C4-naïve T cells (C4-T_{naïve}), which are very similar in size but functionally different. When the same numbers of C4-T_{REG} or C4-T_{naïve} were co-transplanted with C4-mDAPs, the cell number and size of grafts were smaller only when C4-T_{REG} were co-transplanted (FIGS. 17d,17e), strongly suggesting that it is not due to a dilution effect. Since C4-mDAPs express the TGF-β receptor (FIG. 17a), T_{REG} may control graft cell proliferation via secretion of TGF-β which is known to control neural cell proliferation^{34,35}. In support of this, treatment with anti-TGF-β1 antibody largely restored the percentage of Ki67⁺ cells (FIGS. 12o,12p). As these T_{REG} effects are not related to the source (autologous vs allogeneic) of the mDA cells per se, they pertain to allogeneic grafting scenarios as well. In contrast to other strategies for immune suppression that involve systemic administration, T_{REG} CO-transplantation is a site-specific strategy less likely to provoke deleterious systemic side effects. Furthermore, our demonstration that in the acute-phase (2-week results) a single administration of T_{REG} had benefits like daily CsA, and that the T_{REG} themselves did not persist in the long-term grafts, suggest that the benefits are related to acute-phase modulation of the innate immune response and that subsequent administration of these cells is not required to sustain the effects. The strategy thus reduces the time and extent at risk of adverse effects.

In sum, our study, inter alia, suggests that initial, specific mDA neuron survival in grafts is strongly affected by the host innate immune response, whereas long-term graft survival is subject to the adaptive immune response, revealing a two-phase involvement of the immune system in graft survival. Co-transplantation of mDAPs with T_{REG} modulates the host immune response triggered by needle trauma and significantly protects mDANs while suppressing proliferation of TH cells, which would benefit both efficacy and safety of cell replacement therapy for PD and other neurodegenerative disorders.

### REFERENCES (All Sections Except the Detailed Description)

1. Parmar, M., Grealish, S. & Henchcliffe, C. The future of stem cell therapies for Parkinson disease. Nat. Rev. Neurosci. 21, 103-115 (2020).
2. Sonntag, K. C. et al. Pluripotent stem cell-based therapy for Parkinson's disease: Current status and future prospects. Prog. Neurobiol. 168, 1-20 (2018).
3 Barker, R.A., Drouin-Ouellet, J. & Parmar, M. Cell-based therapies for Parkinson disease-past insights and future potential. Nat. Rev. Neurol. 11, 492-503 (2015).
4. Lindvall, O. Clinical translation of stem cell transplantation in Parkinson's disease. J. Intern. Med. 279, 30-40 (2016).
5. Kim, T.W., Koo, S.Y. & Studer, L. Pluripotent Stem Cell Therapies for Parkinson Disease: Present Challenges and Future Opportunities. Front. Cell Dev. Biol. 8, 729 (2020).
6. Bjorklund, A. & Parmar, M. Dopamine Cell Therapy: From Cell Replacement to Circuitry Repair. J. Parkinsons Dis. 11, S159-S165 (2021).
7. Brundin, P. et al. Improving the survival of grafted dopaminergic neurons: a review over current approaches. Cell Transplant. 9, 179-195 (2000).
8. Wenker, S.D. & Pitossi, F.J. Cell therapy for Parkinson's disease is coming of age: current challenges and future prospects with a focus on immunomodulation. Gene Ther. 27, 6-14 (2020).
9. Duan, W. M., Widner, H. & Brundin, P. Temporal pattern of host responses against intrastriatal grafts of syngeneic, allogeneic or xenogeneic embryonic neuronal tissue in rats. Exp. Brain Res. 104, 227-242 (1995).
10. Barker, R. A., Dunnett, S. B., Faissner, A. & Fawcett, J. W. The time course of loss of dopaminergic neurons and the gliotic reaction surrounding grafts of embryonic mesencephalon to the striatum. Exp. Neurol. 141, 79-93 (1996).
11. Emgard, M., Karlsson, J., Hansson, O. & Brundin, P. Patterns of cell death and dopaminergic neuron survival in intrastriatal nigral grafts. Exp. Neurol. 160, 279-288 (1999).
12. Tao, Y. et al. Autologous transplant therapy alleviates motor and depressive behaviors in parkinsonian monkeys. Nat. Med. (2021).
13. Hallett, P. J. et al. Successful function of autologous iPSC-derived dopamine neurons following transplantation in a non-human primate model of Parkinson's disease. Cell Stem Cell 16, 269-274 (2015).
14. Emborg, M. E. et al. Induced pluripotent stem cell-derived neural cells survive and mature in the nonhuman primate brain. Cell Rep. 3, 646-650 (2013).
15. Morizane, A. et al. MHC matching improves engraftment of iPSC-derived neurons in non-human primates. Nat. Commun. 8, 385 (2017).
16. Schweitzer, J. S. et al. Personalized iPSC-Derived Dopamine Progenitor Cells for Parkinson's Disease. N. Engl. J. Med. 382, 1926-1932 (2020).
17. Hiller, B. M. et al. Optimizing maturity and dose of iPSC-derived dopamine progenitor cell therapy for Parkinson's disease. NPJ Regen. Med. 7, 24 (2022).
18. Piao, J. et al. Preclinical Efficacy and Safety of a Human Embryonic Stem Cell-Derived Midbrain Dopamine Progenitor Product, MSK-DA01. Cell Stem Cell 28, 217-229 e217 (2021).
19. Josefowicz, S. Z., Lu, L. F. & Rudensky, A. Y. Regulatory T cells: mechanisms of differentiation and function. Annu. Rev. Immunol. 30, 531-564 (2012).
20. Wood, K. J. & Sakaguchi, S. Regulatory T cells in transplantation tolerance. Nat. Rev. Immunol. 3, 199-210 (2003).
21. Song, B. et al. Human autologous iPSC-derived dopaminergic progenitors restore motor function in Parkinson's disease models. J. Clin. Invest. 130, 904-920 (2020).
22. Kim, J. et al. Spotting-based differentiation of functional dopaminergic progenitors from human pluripotent stem cells. Nat. Protoc. (2022).
23. Kriks, S. et al. Dopamine neurons derived from human ES cells efficiently engraft in animal models of Parkinson's disease. Nature 480, 547-551 (2011).
24. Doi, D. et al. Pre-clinical study of induced pluripotent stem cell-derived dopaminergic progenitor cells for Parkinson's disease. Nat. Commun. 11, 3369 (2020).
25. Alam, A. et al. Cellular infiltration in traumatic brain injury. J. Neuroinflammation 17, 328 (2020).
26. Karve, I. P., Taylor, J. M. & Crack, P. J. The contribution of astrocytes and microglia to traumatic brain injury. Br. J. Pharmacol. 173, 692-702 (2016).
27. Duggleby, R., Danby, R. D., Madrigal, J. A. & Saudemont, A. Clinical Grade
Regulatory CD4(+) T Cells (Tregs): Moving Toward Cellular-Based Immunomodulatory Therapies. Front. Immunol. 9, 252 (2018).
28. Romano, M., Fanelli, G., Albany, C. J., Giganti, G. & Lombardi, G. Past, Present, and Future of Regulatory T Cell Therapy in Transplantation and Autoimmunity. Front. Immunol. 10, 43 (2019).
29. Itakura, G. et al. Low immunogenicity of mouse induced pluripotent stem cell-derived neural stem/progenitor cells. Sci. Rep. 7, 12996 (2017).
30. Ozaki, M. et al. Evaluation of the immunogenicity of human iPS cell-derived neural stem/progenitor cells in vitro. Stem Cell Res. 19, 128-138 (2017).
31. Yamasaki, S. et al. Low Immunogenicity and Immunosuppressive Properties of Human ESC- and iPSC-Derived Retinas. Stem Cell Reports 16, 851-867 (2021).
32. Zhao, T., Zhang, Z. N., Rong, Z. & Xu, Y. Immunogenicity of induced pluripotent stem cells. Nature 474, 212-215 (2011).
33. Zhao, T. et al. Humanized Mice Reveal Differential Immunogenicity of Cells Derived from Autologous Induced Pluripotent Stem Cells. Cell Stem Cell 17, 353-359 (2015).
34. Falk, S. et al. Brain area-specific effect of TGF-beta signaling on Wnt-dependent neural stem cell expansion. Cell Stem Cell 2, 472-483 (2008).
35. Izsak, J. et al. TGF-betal Suppresses Proliferation and Induces Differentiation in Human iPSC Neural in vitro Models. Front. Cell Dev. Biol. 8, 571332 (2020).
36. Tiklova, K. et al. Single cell transcriptomics identifies stem cell-derived graft composition in a model of Parkinson's disease. Nat. Commun. 11, 2434 (2020).
37. Xu, P. et al. Human midbrain dopaminergic neuronal differentiation markers predict cell therapy outcomes in a Parkinson's disease model. J. Clin. Invest. 132 (2022).
38. Hess, N. J., Brown, M. E. & Capitini, C. M. GVHD Pathogenesis, Prevention and Treatment: Lessons From Humanized Mouse Transplant Models. Front. Immunol. 12, 723544 (2021).
39. Becker, C. et al. Protection from graft-versus-host disease by HIV-1 envelope protein gp120-mediated activation of human CD4+CD25+ regulatory T cells. Blood 114, 1263-1269 (2009).
40. Finger, S. & Dunnett, S. B. Nimodipine enhances growth and vascularization of neural grafts. Exp. Neurol. 104, 1-9 (1989).
41. Nakao, N., Frodl, E. M., Duan, W. M., Widner, H. & Brundin, P. Lazaroids improve the survival of grafted rat embryonic dopamine neurons. Proc. Natl Acad. Sci. USA 91, 12408-12412 (1994).
42. Schierle, G. S. et al. Caspase inhibition reduces apoptosis and increases survival of nigral transplants. Nat. Med. 5, 97-100 (1999).
43. Sinclair, S. R. et al. GDNF enhances dopaminergic cell survival and fibre outgrowth in embryonic nigral grafts. Neuroreport 7, 2547-2552 (1996).
44. Takayama, H. et al. Basic fibroblast growth factor increases dopaminergic graft survival and function in a rat model of Parkinson's disease. Nat. Med. 1, 53-58 (1995).
45. McKee, C. A. & Lukens, J. R. Emerging Roles for the Immune System in Traumatic Brain Injury. Front. Immunol. 7, 556 (2016).
46. Bai, R. et al. Long-Term Kinetics of Immunologic Components and Neurological Deficits in Rats Following Repetitive Mild Traumatic Brain Injury. Med. Sci. Monit. 23, 1707-1718 (2017).
47. Chung, S. et al. Neural precursors derived from embryonic stem cells, but not those from fetal ventral mesencephalon, maintain the potential to differentiate into dopaminergic neurons after expansion in vitro. Stem Cells 24, 1583-1593 (2006).
48. Chung, S. et al. ES cell-derived renewable and functional midbrain dopaminergic progenitors. Proc. Natl Acad. Sci. USA 108, 9703-9708 (2011).
49. Caplan, H. W. et al. Combination therapy with Treg and mesenchymal stromal cells enhances potency and attenuation of inflammation after traumatic brain injury compared to monotherapy. Stem Cells 39, 358-370 (2021).
50. Krieglstein, K., Suter-Crazzolara, C., Fischer, W. H. & Unsicker, K. TGF-beta superfamily members promote survival of midbrain dopaminergic neurons and protect them against MPP+toxicity. EMBO J. 14, 736-742 (1995).
51. Zhou, X., Zoller, T., Krieglstein, K. & Spittau, B. TGFbetal inhibits IFNgamma-mediated microglia activation and protects mDA neurons from IFNgamma-driven neurotoxicity. J. Neurochem. 134, 125-134 (2015).
52. Huang, Y., Liu, Z., Cao, B. B., Qiu, Y. H. & Peng, Y. P. Treg Cells Protect Dopaminergic Neurons against MPP+Neurotoxicity via CD47-SIRPA Interaction. Cell. Physiol. Biochem. 41, 1240-1254 (2017).
53. Huang, Y., Liu, Z., Cao, B. B., Qiu, Y. H. & Peng, Y. P. Treg Cells Attenuate Neuroinflammation and Protect Neurons in a Mouse Model of Parkinson's Disease. J. Neuroimmune Pharmacol. 15, 224-237 (2020).
54. Liu, D. et al. Impact of schizophrenia GWAS loci converge onto distinct pathways in cortical interneurons vs glutamatergic neurons during development. Mol. Psychiatry (2022).

### Other Embodiments

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having Parkinson's Disease (PD), the method comprising administering a population of regulatory T (T_{REG}) cells and a population of midbrain dopamine (mDA) cells to the brain of the subject.

2. The method of claim 1, wherein the population of mDA cells comprises midbrain dopamine neurons (mDANs) and midbrain dopamine progenitor cells (mDAPs).

3. The method of claim 1 or claim 2, wherein the T_{REG} cells and the mDA cells are administered to the subject at the same time, optionally mixed together in a single composition.

4. The method of claim 1 or claim 2, wherein the T_{REG} cells are first administered to the subject and subsequently the mDA cells are administered to the subject, or the mDA cells are first administered to the subject and subsequently the T_{REG} cells are administered to the subject.

5. The method of any one of the preceding claims, wherein the T_{REG} cells and the mDA cells are autologous T_{REG} cells and autologous mDA cells.

6. The method of claim 5, wherein the autologous mDA are obtained by in vitro differentiation of human induced pluripotent stem cells (hiPSCs) into mDA cells, wherein the hiPSC are derived from cells from the subject.

7. The method of any one of claims 1-5, wherein the T_{REG} cells and the mDA cells are allogeneic or T_{REG} cells and allogeneic mDA cells, optionally wherein the autologous mDA are obtained by in vitro differentiation of human pluripotent stem cells (hPSCs), optionally human embryonic stem cells (hESCs) and induced pluripotent stem cells (hiPSCs), into mDA cells.

8. The method of any one of claims 1-5, wherein the T_{REG} cells and the mDA cells are xenogeneic T_{REG} cells and xenogeneic mDA cells.

9. The method of any one of claims 1-8, wherein the T_{REG} cells and mDA cells are administered intrastriatally, optionally by intra-striatal injection.

10. A method of treating a subject having Parkinson's Disease (PD), the method comprising administering to the subject a population of midbrain dopamine (mDA) cells and an antibody (Ab) (optionally, an antibody that binds a pro-inflammatory cytokine or a damage-associated molecular patterns (DAMPs) molecule; optionally an anti-TNF-α Ab (optionally Infliximab), an anti-IL-1β Ab (optionally Canakinumab), an anti-IFN-γ Ab (optionally Emapalumab), an anti-HMGB1 Ab, or an anti-IL-1α Ab).

11. A method of decreasing an immune response in a subject (e.g., an immune response triggered by needle trauma, an immune response at the site of administration) during cell transplantation, the method comprising administering to the subject an effective amount of a population of T_{REG} cells with a second population of cells.

12. The method of claim 11, wherein the second population of cells is a population of mDA cells.

13. The method of claim 12, wherein the T_{REG} cells and the mDA cells are transplanted simultaneously.

14. The method of claim 12, wherein the T_{REG} cells and the mDA cells are transplanted separately,
wherein the T_{REG} cells are first administered to the subject and subsequently the mDA cells are administered to the subject, or
wherein the mDA cells are first administered to the subject and subsequently the T_{REG} cells administered to the subject.

15. The method of any one of claims 11-14, wherein the T_{REG} cells are autologous T_{REG} cells, allogenic T_{REG} cells, or xenogeneic T_{REG} cells.

16. The method of any one of claims 12-15, wherein the mDA cells are autologous mDA cells, allogenic mDA cells, or xenogeneic mDA cells.

17. The method of any one of claims 11-16, wherein the T_{REG} cells are transplanted intrastriatally.

18. The method of any one of claims 12-17, wherein the mDA cells are transplanted intrastriatally.

19. A method of decreasing an immune response in a subject triggered by needle trauma during a cell transplantation comprising administering to the subject an effective amount of an antibody (Ab) (optionally an antibody that binds a pro-inflammatory cytokine or a damage-associated molecular patterns (DAMPs) molecule; optionally an anti-TNF-α Ab (optionally Infliximab), an anti-IL-1β Ab (optionally Canakinumab), an anti-IFN-γ Ab (optionally Emapalumab), an anti-HMGB1 Ab, or an anti-IL-1α Ab) with a population of cells (optionally mDA cells).

20. A method of increasing cell survival in cell transplantation or increasing outgrowths of transplanted cells or reducing overgrowth during a cell transplantation, the method comprising co-transplanting an effective amount of a population of T_{REG} cells with a second population of cells (“transplanted cells”; optionally mDA cells).

21. The method of claim 20, wherein the transplanted cells are mDA cells.

22. The method of claim 20 or claim 21, wherein co-transplanting results in a graft, and wherein there is an increase in TH⁺ cells in the graft compared to a graft without the co-transplanting;
wherein co-transplanting results in a graft, and wherein there is an increase in the TH⁻ cells: TH⁻ cells ratio in the graft and/or an increase in the TH⁺ cells: Ki67⁻ cells ratio in the graft compared to a graft without the co-transplanting; and/or
wherein co-transplanting results in a graft, and wherein there is a decrease in Ki67⁺ cells in the graft and/or a decrease in TH cells in the graft compared to a graft without the co-transplanting.

23. The method of any one of claims 20-22, wherein the T_{REG} cells and the transplanted cells (optionally mDA cells) are transplanted simultaneously.

24. The method of any one of claims 20-22, wherein the T_{REG} cells and the transplanted cells (optionally mDA cells) are transplanted separately,
wherein the T_{REG} cells are first administered to the subject and subsequently the transplanted cells (optionally mDA cells) are administered to the subject, or
wherein the transplanted cells (optionally mDA cells) are first administered to the subject and subsequently the T_{REG} cells administered to the subject.

25. The method of any one of claims 20-24, wherein the T_{REG} cells are autologous T_{REG} cells, allogenic T_{REG} cells, or xenogeneic T_{REG} cells.

26. The method of any one of claims 20-25, wherein the transplanted cells (optionally mDA cells) are autologous transplanted cells, allogenic transplanted cells (optionally mDA cells), or xenogeneic transplanted cells (optionally mDA cells).

27. The method of any one of claims 20-26, wherein the T_{REG} cells are transplanted intrastriatally.

28. The method of any one of claims 20-27, wherein the transplanted cells (optionally mDA cells) are transplanted intrastriatally.

29. A method of increasing cell survival in cell transplantation or increasing outgrowths of transplanted cells or reducing overgrowth during a cell transplantation, the method comprising administering to the subject an effective amount of an antibody (Ab) (optionally, an antibody that binds a pro-inflammatory cytokine or a damage-associated molecular patterns (DAMPs) molecule; optionally, an anti-TNF-α Ab (optionally Infliximab), an anti-IL-1β Ab (optionally Canakinumab), an anti-IFN-γ Ab (optionally Emapalumab), an anti-HMGB1 Ab, or an anti-IL-1α Ab) and a population of cells (optionally mDA cells).


SEQUENCE LISTING (8 sequences)

SEQ ID NO: 1 - DNA, 22 bp, synthetic construct
  source 1..22: mol_type = other DNA
cgggcttctcggaccaggtgta

SEQ ID NO: 2 - DNA, 22 bp, synthetic construct
  source 1..22: mol_type = other DNA
ctcctcggcggtgtactccaca

SEQ ID NO: 3 - DNA, 20 bp, synthetic construct
  source 1..20: mol_type = other DNA
ggtgctttggctgacttttt

SEQ ID NO: 4 - DNA, 20 bp, synthetic construct
  source 1..20: mol_type = other DNA
gttgctcacggaggagtagc

SEQ ID NO: 5 - DNA, 21 bp, synthetic construct
  source 1..21: mol_type = other DNA
agggtgacgagtttgtcctga

SEQ ID NO: 6 - DNA, 21 bp, synthetic construct
  source 1..21: mol_type = other DNA
catcgcttttgcctgagtctg

SEQ ID NO: 7 - DNA, 21 bp, synthetic construct
  source 1..21: mol_type = other DNA
catgtacgttgctatccaggc

SEQ ID NO: 8 - DNA, 21 bp, synthetic construct
  source 1..21: mol_type = other DNA
ctccttaatgtcacgcacgat